SEQUENCE LISTING (131 sequences)

SEQ ID NO: 1 - protein, 575 aa, synthetic construct
MNYFPDEVIEHVFDFVTSHKDRNAISLVCKSWYKIERYSRQKVFIGNCYAINPERLLRRFPCLKSLTLKGKPHFADFNLVPHEWGGFVLPWIEALARSRVGLEELRLKRMVVTDESLELLSRSFVNFKSLVLVSCEGFTTDGLASIAANCRHLRDLDLQENEIDDHRGQWLSCFPDTCTTLVTLNFACLEGETNLVALERLVARSPNLKSLKLNRAVPLDALARLMACAPQIVDLGVGSYENDPDSESYLKLMAVIKKCTSLRSLSGFLEAAPHCLSAFHPICHNLTSLNLSYAAEIHGSHLIKLIQHCKKLQRLWILDSIGDKGLEVVASTCKELQELRVFPSDLLGGGNTAVTEEGLVAISAGCPKLHSILYFCQQMTNAALVTVAKNCPNFIRFRLCILEPNKPDHVTSQPLDEGFGAIVKACKSLRRLSLSGLLTDQVFLYIGMYANQLEMLSIAFAGDTDKGMLYVLNGCKKMKKLEIRDSPFGDTALLADVSKYETMRSLWMSSCEVTLSGCKRLAEKAPWLNVEIINENDNNRMEENGHEGRQKVDKLYLYRTVVGTRMDAPPFVWIL

SEQ ID NO: 2 - protein, 1086 aa, synthetic construct
MKAPSNGFLPSSNEGEKKPINSQLWHACAGPLVSLPPVGSLVVYFPQGHSEQVAASMQKQTDFIPNYPNLPSKLICLLHSVTLHADTETDEVYAQMTLQPVNKYDREALLASDMGLKLNRQPTEFFCKTLTASDTSTHGGFSVPRRAAEKIFPPLDFSMQPPAQEIVAKDLHDTTWTFRHIYRGQPKRHLLTTGWSVFVSTKRLFAGDSVLFVRDEKSQLMLGIRRANRQTPTLSSSVISSDSMHIGILAAAAHANANSSPFTIFFNPRASPSEFVVPLAKYNKALYAQVSLGMRFRMMFETEDCGVRRYMGTVTGISDLDPVRWKGSQWRNLQVGWDESTAGDRPSRVSIWEIEPVITPFYICPPPFFRPKYPRQPGMPDDELDMENAFKRAMPWMGEDFGMKDAQSSMFPGLSLVQWMSMQQNNPLSGSATPQLPSALSSFNLPNNFASNDPSKLLNFQSPNLSSANSQFNKPNTVNHISQQMQAQPAMVKSQQQQQQQQQQHQHQQQQLQQQQQLQMSQQQVQQQGIYNNGTIAVANQVSCQSPNQPTGFSQSQLQQQSMLPTGAKMTHQNINSMGNKGLSQMTSFAQEMQFQQQLEMHNSSQLLRNQQEQSSLHSLQQNLSQNPQQLQMQQQSSKPSPSQQLQLQLLQKLQQQQQQQSIPPVSSSLQPQLSALQQTQSHQLQQLLSSQNQQPLAHGNNSFPASTFMQPPQIQVSPQQQGQMSNKNLVAAGRSHSGHTDGEAPSCSTSPSANNTGHDNVSPTNFLSRNQQQGQAASVSASDSVFERASNPVQELYTKTESRISQGMMNMKSAGEHFRFKSAVTDQIDVSTAGTTYCPDVVGPVQQQQTFPLPSFGFDGDCQSHHPRNNLAFPGNLEAVTSDPLYSQKDFQNLVPNYGNTPRDIETELSSAAISSQSFGIPSIPFKPGCSNEVGGINDSGIMNGGGLWPNQTQRMRTYTKVQKRGSVGRSIDVTRYSGYDELRHDLARMFGIEGQLEDPLTSDWKLVYTDHENDILLVGDDPWEEFVNCVQNIKILSSVEVQQMSLDGDLAAIPTTNQACSETDSGNAWKVHYEDTSAAASFNR

SEQ ID NO: 3 - protein, 97 aa, synthetic construct
IYVKVSMDGAPYLRKIDLSCYKGYSELLKALEVMFKFSVGEYFERDGYKGSDFVPTYEDKDGDWMLIGDVPWEMFICTCKRLRIMKGSEAKGLGCGV

SEQ ID NO: 4 - protein, 15 aa, synthetic construct
EAAAKEAAAKEAAAK

SEQ ID NO: 5 - protein, 20 aa, synthetic construct
GRGPGGGHQTSLYKKAGFKM

SEQ ID NO: 6 - protein, 19 aa, synthetic construct
GRGPGGGHQYPYDVPDYAM

SEQ ID NO: 7 - protein, 28 aa, synthetic construct
GRGPGGGHQYPYDVPDYAYPYDVPDYAM

SEQ ID NO: 8 - protein, 13 aa, Arabidopsis thaliana
RELVFLILQFLDE

SEQ ID NO: 9 - protein, 13 aa, Arabidopsis thaliana
RDVIKIMLQFCKE

SEQ ID NO: 10 - protein, 13 aa, Schistosoma mansoni
QEINRLILEYLVV

SEQ ID NO: 11 - protein, 13 aa, Homo sapiens
SDVIRLIMQYLKE

SEQ ID NO: 12 - protein, 13 aa, Homo sapiens
KELLLLIRNHLIS

SEQ ID NO: 13 - protein, 13 aa, Arabidopsis thaliana
KELLLLIHEHLQA

SEQ ID NO: 14 - protein, 13 aa, Anopheles merus
QQLYQLIYQHLIA

SEQ ID NO: 15 - protein, 13 aa, Lingula unguis
KELLQLIHDHLVS

SEQ ID NO: 16 - protein, 13 aa, Mus musculus
KNLLILILHYLTQ

SEQ ID NO: 17 - protein, 13 aa, Schizosaccharomyces pombe
NQVNYIIWRYLKE

SEQ ID NO: 18 - protein, 13 aa, Stentor coeruleus
NDINLLIYHYLKE

SEQ ID NO: 19 - protein, 13 aa, Dictyostelium discoideum
DEINFLIYKYLLE

SEQ ID NO: 20 - protein, 13 aa, Saccharomyces cerevisiae
EELNYLIWRYCQE

SEQ ID NO: 21 - protein, 13 aa, Guillardia theta
DELNYLIYRYLLE

SEQ ID NO: 22 - protein, 13 aa, Paramecium tetraurelia
EELNYLIYTYFLE

SEQ ID NO: 23 - protein, 13 aa, Arabidopsis thaliana
VELNFLVFRYLQE

SEQ ID NO: 24 - protein, 13 aa, Glycine max
TELNYLVFRYLHE

SEQ ID NO: 25 - protein, 13 aa, Marchantia polymorpha
QELNFLIFRYLHE

SEQ ID NO: 26 - protein, 13 aa, Chlamydomonas reinhardtii
DVVNYLIYRYLQE

SEQ ID NO: 27 - protein, 13 aa, Chlamydomonas eustigma
DVINYLVMRYLQE

SEQ ID NO: 28 - protein, 13 aa, Grifola frondosa
DEVNCLIYAYFQD

SEQ ID NO: 29 - protein, 13 aa, Helobdella robusta
DEVNYLVYRYLIE

SEQ ID NO: 30 - protein, 13 aa, Tetranychus urticae
DEVNYLVYRYLQE

SEQ ID NO: 31 - protein, 13 aa, Nannochloropsis gaditana
DEVNILVHRYLVE

SEQ ID NO: 32 - protein, 13 aa, Globodera pallida
DEVNLLVYRYLEE

SEQ ID NO: 33 - protein, 13 aa, Ectocarpus siliculosus
EEVNFLVYRYLQE

SEQ ID NO: 34 - protein, 13 aa, Homo sapiens
DEVNFLVYRYLQE

SEQ ID NO: 35 - protein, 13 aa, Hypsibius dujardini
EEVNLLIYQYLIE

SEQ ID NO: 36 - protein, 13 aa, Chytriomyces confervae
KDLNKLIMNYLVI

SEQ ID NO: 37 - protein, 13 aa, Schizosaccharomyces pombe
SDVNSLILDYLVI

SEQ ID NO: 38 - protein, 13 aa, Drosophila sechellia
ADLNRLVMNYLVT

SEQ ID NO: 39 - protein, 13 aa, Homo sapiens
ADMNRLIMNYLVT

SEQ ID NO: 40 - protein, 13 aa, Marchantia polymorpha
EDMNKLIMDFFLT

SEQ ID NO: 41 - protein, 13 aa, Zea mays
EDMNRLVMNFLVT

SEQ ID NO: 42 - protein, 13 aa, Arabidopsis thaliana
EDMNTLVMNFLVT

SEQ ID NO: 43 - protein, 13 aa, Amborella trichopoda
EDMNKLVMNFLVT

SEQ ID NO: 44 - protein, 13 aa, Arabidopsis thaliana
EDMNRLVMNFLVV

SEQ ID NO: 45 - protein, 13 aa, Yarrowia lipolytica
ADLNSIVMDYLIV

SEQ ID NO: 46 - protein, 13 aa, Ananas comosus
KDLNLVVLRYLLD

SEQ ID NO: 47 - protein, 13 aa, Saccharomyces cerevisiae
SDLNRIVLEYLNK

SEQ ID NO: 48 - protein, 13 aa, Klebsormidium nitens
EDLNRIVLSYLKK

SEQ ID NO: 49 - protein, 13 aa, Arabidopsis thaliana
RLLSALICEYLDW

SEQ ID NO: 50 - protein, 13 aa, Drosophila melanogaster
KLINQMIMEFLDW

SEQ ID NO: 51 - protein, 13 aa, Schizosaccharomyces pombe
SLLNSYIYDYLIK

SEQ ID NO: 52 - protein, 13 aa, Yarrowia lipolytica
ELLNAYIYDYLLK

SEQ ID NO: 53 - protein, 13 aa, Saccharomyces cerevisiae
QLLYAHIYNYLIK

SEQ ID NO: 54 - protein, 13 aa, Schizosaccharomyces pombe
ESLDYYIYDYFVK

SEQ ID NO: 55 - protein, 13 aa, Pyrus ussuriensis
NALDFYIYNYMLK

SEQ ID NO: 56 - protein, 13 aa, Arabidopsis thaliana
KMLDVYIYDYLVK

SEQ ID NO: 57 - protein, 13 aa, Zea mays
KMLDVYIYDYFVK

SEQ ID NO: 58 - protein, 13 aa, Marchantia polymorpha
KMLDVYIYDYLMK

SEQ ID NO: 59 - protein, 13 aa, Oryza sativa
KMLDVYIYDYLLK

SEQ ID NO: 60 - protein, 13 aa, Arabidopsis thaliana
KMLDVYIHDYLVK

SEQ ID NO: 61 - protein, 13 aa, Selaginella moellendorffii
KMLDVYIHDYLTK

SEQ ID NO: 62 - protein, 13 aa, Bursaphelenchus xylophilus
EELNSALAEYLQR

SEQ ID NO: 63 - protein, 13 aa, Homo sapiens
DELNRAIADYLRS

SEQ ID NO: 64 - protein, 13 aa, Syphacia muris
QRVDRLIIDYLLR

SEQ ID NO: 65 - protein, 13 aa, Caenorhabditis elegans
ERIDSLIYGYLRR

SEQ ID NO: 66 - protein, 13 aa, Schizosaccharomyces pombe
VRLNRLVADYMMA

SEQ ID NO: 67 - protein, 13 aa, Mus musculus
SELLGLVKEYLDF

SEQ ID NO: 68 - protein, 13 aa, Neurospora crassa
ELIQQLVLQFLQH

SEQ ID NO: 69 - protein, 13 aa, Homo sapiens
TMIQKMVSSYLVH

SEQ ID NO: 70 - protein, 13 aa, Candida albicans
QVLNSLILDFLVK

SEQ ID NO: 71 - protein, 13 aa, Schizosaccharomyces pombe
EFLNELISSFLLN

SEQ ID NO: 72 - protein, 13 aa, synthetic construct
KEIIRLILQYLHE

SEQ ID NO: 73 - protein, 13 aa, synthetic construct
EELNRLIMNYLMH

SEQ ID NO: 74 - protein, 13 aa, synthetic construct
EELRNLIADYMQH

SEQ ID NO: 75 - protein, 13 aa, synthetic construct
NMLNVLIYDYLIH

SEQ ID NO: 76 - protein, 13 aa, synthetic construct
KLINQMIMEYLEW

SEQ ID NO: 77 - protein, 13 aa, synthetic construct
XELNRLIXEYLDH

SEQ ID NO: 78 - protein, 13 aa, synthetic construct
DDVNFLVYRYLQE

SEQ ID NO: 79 - protein, 13 aa, synthetic construct
DEVNVLVYRYLQE

SEQ ID NO: 80 - protein, 13 aa, synthetic construct
DEVNFLMYRYLQE

SEQ ID NO: 81 - protein, 13 aa, synthetic construct
DEVNFLVCRYLQE

SEQ ID NO: 82 - protein, 13 aa, synthetic construct
DEVNFLVYQYLQE

SEQ ID NO: 83 - protein, 13 aa, synthetic construct
DEVNFLVYWYLQE

SEQ ID NO: 84 - protein, 13 aa, synthetic construct
KELLFLIRNHLIS

SEQ ID NO: 85 - protein, 13 aa, synthetic construct
KELLLLMRNHLIS

SEQ ID NO: 86 - protein, 13 aa, synthetic construct
KELLLLIQNHLIS

SEQ ID NO: 87 - protein, 13 aa, synthetic construct
KELLLLIRNYLIS

SEQ ID NO: 88 - protein, 13 aa, synthetic construct
HELVFLILQFLDE

SEQ ID NO: 89 - protein, 13 aa, synthetic construct
KELVFLILQFLDE

SEQ ID NO: 90 - protein, 13 aa, synthetic construct
DELVFLILQFLDE

SEQ ID NO: 91 - protein, 13 aa, synthetic construct
EELVFLILQFLDE

SEQ ID NO: 92 - protein, 13 aa, synthetic construct
SELVFLILQFLDE

SEQ ID NO: 93 - protein, 13 aa, synthetic construct
TELVFLILQFLDE

SEQ ID NO: 94 - protein, 13 aa, synthetic construct
NELVFLILQFLDE

SEQ ID NO: 95 - protein, 13 aa, synthetic construct
QELVFLILQFLDE

SEQ ID NO: 96 - protein, 13 aa, synthetic construct
CELVFLILQFLDE

SEQ ID NO: 97 - protein, 13 aa, synthetic construct
GELVFLILQFLDE

SEQ ID NO: 98 - protein, 13 aa, synthetic construct
PELVFLILQFLDE

SEQ ID NO: 99 - protein, 13 aa, synthetic construct
AELVFLILQFLDE

SEQ ID NO: 100 - protein, 13 aa, synthetic construct
IELVFLILQFLDE

SEQ ID NO: 101 - protein, 13 aa, synthetic construct
LELVFLILQFLDE

SEQ ID NO: 102 - protein, 13 aa, synthetic construct
MELVFLILQFLDE

SEQ ID NO: 103 - protein, 13 aa, synthetic construct
FELVFLILQFLDE

SEQ ID NO: 104 - protein, 13 aa, synthetic construct
WELVFLILQFLDE

SEQ ID NO: 105 - protein, 13 aa, synthetic construct
YELVFLILQFLDE

SEQ ID NO: 106 - protein, 13 aa, synthetic construct
VELVFLILQFLDE

SEQ ID NO: 107 - protein, 13 aa, synthetic construct
KEIIRLILQYLHE

SEQ ID NO: 108 - protein, 13 aa, synthetic construct
EELNRLIMNYLMH

SEQ ID NO: 109 - protein, 13 aa, synthetic construct
EELNRLIADYMQH

SEQ ID NO: 110 - protein, 13 aa, synthetic construct
NMLNVLIYDYLIH

SEQ ID NO: 111 - protein, 13 aa, synthetic construct
KLINQMIMEYLEW

SEQ ID NO: 112 - skipped

SEQ ID NO: 113 - protein, 13 aa, synthetic construct
RELVHLILQFLDE

SEQ ID NO: 114 - protein, 13 aa, synthetic construct
RELVDLILQFLDE

SEQ ID NO: 115 - protein, 13 aa, synthetic construct
RELVELILQFLDE

SEQ ID NO: 116 - protein, 13 aa, synthetic construct
RELVTLILQFLDE

SEQ ID NO: 117 - protein, 13 aa, synthetic construct
RELVNLILQFLDE

SEQ ID NO: 118 - protein, 13 aa, synthetic construct
RELVQLILQFLDE

SEQ ID NO: 119 - protein, 13 aa, synthetic construct
RELVCLILQFLDE

SEQ ID NO: 120 - protein, 13 aa, synthetic construct
RELVGLILQFLDE

SEQ ID NO: 121 - protein, 13 aa, synthetic construct
RELVALILQFLDE

SEQ ID NO: 122 - protein, 13 aa, synthetic construct
RELVILILQFLDE

SEQ ID NO: 123 - protein, 13 aa, synthetic construct
RELVLLILQFLDE

SEQ ID NO: 124 - protein, 13 aa, synthetic construct
RELVWLILQFLDE

SEQ ID NO: 125 - protein, 13 aa, synthetic construct
RELVVLILQFLDE

SEQ ID NO: 126 - protein, 36 aa, synthetic construct
RELVFLILQFLDEGGGGSGGGGSRELVFLILQFLDE

SEQ ID NO: 127 - protein, 59 aa, synthetic construct
RELVFLILQFLDEGGGGSGGGGSRELVFLILQFLDEGGGGSGGGGSRELVFLILQFLDE

SEQ ID NO: 128 - protein, 13 aa, synthetic construct
RELVRLILQFLDE

SEQ ID NO: 129 - protein, 13 aa, synthetic construct
AALVFLILQFLAA

SEQ ID NO: 130 - protein, 82 aa, synthetic construct
RELVFLILQFLDEGGGGSGGGGSRELVFLILQFLDEGGGGSGGGGSRELVFLILQFLDEGGGGSGGGGSRELVFLILQFLDE

SEQ ID NO: 131 - protein, 18 aa, synthetic construct
MSSLSRELVFLILQFLDE

(19) United States
(12) Patent Application Publication
(10) Pub. No.: US 2025/0313828 A1
(43) Pub. Date: Oct. 9, 2025

(54) SYNTHETIC GENETIC PLATFORM IN EUKARYOTE CELLS AND METHODS OF USE

(71) Applicant: University of Washington, Seattle, WA (US)

(72) Inventors: Jennifer L. Nemhauser, Seattle, WA (US); Alexander Leydon, Seattle, WA (US); Román Ramos Báez, Seattle, WA (US)

(73) Assignee: University of Washington, Seattle, WA (US)

(21) Appl. No.: 18/862,902

(22) PCT Filed: May 3, 2023
(86) PCT No.: PCT/US2023/066568

§ 371 Date: Nov. 4, 2024

Related U.S. Application Data
(60) Provisional application No. 63/338,637, filed on May 5, 2022.

(51) Int. Cl. C12N 15/10

(52) U.S. Cl. CPC C12N 15/1082; C12N 15/1086

(57) ABSTRACT

Synthetic genetic platforms in eukaryote cells (such as yeast) is described. A representative synthetic genetic platform includes a eukaryote cell genetically modified to express an auxin receptor, an auxin response factor, and a reporter, as well as a fusion construct including a Lis1 Homology (LisH) domain fused to an auxin-responsive protein. The synthetic genetic platforms can be used, for instance, to understand developmental and pathological LisH domain variants, and to test bioactive molecules for LisH domain activity.

### CROSS-REFERENCE TO RELATED APPLICATIONS

This is the U.S. National Phase Application based on International Patent Application No. PCT/US23/66568, filed on May 3, 2023, which claims priority to and the benefit of the earlier filing of U.S. Provisional Application No. 63/338,637, filed on May 5, 2022, which is incorporated by reference herein in its entirety.

### STATEMENT REGARDING FEDERALLY SPONSORED RESEARCH OR DEVELOPMENT

This invention was made with government support under Grant No. 5 R01 GM 107084, awarded by the National Institutes of Health (NIH). The government has certain rights in the invention.

### INCORPORATION BY REFERENCE OF SEQUENCE LISTING

A computer readable text file, entitled “W149-0030US_SeqList.xml” created on or about Nov. 4, 2024, with a file size of 118,784 bytes, contains the Sequence Listing for this application and is hereby incorporated by reference in its entirety.

### FIELD OF THE DISCLOSURE

The current disclosure describes a synthetic genetic platform in yeast. The synthetic genetic platform can be used to understand developmental and pathological Lis1 Homology (LisH) domain variants and/or to test bioactive molecules for LisH domain activity.

### BACKGROUND OF THE DISCLOSURE

Transcriptional control is required for life, and dynamic gene expression creates complexity in development, behavior, and ultimately evolutionary success. Transcriptional repression is essential to dynamic spatiotemporal gene expression and is enacted through a diverse array of mechanisms. Interference with repression leads to developmental defects and cancer. Transcriptional repression is controlled in part by a group of proteins known as corepressors that recruit inhibitory machinery to DNA-binding transcription factors to repress transcription. Corepressor protein families are found throughout all eukaryotes, including: animal SMRT (silencing mediator of retinoic acid and thyroid hormone receptor) and NCoR (nuclear receptor corepressor) complexes, yeast Tup1 and its homologs Drosophila Groucho (Gro) and mammalian transducing-like enhancer (TLE), plant TOPLESS (TPL), TOPLESS-RELATED (TPR1-4), LEUNIG (LUG) and its homolog (LUH), and High Expression of Osmotically responsive genes 15 (HOS15).

Despite knowing the identity of many corepressor proteins involved in establishing transcriptional repression, much is left to uncover about how these complexes integrate input signals to create, sustain, and relieve transcriptional repression.

### SUMMARY OF THE DISCLOSURE

The present disclosure describes synthetic genetic platforms in eukaryote cells, such as yeast and plant cells. The synthetic genetic platform can be used to understand developmental and pathological Lis1 Homology (LisH) domain variants and to test bioactive molecules for LisH domain activity, among myriad other methods.

In particular embodiments, the synthetic genetic platform includes a genetically modified cell that is modified to express one or more platform expression construct, or optionally to express one or more components of the platform from a sequence integrated in the genome of the cell. In particular embodiments, the cell is a yeast cell, such as a Saccharomyces cerevisiae (S. cerevisiae) yeast cell. In particular embodiments, the platform expression construct includes a plasmid encoding an auxin receptor, an auxin response factor, and a reporter. In particular embodiments, the auxin receptor is auxin-signaling F-box 2 (AFB2). In particular embodiments, the auxin response factor is auxin response factor 19 (ARF19). In particular embodiments, the reporter is a fluorescent reporter. In particular embodiments, a fluorescent reporter includes Venus.

Optionally, the genetically modified eukaryotic cell is further modified to express a LisH expression construct. In particular embodiments, the LisH expression construct includes a plasmid encoding a Lis1 Homology domain fused to an auxin-responsive protein. In particular embodiments, the LisH expression construct is a plasmid separate from the platform expression construct. In particular embodiments, the LisH expression construct is on the same plasmid as the platform expression construct.

In particular embodiments, the Lis1 Homology domain includes any Lis1 Homology domain of interest. In particular embodiments, the auxin-responsive protein includes Indoleacetic acid-induced protein 3 (IAA3). In particular embodiments, the activity of bioactive molecules can be screened by contacting the bioactive molecule with the synthetic genetic platform.

Provided herein are genetically modified eukaryotic cells that include, on one or more expression constructs or integrated into the genome of the cell: a sequence encoding an auxin receptor; a sequence encoding an auxin response factor; a sequence encoding a reporter; and a sequence encoding a Lis1 Homology (LisH) domain fused to an auxin-responsive protein. In examples of this embodiment, and in combination with any other embodiment, at least one of the encoding sequences is an element of an expression construct, and optionally the expression construct is in the form of a plasmid. For instance, the first or the second expression construct may be in the form of a plasmid, or both may be—and all elements may be on a single plasmid.

Also provided are embodiments, which can be in combination with any other embodiment, wherein the genetically modified cell is within a library, wherein the library includes genetically modified cells transformed with a library of expression constructs, wherein each expression construct each includes a LisH domain fused to an auxin-responsive protein.

Yet another provided embodiment is a method of determining repression activity, which method includes: identifying or selecting a Lis1 Homology domain (LisH) sequence of interest; synthesizing a plasmid wherein the plasmid includes the LisH sequences of interest fused to an auxin-responsive protein; transforming a eukaryotic cell with the plasmid to create a genetically modified cell; and determining repression activity within the cell.

In examples of genetically engineered eukaryotic cells and method embodiments, and in combination with any other embodiment, the reporter includes a visually detectable protein, such as a fluorescent reporter or a luminescent reporter.

In examples of genetically engineered eukaryotic cells and method embodiments, and in combination with any other embodiment, the LisH sequence of interest is a member of a library of LisH variants; the plasmid is part of a plasmid library of the library of LisH variants; and/or a plurality of cells are transformed with the plasmid library.

In examples of genetically engineered eukaryotic cells and method embodiments, and in combination with any other embodiment, the LisH Domain includes the sequence of any one of SEQ ID NOs: 8-111 or 113-130. In further examples of method embodiments, and in combination with any other embodiment, the LisH Domain includes an alpha-helix including an amino acid sequence XX[I/V/L]XX[Y/I/V/L][I/V/L]XXX[L]XX, wherein “X” can be any amino acid.

Also provided are methods of using any of the genetically modified eukaryotic or engineered cells describe herein. Examples of such assay methods employ genetically modified eukaryotic or engineered cells for genetic mutation testing (e.g., cancer/oncogene testing); for developmental mutation testing; for agricultural mutation testing; and for small molecule testing (for instance, for the development of small molecules into therapeutic compounds, such as drugs).

### BRIEF DESCRIPTION OF THE FIGURES

Some of the drawings submitted herewith may be better understood in color. Applicant considers the color versions of the drawings as part of the original submission and reserves the right to present color images of the drawings in later proceedings. At least some of the drawings submitted herewith were published in Leydon et al. (PNAS 119(41):e2206986119, Oct. 3, 2022; doi.org/10.1073/pnas.2206986119) and the accompanying supplemental materials.

FIGS. 1A-1D. TOPLESS (TPL) Lis1 Homology (LisH) H1 is a very short autonomous repression domain. (FIG. 1A) Repression activity of indicated single and double alanine mutations (with *). (FIG. 1B) Repression activity of HA-tagged H1 constructs. All static genetic components of the AtARCSc (auxin promoter::Venus, ARF19, AFB2), were integrated at the URA3 locus. The H1-HA-IAA3 construct is expressed off of a plasmid carrying the TRP1 prototrophic gene. (FIG. 1C) Repression activity of indicated mutation at H1 position R6 tested by fluorescence flow cytometry. (FIG. 1D) Repression activity of indicated mutation at H1 position F10 tested by fluorescence flow cytometry. (FIGS. 1C, 1D) Protein accumulation was tested by western blot and normalized to yeast PGK1. Protein level was normalized to wild type TPL H1 (bolded outline, and horizontal dotted line) and each data point is color coded in Leydon et al. (2022) on a log 2 scale, with blue and red indicating the lowest and high expression, respectively. (FIGS. 1A-1D) Each panel represents two independent time course flow cytometry experiments of the TPL helices indicated, all fused to IAA3. For all cytometry, every point represents the average fluorescence of at least 10,000 individually measured yeast cells (a.u. —arbitrary units).

FIGS. 2A-2C. The repressive function of the LisH domain is likely ancestral. (FIG. 2A) A cladogram shows relationships in sequence similarity between different H1 sequences representing over one thousand diverse LisH-containing proteins across eukaryotes. The tree (provided herewith on two pages) was constructed using the Maximum Likelihood method (Le & Gascuel, Mol. Biol. Evol. 25, 1307-1320, 2008). Ancestral sequences of interest were inferred at nodes of interest (black dot). Published function and subcellular localization for a representative protein for each sequence is listed in Table 2; (FIG. 2B) H1 sequences (SEQ ID NOs: 4, 8-71) were aligned; in FIG. 2c of Leydon et al., 2022, the residues are colored by their physicochemical class (RASMOL color scheme (Sayle & Milner-White, Trends Biochem. Sci. 20, 374, 1995). Conserved residues in relation to the AtTPL sequence at the top of the alignment are indicated with (·). The consensus sequence for H1 is aligned above the relative conservation rate of different residues along the helix at the bottom of the alignment. (FIG. 2C) Relative repressive function of different H1s using fluorescence flow cytometry. Dashed lines mark the fluorescence of the AtTPL-H1-HA-IAA3 control, and the IAA3 control. Protein accumulation was tested by western blot and normalized to yeast PGK1. Protein level was normalized to wild type TPL H1 (AtTPL, 1st dotted line) and each data point is color coded on a log 2 scale.

FIGS. 3A-3F. Clade logo plots. Each panel represents the residues found in the H1s of proteins across the (FIG. 3A) top 20 most repressive sequences, (FIG. 3B) clade I, (FIG. 3C) Clade II (FIG. 3D) clade Ill, (FIG. 3E) clade IV, (FIG. 3F) and clade V. Taller columns represent more conserved residues. Letters appear longer the more commonly they are found at the specified residue. Letters at well-conserved residues are color coded by their physicochemical class. Logo plots were created with an online tool (Crooks et al., Genome Res. 14, 1188-1190, 2004; weblogo.berkeley.edu/logo.cgi).

FIGS. 4A-4D: LisH domains are important for human disease. (FIG. 4A) and (FIG. 4C) Helical wheel depiction of HsTBL1X and HsDCAF1 H1 sequences colored by their physicochemical class (arrow indicates hydrophobic face) produced by HeliQuest (Gautier et al., Bioinforma. Oxf. Engl. 24, 2101-2102, 2008). Arrows show the mutations found in these loci in the catalog of Somatic Mutations in Cancer (COSMIC) library (Tate et al., Nucleic Acids Res. 47, D941-D947, 2019), and where they occur. (FIG. 4B, FIG. 4D) Effects on protein repressive function of these mutations in HsTBL1 and HsDCAF1 sequences are measured using fluorescence cytometry. Each panel represents two independent time course flow cytometry experiments of the H1s indicated. For all cytometry, every point represents the average fluorescence of at least 10,000 individually measured yeast cells (a.u. —arbitrary units). Protein accumulation was tested by western blot and normalized to yeast PGK1. Protein level was normalized to respective wild type H1 (HsTBL1 or HsDCAF1 respectively, dotted line) and each data point is color coded on a log 2 scale.

FIGS. 5A-5C. The H1 can act as a synthetic repressor domain, for instance in planta. (FIG. 5A) Scheme of representative repression assay of the herein provided platform, including a reporter gene (exemplified with a fluorescent protein-encoding sequence) under control of a promoter responsive to auxin (that is, an auxin responsive element, as exemplified); an auxin receptor; and a Lis1 Homology (LisH) domain fused to an auxin-responsive protein. (FIG. 5B) Scheme of plant repression assay, illustrated with the specific elements used in Example 1. (FIG. 5C) H1 repression assay in Nicotiana benthamiana. Transient expression of indicated H1 constructs in tobacco. Reporter activation was measured in four separate leaf injections (biological replicates) in two days of injection (each panel represents one day of injections). Each leaf was excised at 8 locations and measured for Venus fluorescence using a plate scanner. pDR5:Venus: the synthetic DR5 auxin promoter (Ulmasov et al., Plant Cell 9, 1963-1971, 1997) driving Venus; ARF19: p35S:AtARF19-1×FLAG; Each H1 sequence is identical to the H1-HA-IAA3 construct used in FIGS. 2A-2C except the LxLxL EAR sequence has been mutated to AxAxA as to not recruit endogenous TPL/TPR proteins in N. benthamiana.

FIG. 6 is a graph illustrating that ARC is amenable (and responsive) to addition of small molecule modifiers of activity. Control (DMSO) or test small molecule BC2059 (Tegavivint, “Tega”; concentrations indicated on X-axis) were added to yeast cultures, and the cells were cultured for 4 hours before being measured for fluorescence by flow cytometry. Each data point represents three independent time-course flow cytometry experiments of cells expressing the TPL and TBL helices indicated, all fused to IAA3. Every point represents the average fluorescence of at least 10,000 individually measured yeast cells (a.u.: arbitrary units). Error bars are standard error.

FIGS. 7A and 7B Cancer Variant Detection using the Yeast H1 Platform. (FIG. 7A) This experiment tests the responsiveness of different cancer variant mutations to the small molecule Tegavivint. Here, we cultured yeast carrying either the wild type or mutant TBL1 H1 sequence in the presence or absence of 500 nM Tegatrabetan and measured fluorescence by flow cytometry. Certain cancer mutants were observed to be less sensitive to treatment. This suggests that they may lie in the binding site for Tegatrabetan, and that these variants can be used to screen for small molecules that could be mutation specific. This would be an approach relevant to personalized medicine for a given mutation in a patient. (FIG. 7B) A helical wheel diagram of the TBL1 H1 alpha helix. The solvent facing resides are above the dotted line and are predicted to be the main binding surface for the small molecule tegavivint. The hydrophobic residues are below the dotted line and are known to be critical for LisH homo and heterodimerization as well as transcriptional repression.

### REFERENCE TO SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. In the Sequence Listing:

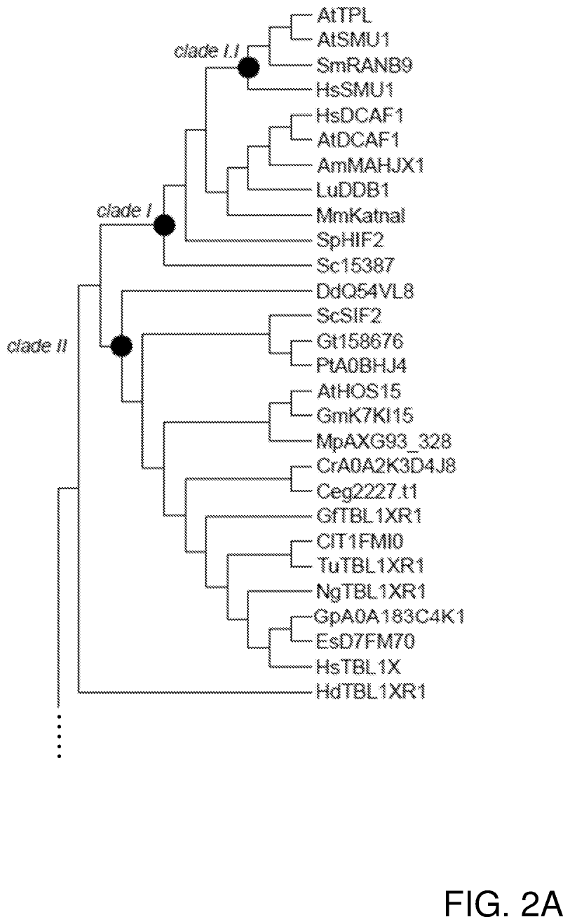

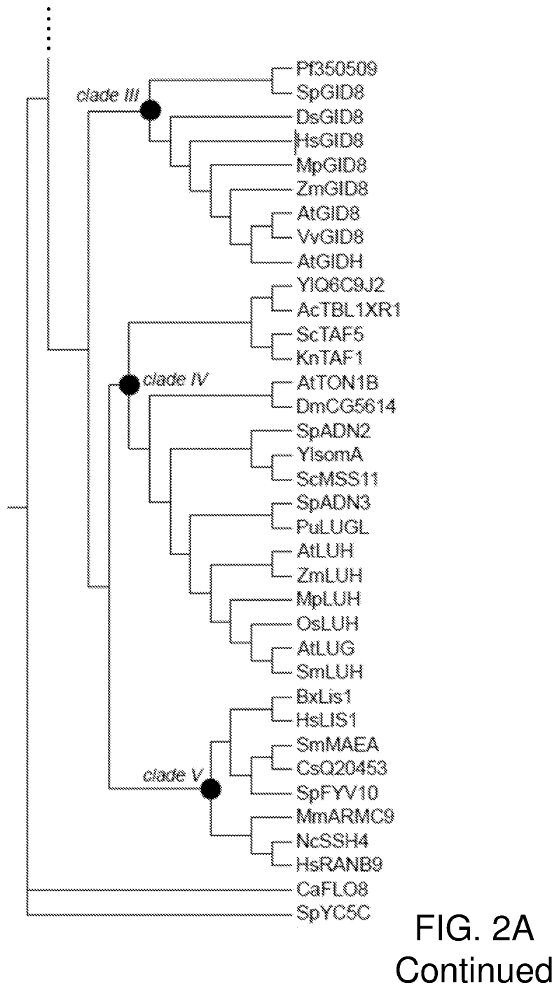

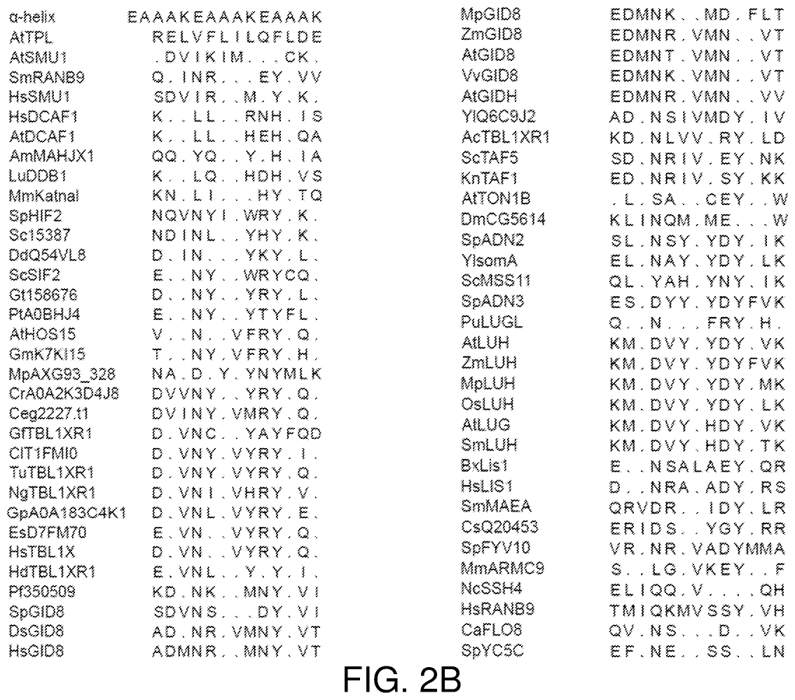

SEQ ID NO: 1 is the amino acid sequence of a representative auxin receptor:
MNYFPDEVIEHVFDFVTSHKDRNAISLVCKSWYKIERYSRQKVFIGNCYAINPERLLRRFPCL
KSLTLKGKPHFADFNLVPHEWGGFVLPWIEALARSRVGLEELRLKRMVVTDESLELLSRSF
VNFKSLVLVSCEGFTTDGLASIAANCRHLRDLDLQENEIDDHRGQWLSCFPDTCTTLVTLNF
ACLEGETNLVALERLVARSPNLKSLKLNRAVPLDALARLMACAPQIVDLGVGSYENDPDSE
SYLKLMAVIKKCTSLRSLSGFLEAAPHCLSAFHPICHNLTSLNLSYAAEIHGSHLIKLIQHCKK
LQRLWILDSIGDKGLEVVASTCKELQELRVFPSDLLGGGNTAVTEEGLVAISAGCPKLHSILY
FCQQMTNAALVTVAKNCPNFIRFRLCILEPNKPDHVTSQPLDEGFGAIVKACKSLRRLSLSG
LLTDQVFLYIGMYANQLEMLSIAFAGDTDKGMLYVLNGCKKMKKLEIRDSPFGDTALLADVS
KYETMRSLWMSSCEVTLSGCKRLAEKAPWLNVEIINENDNNRMEENGHEGRQKVDKLYLY
RTVVGTRMDAPPFVWIL
SEQ ID NO: 2 is the amino acid sequence of a representative auxin response factor:
MKAPSNGFLPSSNEGEKKPINSQLWHACAGPLVSLPPVGSLVVYFPQGHSEQVAASMQKQ
TDFIPNYPNLPSKLICLLHSVTLHADTETDEVYAQMTLQPVNKYDREALLASDMGLKLNRQP
TEFFCKTLTASDTSTHGGFSVPRRAAEKIFPPLDFSMQPPAQEIVAKDLHDTTWTFRHIYRG
QPKRHLLTTGWSVFVSTKRLFAGDSVLFVRDEKSQLMLGIRRANRQTPTLSSSVISSDSMHI
GILAAAAHANANSSPFTIFFNPRASPSEFVVPLAKYNKALYAQVSLGMRFRMMFETEDCGV
RRYMGTVTGISDLDPVRWKGSQWRNLQVGWDESTAGDRPSRVSIWEIEPVITPFYICPPPF
FRPKYPRQPGMPDDELDMENAFKRAMPWMGEDFGMKDAQSSMFPGLSLVQWMSMQQN
NPLSGSATPQLPSALSSFNLPNNFASNDPSKLLNFQSPNLSSANSQFNKPNTVNHISQQMQ
AQPAMVKSQQQQQQQQQQHQHQQQQLQQQQQLQMSQQQVQQQGIYNNGTIAVANQVS
CQSPNQPTGFSQSQLQQQSMLPTGAKMTHQNINSMGNKGLSQMTSFAQEMQFQQQLEM
HNSSQLLRNQQEQSSLHSLQQNLSQNPQQLQMQQQSSKPSPSQQLQLQLLQKLQQQQQ
QQSIPPVSSSLQPQLSALQQTQSHQLQQLLSSQNQQPLAHGNNSFPASTFMQPPQIQVSP
QQQGQMSNKNLVAAGRSHSGHTDGEAPSCSTSPSANNTGHDNVSPTNFLSRNQQQGQA
ASVSASDSVFERASNPVQELYTKTESRISQGMMNMKSAGEHFRFKSAVTDQIDVSTAGTTY
CPDVVGPVQQQQTFPLPSFGFDGDCQSHHPRNNLAFPGNLEAVTSDPLYSQKDFQNLVP
NYGNTPRDIETELSSAAISSQSFGIPSIPFKPGCSNEVGGINDSGIMNGGGLWPNQTQRMR
TYTKVQKRGSVGRSIDVTRYSGYDELRHDLARMFGIEGQLEDPLTSDWKLVYTDHENDILL
VGDDPWEEFVNCVQNIKILSSVEVQQMSLDGDLAAIPTTNQACSETDSGNAWKVHYEDTS
AAASFNR
SEQ ID NO: 3 is the amino acid sequence of a representative PB1 domain sequence:
IYVKVSMDGAPYLRKIDLSCYKGYSELLKALEVMFKFSVGEYFERDGYKGSDFVPTYEDKD
GDWMLIGDVPWEMFICTCKRLRIMKGSEAKGLGCGV
SEQ ID NO: 4 alpha helix control sequence (EAAAK)₃ (SEQ ID NO: 4) was created
based on well-studied synthetic alpha helix linkers
SEQ ID NO: 5 is the amino acid sequence of TPLH1-IAA3 from FIG. 2C of Leydon
et al., 2022:
GRGPGGGHQTSLYKKAGFKM
SEQ ID NO: 6 is the amino acid sequence of TPLH1-1xA-IAA3 from FIG. 2C of
Leydon et al., 2022:
GRGPGGGHQYPYDVPDYAM (of which positions 10-18 are the HA epitope).
SEQ ID NO: 7 is the amino acid sequence of TPLH1-2xA-IAA3 from FIG. 2C of
Leydon et al., 2022:
GRGPGGGHQYPYDVPDYAYPYDVPDYAM (of which positions 10-18 and 19-27 are the two
HA epitopes).

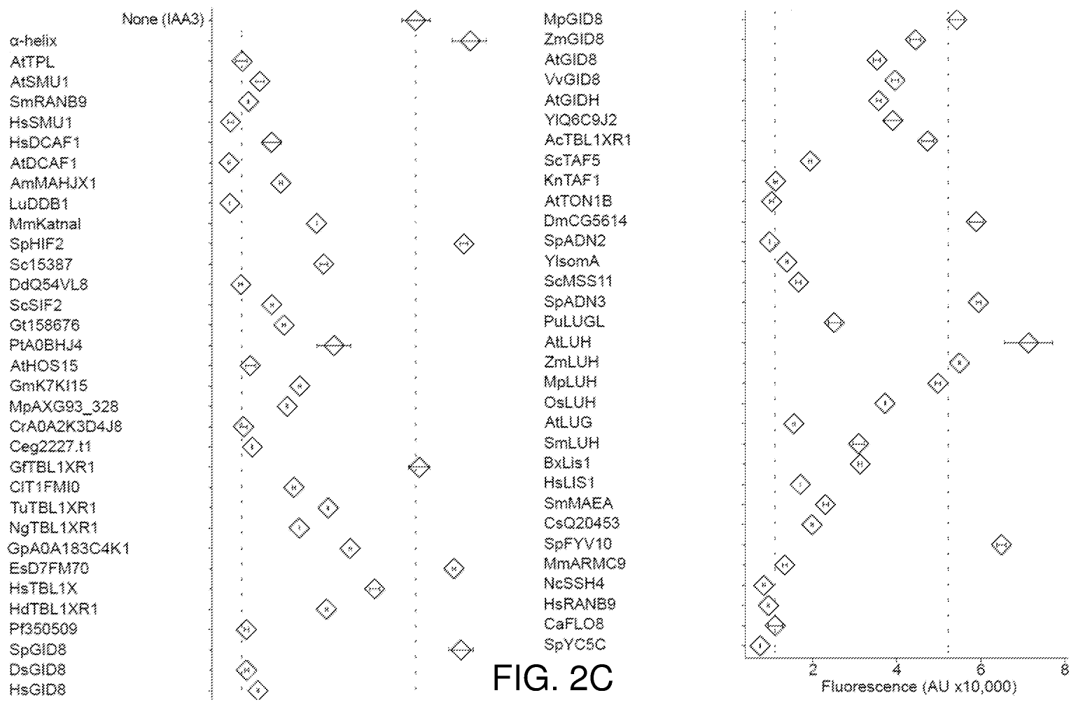

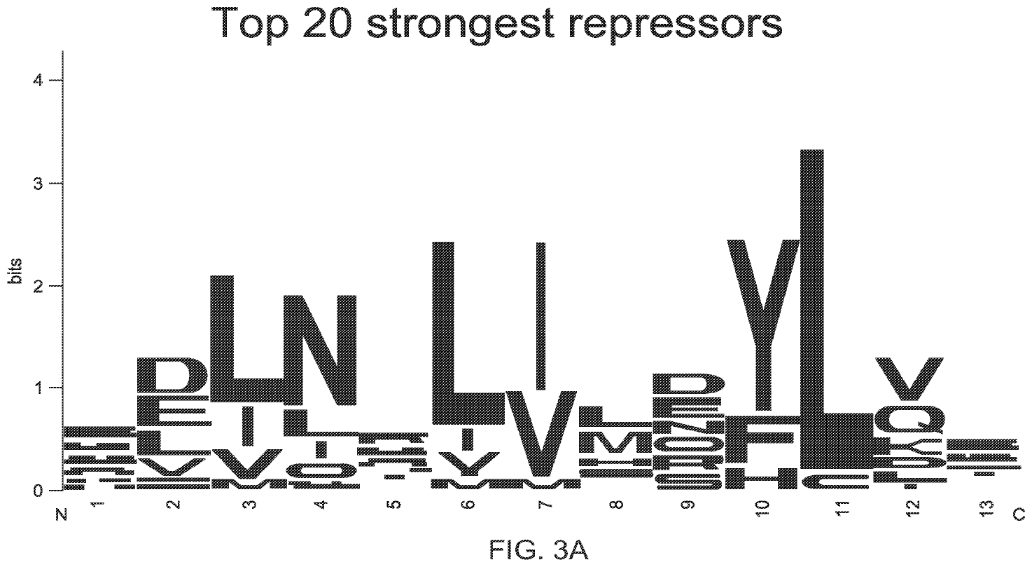

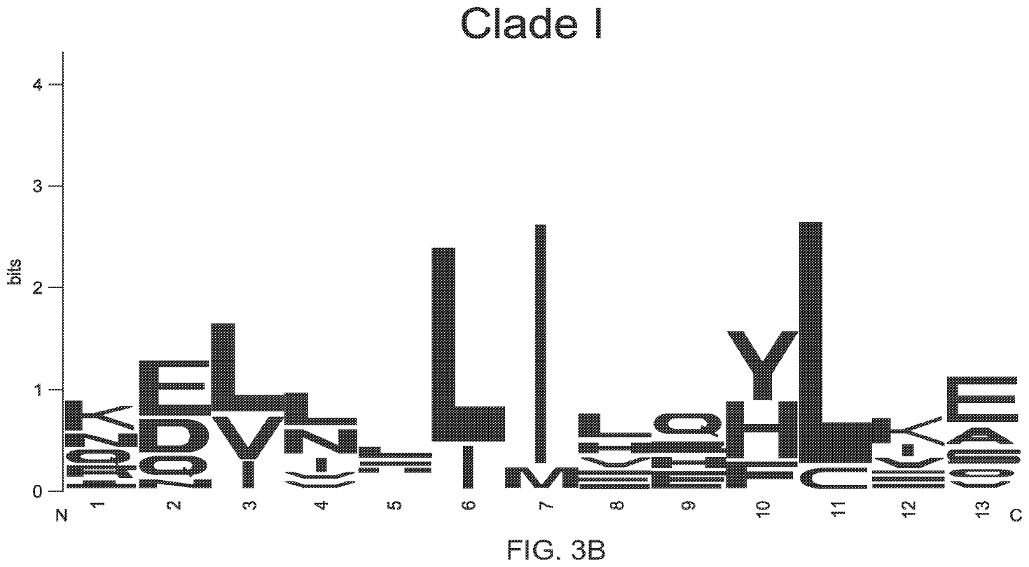

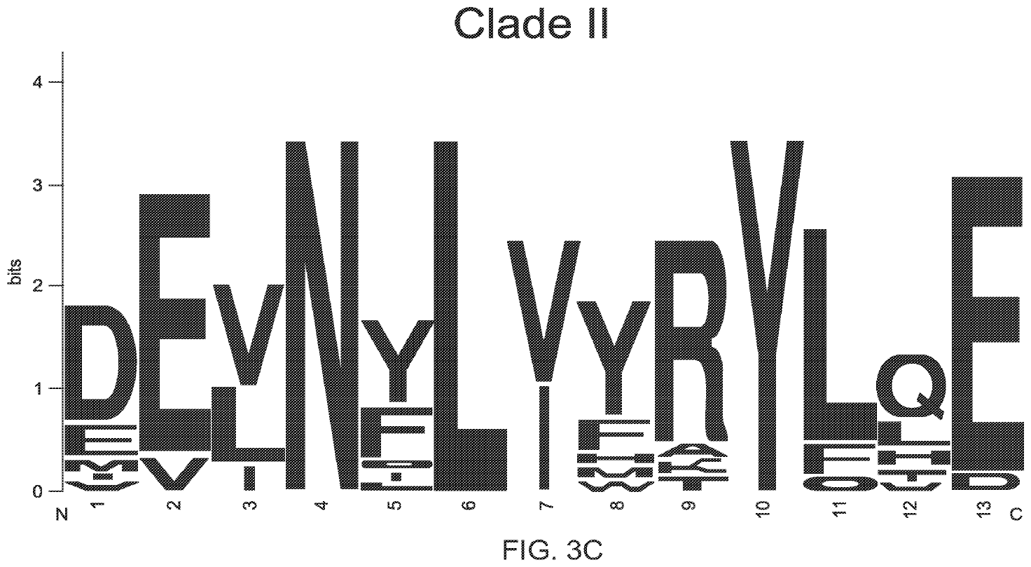

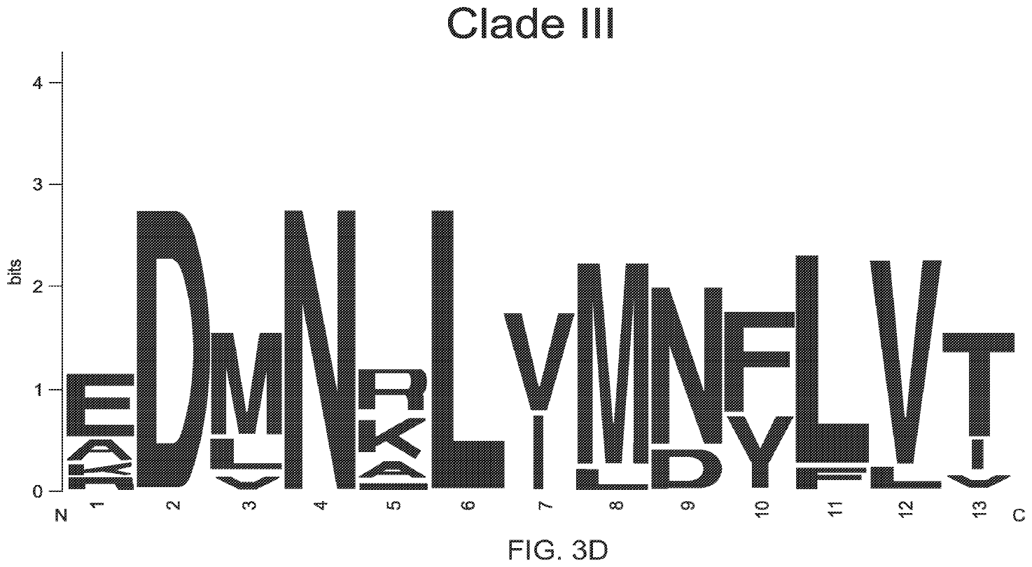

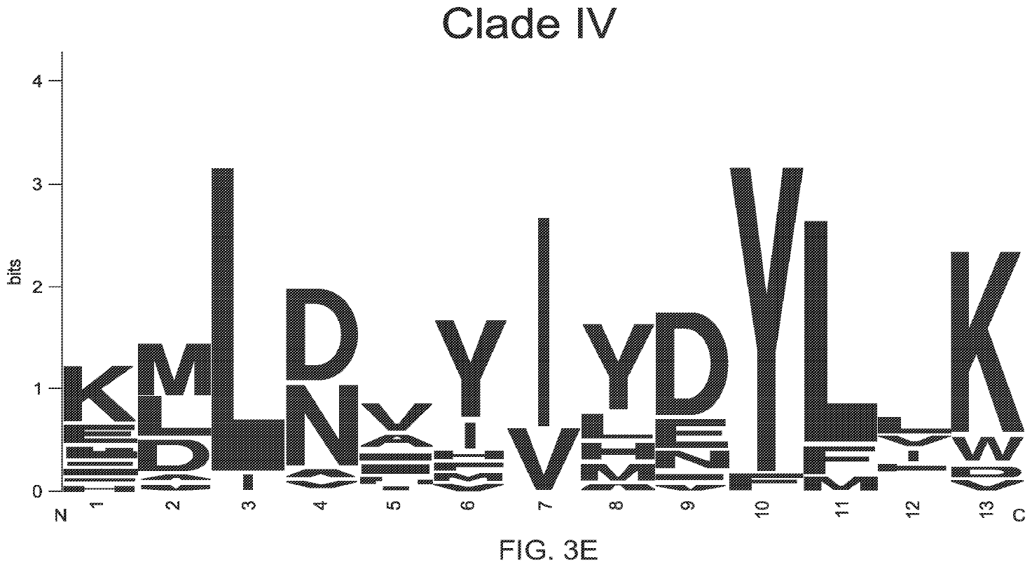

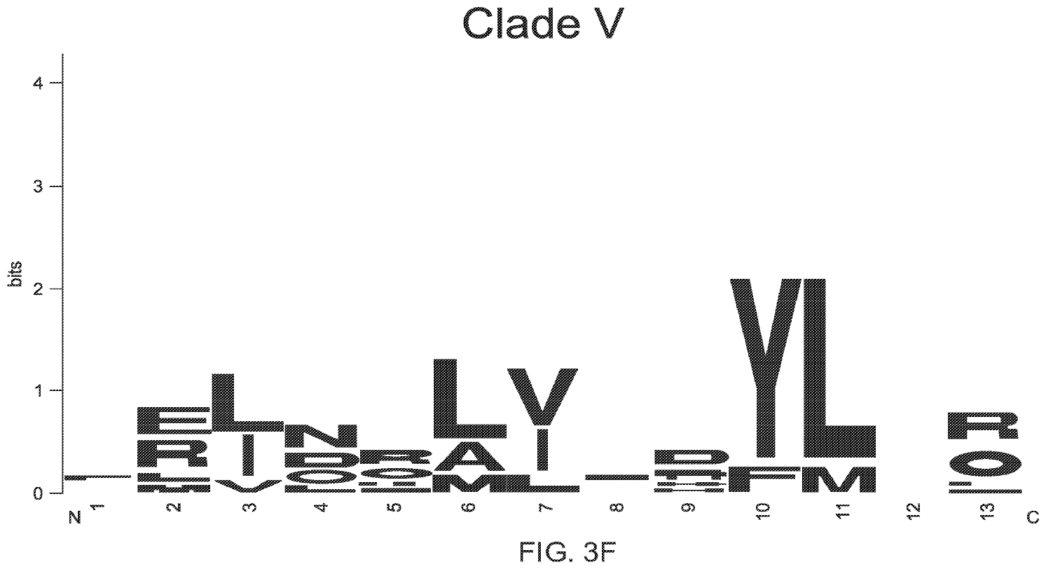

SEQ ID NOs: 8-71 are the amino acid sequences of representative H1 sequences shown in FIG. 3C of Leydon et al., 2022, and Tables 1-3.

SEQ ID NOs: 72-77 are the amino acid sequences of additional H1s, from FIG. 8 of Leydon et al., 2022: KEIIRLILQYLHE (I, SEQ ID NO: 72), EELNRLIMNYLMH (II, SEQ ID NO: 73), EELRNLIADYMQH (III, SEQ ID NO: 74), NMLNVLIYDYLIH (IV, SEQ ID NO: 75), KLINQMIMEYLEW (V, SEQ ID NO: 76), and XELNRLIXEYLDH (Consensus, SEQ ID NO: 77)

SEQ ID NOs: 78-111 and 113-130 are amino acid sequences of representative additional H1 sequences. SEQ ID NO: 112 is left intentionally blank in the Sequence Listing.

SEQ ID NO: 131 is the amino acid sequence of Helix 1; positions 6, 7, 10, 14, 17, and 18 (underlined) are the six amino acids that were mutated to alanine in the context of H1-IAA3: MSSLSRELVFLILQFLDE.

The amino acid sequence of a conserved LisH helix hydrophobic residue consensus pattern is as follows: XX[I/V/L]XX[Y/I/V/L][I/V/L]XXX[L]XX (wherein X can be any amino acid, and the positions in brackets form the hydrophobic face of the helix). This is not included in the Sequence Listing because of its variable structure.

### DETAILED DESCRIPTION

The present disclosure describes synthetic genetic platforms in eukaryotes, including yeast. The synthetic genetic platform can be used to understand developmental and pathological Lis1 Homology (LisH) domain variants and/or to test bioactive molecules for LisH domain activity.

In particular embodiments, the synthetic genetic platform includes a genetically modified cell (such as a yeast cell) that is modified to express a platform expression construct. In particular embodiments, the yeast cell is a Saccharomyces cerevisiae yeast cell. In particular embodiments, the platform expression construct includes a plasmid encoding an auxin receptor, an auxin response factor, and a reporter. In particular embodiments, the auxin receptor is auxin-signaling F-box 2 (AFB2). In particular embodiments, the auxin response factor is auxin response factor 19 (ARF19). In particular embodiments, the reporter is a fluorescent reporter. In particular embodiments, a fluorescent reporter includes Venus.

In particular embodiments, the genetically modified cell is further modified to express a LisH expression construct. In embodiments, the LisH expression construct includes a plasmid encoding a Lis1 Homology domain fused to an auxin-responsive protein. In embodiments, the LisH expression construct is a plasmid separate from the platform expression construct. In embodiments, the LisH expression construct is on the same plasmid as the platform expression construct.

In embodiments, the Lis1 Homology domain includes any Lis1 Homology domain of interest. In embodiments, the auxin-responsive protein includes Indoleacetic acid-induced protein 3 (IAA3). In embodiments, the activity of bioactive molecules can be screened by contacting the bioactive molecule with the synthetic genetic platform.

Provided herein are genetically modified eukaryotic cells that include, on one or more expression constructs or integrated into the genome of the cell: a sequence encoding an auxin receptor; a sequence encoding an auxin response factor; a sequence encoding a reporter; and a sequence encoding a Lis1 Homology (LisH) domain fused to an auxin-responsive protein. In examples of this embodiment, and in combination with any other embodiment, at least one of the encoding sequences is an element of an expression construct, and optionally the expression construct is in the form of a plasmid. For instance, the first or the second expression construct may be in the form of a plasmid, or both may be—and all elements may be on a single plasmid.

In examples of genetically modified eukaryotic cell embodiments, and in combination with any other embodiment, the auxin receptor has one or more of the following characteristics: includes an F-box domain and a leucine-rich repeat (LRR) domain; binds auxin (indole-3-acetic acid); is auxin-signaling F-box 2 (AFB2); includes a sequence having 50% sequence identity to the sequence of SEQ ID NO: 1. In examples of this embodiment, and in combination with any other embodiment, the auxin response factor has one or more of the following characteristics: includes a DNA-binding domain (DBD) and a Phox/Bem1p (PB1) domain; binds the auxin-responsive protein and an auxin response element; is auxin response factor 19 (ARF19); or includes a sequence having 50% identity to the sequence set forth in SEQ ID NO: 2.

In further examples of genetically modified eukaryotic cell embodiments, and in combination with any other embodiment, the auxin response element (when present) includes a sequence upstream of the reporter including a TGTCxx sequence motif. For instance, the TGTCxx sequence motif can include the TGTCTC sequence or TGTCGG sequence.

In further examples of this genetically modified eukaryotic cell embodiment, and in combination with any other embodiment, the reporter includes a visually detectable protein, such as a fluorescent reporter or a luminescent reporter. By way of non-limiting example, the fluorescent reporter may be a Venus fluorescent reporter.

In further examples of genetically modified eukaryotic cell embodiments, and in combination with any other embodiment, the Lis1 Homology domain includes: a cancer variant, a developmental variant, or a Lis1 Homology domain from TOPLESS (TPL), TOPLESS-RELATED (TPR1, TPR2, TPR3, or TPR4), LEUNIG (LUG), LEUNIG homolog (LH), High Expression of Osmotically responsive genes 15 (HOS15), silencing mediator of retinoic acid and thyroid hormone receptor (SMRT), nuclear receptor corepressor (NCoR), Tup1, Groucho (Gro), or transducing-like enhancer (TLE). Additional specific LisH domains are provided in SEQ ID NOs: 8-111 and 113-130, as well as databases described herein.

In further examples of genetically modified eukaryotic cell embodiments, and in combination with any other embodiment, the auxin-responsive protein has one or more of the following characteristics: includes a Phox/Bem1p (PB1) domain and binds the auxin response factor; includes a sequence having 40% sequence identity to the sequence as set forth in SEQ ID NO: 3; or is indoleacetic acid-induced protein 3 (IAA3).

In further examples of genetically modified eukaryotic cell embodiments, and in combination with any other embodiment, the first expression construct further includes or encodes a selection marker; the second expression construct further includes or encodes a selection marker; or both the first and the second expression construct further includes or encodes a selection marker. By way of example, in instances the cell is a yeast cell, and the selection marker includes LEU2, URA3, and/or TRP1.

In further examples of genetically modified eukaryotic cell embodiments, and in combination with any other embodiment, the first expression construct and the second expression construct are on different plasmids; the first and second expression constructs are on the same plasmid; or at least one of the first and second expression constructs is integrated into the genome of the cell.

In further examples of genetically modified eukaryotic cell embodiments, and in combination with any other embodiment, the cell is a metazoan cell, a fungal cell, an algal cell, or a plant cell. For instance, exemplary metazoan cells include a fish cell, an amphibian cell, a reptile cell, a mammalian cell, a bird cell, and an insect cell. In specific examples, the fungal cell is a yeast cell, for instance such as a Saccharomyces cerevisiae yeast cell.

Also provided are embodiments, which can be in combination with any other embodiment, wherein the genetically modified cell is within a library, wherein the library includes genetically modified cells transformed with a library of expression constructs, wherein each expression construct each includes a LisH domain fused to an auxin-responsive protein.

In further examples of genetically modified eukaryotic cell embodiments, and in combination with any other embodiment, the LisH Domain includes the sequence of any one of SEQ ID NOs: 8-111 or 113-130.

In further examples of genetically modified eukaryotic cell embodiments, and in combination with any other embodiment, the LisH Domain includes an alpha-helix including an amino acid sequence XX[I/V/L]XX[Y/I/V/L][I/V/L]XXX[L]XX, wherein “X” can be any amino acid.

In further examples of genetically modified eukaryotic cell embodiments, and in combination with any other embodiment, the genetically modified eukaryotic cell includes: (a) a first expression construct encoding the auxin receptor, the auxin response factor, and the reporter; and a second expression construct encoding the Lis1 Homology (LisH) domain fused to the auxin-responsive protein; (b) a first expression construct encoding at least one of the auxin receptor, the auxin response factor, and/or the reporter; and a second expression construct encoding the Lis1 Homology (LisH) domain fused to the auxin-responsive protein; (c) a first expression construct encoding at least one of the auxin receptor, the auxin response factor, and/or the reporter; and the sequence encoding the Lis1 Homology (LisH) domain fused to the auxin-responsive protein is integrated into the genome of the cell; or (d) at least one of the sequence encoding the auxin receptor, the auxin response factor, and/or the reporter integrated into the genome of the cell; and an expression construct encoding the Lis1 Homology (LisH) domain fused to the auxin-responsive protein.

Yet another provided embodiment is a method of determining repression activity, which method includes: identifying or selecting a Lis1 Homology domain (LisH) sequence of interest; synthesizing a plasmid (e.g., using a versatile genetic assembly system) wherein the plasmid includes the LisH sequences of interest fused to an auxin-responsive protein; transforming a eukaryotic cell with the plasmid to create a genetically modified cell; and determining repression activity within the cell.

In further examples of method embodiments, and in combination with any other embodiment, the LisH sequence includes: a cancer variant; a developmental mutation variant; or the Lis1 Homology domain of TOPLESS (TPL), TOPLESS-RELATED (TPR1, TPR2, TPR3, or TPR4), LEUNIG (LUG), LEUNIG homolog (LH), High Expression of Osmotically responsive genes 15 (HOS15), silencing mediator of retinoic acid and thyroid hormone receptor (SMRT), nuclear receptor corepressor (NCoR), Tup1, Groucho (Gro), or transducing-like enhancer (TLE). In other examples, the LisH domain includes the sequence of any one of SEQ ID NOs: 8-111 or 113-130.

In further examples of method embodiments, and in combination with any other embodiment, the auxin-responsive protein has one or more of the following characteristics: includes a Phox/Bem1p (PB1) domain and binds the auxin response factor; includes a sequence having 40% sequence identity to the sequence set forth in SEQ ID NO: 3; or is indoleacetic acid-induced protein 3 (IAA3).

In further examples of method embodiments, and in combination with any other embodiment, the cell expresses an auxin receptor, an auxin response factor, and a reporter. By way of example, the auxin receptor has one or more of the following characteristics: includes an F-box domain and a leucine-rich repeat (LRR) domain: the auxin receptor binds auxin (indole-3-acetic acid); is auxin-signaling F-box 2 (AFB2); or includes a sequence having 50% sequence identity to the sequence set forth in SEQ ID NO: 1. In further examples of method embodiments, and in combination with any other embodiment, the auxin response factor has one or more of the following characteristics: includes a DNA-binding domain (DBD) and a Phox/Bem1p (PB1) domain; binds the auxin-responsive protein and an auxin response element; is auxin response factor 19 (ARF19); or includes a sequence having 50% identity to the sequence set forth in SEQ ID NO: 2. In further examples of method embodiments, and in combination with any other embodiment, the auxin response element (when present) includes a sequence upstream of the reporter including a TGTCxx sequence motif. For instance, the TGTCxx sequence motif in some cases includes the TGTCTC sequence or TGTCGG sequence.

In further examples of this genetically modified eukaryotic cell embodiment, and in combination with any other embodiment, the reporter includes a visually detectable protein, such as a fluorescent reporter or a luminescent reporter. By way of non-limiting example, the fluorescent reporter may be a Venus fluorescent reporter.

In further examples of method embodiments, and in combination with any other embodiment, the plasmid further includes: an auxin receptor, an auxin response factor, and a reporter, such that the genetically modified cell expresses the auxin receptor, the auxin response factor, and the reporter. In further examples of method embodiments, and in combination with any other embodiment, the auxin receptor has one or more of the following characteristics: includes an F-box domain and a leucine-rich repeat (LRR) domain; binds auxin (indole-3-acetic acid); is auxin-signaling F-box 2 (AFB2); or includes a sequence having 50% sequence identity to the sequence set forth in SEQ ID NO: 1. In further examples of method embodiments, and in combination with any other embodiment, the auxin response factor has one or more of the following characteristics: includes a DNA-binding domain (DBD) and a Phox/Bem1p (PB1) domain; binds the auxin-responsive protein and an auxin response element; is auxin response factor 19 (ARF19); or includes a sequence having 50% identity to the sequence set forth in SEQ ID NO: 2. In further examples of method embodiments, and in combination with any other embodiment, the auxin response element (when present) includes a sequence upstream of the reporter including a TGTCxx sequence motif. By way of example, the TGTCxx sequence motif may include the TGTCTC sequence or TGTCGG sequence.

In further examples of method embodiments, and in combination with any other embodiment, the cell is a yeast cell, and transforming includes at least one of: suspending the yeast cell in lithium acetate solution and contacting the yeast cell with the plasmid; or contacting the yeast cell with the plasmid and heating the yeast cell. Optionally, the method further includes selecting transformed reporter cells after transforming the cell, for instance using a technique that involves positive selection or negative selection.

In further examples of method embodiments, and in combination with any other embodiment, the method further includes screening a bioactive molecule, wherein the screening includes contacting the transformed cell with the bioactive molecule and determining repression activity. By way of example, the bioactive molecule includes one or more of: a small molecule; a peptide or protein; a natural product; a synthetic bioactive compound; an anti-cancer drug; or the anti-cancer drug BC 2059 (Tegavivint).

In further examples of method embodiments, and in combination with any other embodiment, determining repression activity includes performing one or more of: a transcription-based assay; flow cytometry; a Western blot assay, microscopy, a fluorescence assay, or a luminescence assay.

In further examples of method embodiments, and in combination with any other embodiment, the cell is a metazoan cell, a fungal cell, an algal cell, or a plant cell. Example metazoan cells include a fish cell, an amphibian cell, a reptile cell, a mammalian cell, a bird cell, and an insect cell. By way of example, the fungal cell may be a yeast cell (e.g., a haploid or diploid strain), such as a Saccharomyces cerevisiae yeast cell. In other method embodiments, the cell is a plant cell or a plant protoplast.

In further examples of method embodiments, and in combination with any other embodiment, the cell has been transiently or stably transformed with the plasmid to produce the genetically modified cell.

In further examples of method embodiments, and in combination with any other embodiment, the LisH sequence of interest is a member of a library of LisH variants; the plasmid is part of a plasmid library of the library of LisH variants; and/or a plurality of cells are transformed with the plasmid library.

In further examples of method embodiments, and in combination with any other embodiment, the LisH Domain includes the sequence of any one of SEQ ID NOs: 8-111 or 113-130. In further examples of method embodiments, and in combination with any other embodiment, the LisH Domain includes an alpha-helix including an amino acid sequence XX[I/V/L]XX[Y/I/V/L][I/V/L]XXX[L]XX, wherein “X” can be any amino acid.

Also provided are methods of using any of the genetically modified eukaryotic or engineered cells describe herein. By way of example, such methods of use are assays such as testing assays taking advantage of the repressor activity engineered into the cell, based on the synthetic genetic platform described herein. Examples of such assay methods employ genetically modified eukaryotic or engineered cells for genetic mutation testing, such as cancer mutation (e.g., oncogene) testing.

Additional assay methods include methods of using a genetically modified cell of any of the provided embodiments for developmental mutation testing; for agricultural mutation testing; and for small molecule testing (for instance, for the development of small molecules into therapeutic compounds, such as drugs).

Aspects of the current disclosure are now described with additional details and options as follows: (I) Introduction; (II) Auxin Response Circuit (ARC); (Ill) Genetically Modified Eukaryotic Cells; (IV) Expression Constructs; (V) Selection Markers; (VI) Libraries of Cells; (VII) Methods of Use; (VIII) Kits; (IX) Representative Definitions; (X) Sequence Variants and Characterizations; (XI) Exemplary Embodiments; (XII) Experimental Examples; and (XIII) Closing Paragraphs. These headings do not limit the interpretation of the disclosure and are provided for organizational purposes only.

### (I) INTRODUCTION

Transcriptional control is required for life, and dynamic gene expression creates complexity in development, behavior, and ultimately evolutionary success. Transcriptional repression is essential to dynamic spatiotemporal gene expression and is enacted through a diverse array of mechanisms (Reynolds et al., Development 140, 505-512, 2013; Courey & Jia, Genes Dev. 15, 2786-2796, 2001; Payankaulam et al., Curr. Biol. CB 20, R764-R771, 2010; Perissi et al., Nat. Rev. Genet. 11, 109-123, 2010). Interference with repression leads to developmental defects (Grbavec et al., Eur. J. Biochem. 258, 339-349, 1998; Long et al., Science 312, 1520-1523, 2006), and cancer (Wong et al., Am. J. Clin. Exp. Urol. 2, 169-187, 2014). Transcriptional repression is controlled in part by a group of proteins known as corepressors that recruit inhibitory machinery to DNA-binding transcription factors to repress transcription. Corepressor protein families are found throughout all eukaryotes: animal SMRT (silencing mediator of retinoic acid and thyroid hormone receptor) and NCoR (nuclear receptor corepressor) complexes (Mottis et al., Genes Dev. 27, 819-835, 2013; Oberoi et al., Nat. Struct. Mol. Biol. 18, 177-184, 2011), yeast Tup1 (Keleher et al., Cell 68, 709-719, 1992; Matsumura et al., J. Biol. Chem. 287, 26528-26538, 2012; Tzamarias & Struhl, Nature 369, 758-761, 1994) and its homologs Drosophila Groucho (Gro) and mammalian transducing-like enhancer (TLE) (Agarwal et al., IUBMB Life 67, 472-481, 2015), plant TOPLESS (TPL), TOPLESS-RELATED (TPR1-4), LEUNIG (LUG) and its homolog (LUH), and High Expression of Osmotically responsive genes 15 (HOS15) (Long et al., Science 312, 1520-1523, 2006, Causier et al., Plant Physiol. 158, 423-438, 2012; Lee & Golz, Plant Signal. Behav. 7, 86-92, 2012; Liu & Karmarkar, Trends Plant Sci. 13, 137-144, 2008; Zhu et al., Proc. Natl. Acad. Sci. 105, 4945-4950, 2008; Conner et al., Proc. Natl. Acad. Sci. U.S.A 97, 12902-12907, 2000). Despite knowing the identity of many corepressor proteins involved in establishing transcriptional repression, much is left to uncover about how these complexes integrate input signals to create, sustain, and relieve transcriptional repression.

The plant corepressor families (TPL & TPRs, LUG & LUH, and HOS15) all share a general structural similarity, where the N-terminus contain multimerization interfaces, followed by an unstructured linker domain, and a C-terminal WD40 beta-propeller domain (Lee & Golz, Plant Signal. Behav. 7, 86-92, 2012; Liu & Karmarkar, Trends Plant Sci. 13, 137-144, 2008). While the plant corepressors do not share all N-terminal domains, they do share a Lis1 Homology domain (LisH), which is generally known as a protein multimerization interface (Emes & Ponting, Hum. Mol. Genet. 10, 2813-2820, 2001; Delto et al., Struct. Lond. Engl. 1993 23, 364-373, 2015; Kim et al., Biochem. Biophys. Res. Commun. 449, 202-207, 2014′ Tomaštiková et al., BMC Plant Biol. 12, 83, 2012; Ahn et al., Biochemistry 50, 1359-1367, 2011; Choi et al., Mol. Endocrinol. 22, 1093-1104, 2008; Gerlitz et al., Cell Cycle Georget. Tex 4, 1632-1640, 2005; Sherpa et al., Mol. Cell 81, 2445-2459.e13, 2021; Mateja et al., J. Mol. Biol. 357, 621-631, 2006). In TPL the LisH functions to dimerize TPL/TPRs (Martin-Arevalillo et al., Proc. Natl. Acad. Sci. USA, 2017, doi.org/10.1073/pnas.1703054114; Ke et al., Sci. Adv. 1, e1500107, 2015). The LisH is followed by a C-terminal to LisH (CTLH) domain that binds partner proteins via an Ethylene-responsive element binding factor-associated Amphiphilic Repression motif (EAR; Causier et al., Plant Physiol. 158, 423-438, 2012; Kagale et al., Plant Physiol. 152, 1109-1134, 2010). The N-terminal domain also contains a CT11-RanBPM (CRA) domain, which acts as a second homo-multimerization interface and also folds back over and stabilizes the LisH domain (Martin-Arevalillo et al., Proc. Natl. Acad. Sci. U.S.A, 2017) doi.org/10.1073/pnas.1703054114; Ke et al., Sci. Adv. 1, e1500107, 2015). In previous work the N-terminal domain of TPL was found to contain two distinct repression domains that can each repress transcription, one of which is the LisH domain (Leydon et al., eLife 10, e66739, 2021). It was further narrowed down that the first alpha helical region of the Arabidopsis TPL protein, termed hereafter Helix 1 (H1), was sufficient to repress transcription in yeast (Leydon et al., eLife 10, e66739, 2021). Therefore, while the LisH acts as a self-dimerization interface in TPL, it also encodes an additional repressive function, through an unknown mechanism.

The 33-residue LisH motif is found in many proteins across eukaryotes—currently there are 25,290 unique LisH sequence entries in the SMART protein database (SMART, Simple modular architecture research tool. Smartembl-Heidelbergde). These proteins have a variety of functions—including cytoskeleton-interacting proteins, ubiquitin ligase complexes, and transcriptional regulation. The founding member LIS1 regulates microtubule function and the activity of dynein and is required for proper neurodevelopment (Vallee & Tsai, Genes Dev. 20, 1384-1393, 2006). While LIS1 has been broadly studied in its cytoplasmic context, recent work has also demonstrated a nuclear role in gene expression (Keidar et al., Front. Cell. Neurosci. 13, 2019). Several E3 ubiquitin-ligase components carry LisH domains, such as DDB1-Cul4-associated factor 1 (DCAF1, Zhang et al., Gene 263, 131-140, 2001), which is involved in myriad pathways contributing to development and disease (Schabla et al., J. Mol. Cell Biol. 11, 725-735, 2019). The GID E3 ligase complex is another ubiquitylation complex whose protein constituents are assembled by intermolecular LisH interactions (Sherpa et al., Mol. Cell 81, 2445-2459.e13, 2021). Other LisH-containing proteins have well characterized roles in human health and disease such as the oncogene Transducin-beta like 1 (Guenther et al., Genes Dev. 14, 1048-1057, 2000). TBL1 is a component of the SMRT/NCoR complex (Oberoi et al., Nat. Struct. Mol. Biol. 18, 177-184, 2011), and acts as an exchange factor, facilitating the conversion of a SMRT/NCoR repressed locus into an active transcription site (Perissi et al., Cell 116, 511-526, 2004), and its LisH domain is required for its transcriptional activity (Choi et al., Mol. Endocrinol. 22, 1093-1104, 2008). These are only a fraction of the proteins that carry a LisH domain and serve to highlight the broad diversity in protein functions that have evolved that utilize this short and versatile domain. Understanding conserved function in LisH domains is important to understanding the roles these proteins play in development and disease.

Previously, the auxin response pathway was recapitulated in Saccharomyces cerevisiae (yeast) by transferring individual components of the Arabidopsis auxin nuclear response pathway (Pierre-Jerome et al., Proc. Natl. Acad. Sci. U.S.A 111, 9407-9412, 2014). In this Arabidopsis thaliana Auxin Response Circuit in Saccharomyces cerevisiae (AtARCSc), an auxin-responsive transcription factor (ARF) binds to a promoter driving a fluorescent reporter. The ARF protein activity is repressed by interaction with a full-length Aux/IAA protein fused to the N-terminal domain of TPL. Reporter activation can be quantified after auxin addition by microscopy or flow cytometry (Leydon et al., eLife 10, e66739, 2021; Pierre-Jerome et al., Proc. Natl. Acad. Sci. U.S.A 111, 9407-9412, 2014; Havens et al., Plant Physiol. 160, 135-142, 2012). In this way it is possible to test the direct effect of various mutations in TPL, or other transcriptional repressors, at an orthogonal, synthetic locus in a quantitative manner.

Here, yeast was used to interrogate the origins of the LisH domain's helix 1 (H1) repressive function across eukaryotes. Libraries of H1 sequences were used in the AtARCSc to test the function of TPL-H1 residues that control robust transcriptional repression. A library of H1 sequences from diverse proteins across eukaryotes was then used to test the extent of H1 repressive function across both species and proteins of different annotated functions. Yet another set of libraries allowed for quantification of the effect of somatic cancer mutations on TBL1 and DCAF1 stability and function, helping to connect H1 repressive function to oncogenesis. Finally, H1 sequences were tested for viability as a synthetic protein tag for tunable transcriptional repression in a plant system. These findings uncover the ancestral transcriptional repression ability of the LisH domain, showcase how this system can be used to understand disease states, and can be applied as a transcriptional repressor in synthetic biology.

Thus, provided herein are synthetic genetic platforms in eukaryotic cells, which platforms include an auxin receptor, an auxin response factor, a reporter, and a construct encoding a Lis1 Homology (LisH) domain fused to an auxin-responsive protein. When expressed together, this combination takes advantage of the conserved transcription repressor activity of LisH domains (e.g., having the consensus sequence XX[I/V/L]XX[Y/I/V/L][I/V/L]XXX[L]XX, wherein “X” can be any amino acid) to provide an Auxin Response Circuit-type engineered system capable of application as a transcriptional repressor in myriad synthetic biology applications, including several exemplified herein. Myriad appropriate LisH domains (helixes) are provided herein, including in SEQ ID NOs: 8-111 and 113-130.

It is recognized that any LisH domain sequence which shows repression can be measured in the assays provided herein, and that repression is detectable as an output for modulation, for instance modulation by a therapeutic (small molecule, lead compound, test compound peptide, protein, and so forth) introduced into the test system/assay. So, functional H1s (LisH domain) sequences are those that show some amount of repression in a system described herein. The selection of the H1 sequence for use may be influenced for instance by which species and pathways are being studied or tested, or for which a system modifier (e.g., small molecule, drug, etc.) is being developed. Using a diverse library of LisH domains can rule out cross-reactivity with off target/off-species sequences.

It is recognized that some LisH domains may cause destabilization of the fusion protein, for instance such that the protein level is low and may be insufficient for viable some discovery assays (such as assays useful for small molecule screening, for instance). But even these “destabilizing” or “low level” LisH domains are useful in characterizing or screening for (natural or synthetic) variants that produce protein stability (or decrease protein instability).

Though exemplified herein in yeast and plant cells, it is envisioned that the provided synthetic genetic platforms can similarly be embodied in cells of other organisms, including for instance metazoan cells, fungal cells, algal cell, and plant cells—generally, any eukaryotic cell that can be engineered to include the elements required of the described platform, and which are susceptible to detection of a reporter the expression of which is governed/influenced by an LisH domain fused to an auxin-responsive protein. Beneficially, the platform is expressed in a eukaryotic cell that can be grown in culture (e.g., liquid culture) in a substantially single-cell format. Thus, the synthetic genetic platforms described herein, and methods of using them, are contemplated in cultured metazoan cells, such as fish cells, amphibian cells, reptile cells, mammalian cells, bird cells, or insect cells in suspension culture. Similarly, the synthetic genetic platforms described herein, and methods of using them, in other embodiments are contemplated in cultured fungal cells (such as yeast cell, including for instance Saccharomyces cerevisiae yeast cells), cultured plant cells (such as monocotyledonous or dicotyledonous cells, and including plant protoplast cultures), or cultured algal cells (such as Chlamydomonas cells).

Additional details of various elements, combinations, and embodiments are provided herein.

### (II) AUXIN RESPONSE CIRCUIT (ARC)

Auxin represents a family of plant hormones that control gene expression during many aspects of growth and development (Teale et al., Nat. Rev. Mol. Cell Biol. 7:847-859, 2006). Auxin family hormones, such as the naturally-occurring indole-3-acetic acid (IAA) and the synthetic 1-naphthaleneacetic acid (NAA), bind to the F-box transport inhibitor response 1 (TIR1) & Auxin-Signaling F-Box (AFB) protein family of receptors and promote the interaction of the E3 ubiquitin ligase SCF-TIR1 (a form of Skp1, Cullin and F-box (SCF) complex containing TIR1) and the auxin or IAA (AUX/IAA) transcription repressors. SCF-TIR1 recruits an E2 ubiquitin conjugating enzyme that then polyubiquitinates AUX/IAAs, resulting in rapid degradation by the proteasome. Although all eukaryotes have many forms of SCF in which an F-box protein determines substrate specificity, orthologs of TIR1 and AUX/IAAs are only found in plant species. Thus, the auxin-dependent degradation pathways from plants can be applied, in theory, to other eukaryotic species to induce rapid and reversible depletion of a protein of interest in the presence of auxin.

The yeast auxin response circuit (ARC) relies on the Arabidopsis Auxin Response Factor Transcription factor (ARF) as its DNA-binding transcriptional activator, and the Arabidopsis Aux/IAA proteins which act as an adaptor that connects the TPL repressor to the Transcription Factor. To adapt the repressor to usage in other systems such as native or synthetic yeast, plant, or human promoters, modifications are made to the existing system. The first would be the fusion of the TPL-H1 sequence to an alternate DNA-binding protein, such as a native transcription factor (i.e. GAL4-DBD), or a designer protein such as a TALENS or dCAS9. A second optimization would be the design of a reporter that includes an active promoter in the given tissue/cell, driving the transcriptional expression of an appropriate reporter (i.e., a fluorescent protein or other detectable marker) that is easily detectable by flow cytometry or other appropriate assay. Examples of such modified ARC-based systems are described herein. See also FIG. 14 in U.S. Provisional Application No. 63/338,637.

Though the synthetic genetic platforms and methods are generally described herein with regard to an isolated LisH domain that is fused with an auxin responsive protein, it is also recognized that the fusions will function with longer portions of the protein from which the LisH domain is obtained. For instance, the entire protein from which a LisH domain originates can be used in fusions of the synthetic genetic platforms and methods described herein. Examples of this are provided herein, including the TPL original or the TBL1 original ARCs (see Example 1), which contain have a longer portion of the protein that contains the LisH domain. If a larger portion of the protein which contains the LisH is included as in t fusion with an auxin responsive protein, it will perform in an equivalent way.

### (III) GENETICALLY MODIFIED EUKARYOTIC CELLS

The synthetic genetic platforms described herein (e.g., including expression constructs that encode, collectively an auxin receptor, an auxin response factor, a reporter, and a construct encoding a Lis1 Homology (LisH) domain fused to an auxin-responsive protein) are expressed as heterologous systems in eukaryotic cells.

In general, synthetic genetic platforms as provided herein include four components—an auxin receptor (or equivalent), an auxin response factor (or equivalent), a reporter (to detect perturbations in the system, based on changes in one or more interactions of other components in the system), and a LisH domain fused to an auxin-responsive protein (or fused to an equivalent protein). In various embodiments, each of these components may be provided in the genetically modified (host) eukaryotic cell on one or more autonomously replicating expression construct(s) (such as plasmids), or integrated into a genome of the cell.

In particular embodiments, a genetically modified cell includes a nucleic acid where expression of an encoding sequence in the nucleic acid is regulated by a promoter and/or regulatory elements. A promoter and/or regulatory elements are often introduced at a suitable location relative to a gene/an encoding sequence of interest. For example, a promoter (e.g., a constitutive or an inducible promoter) is often placed 5′ of a transcription start site of a gene of interest. In particular embodiments, a nucleic acid includes a promoter and/or regulatory elements necessary to drive the expression of a gene (e.g., a heterologous gene or an endogenous gene). A promoter can be an endogenous promoter, a heterologous promoter, or a combination thereof. In particular embodiments, a promoter is a constitutive promoter.

In particular embodiments, a cell is genetically engineered to include a gene under the control of an inducible promoter. An inducible promoter is often a nucleic acid sequence that directs the conditional expression of a gene. An inducible promoter can be an endogenous promoter, a heterologous promoter, or a combination thereof. In particular embodiments, an inducible promoter requires the presence of a certain compound, nutrient, amino acid, sugar, peptide, protein or condition (e.g., light, oxygen, heat, cold) to induce gene activity (e.g., transcription). In particular embodiments, an inducible promoter includes one or more repressor elements. In particular embodiments, an inducible promoter including a repressor element requires the absence of a certain compound, nutrient, amino acid, sugar, peptide, protein or condition to induce gene activity (e.g., transcription). Any suitable inducible promoter, system, or operon can be used to regulate the expression of a gene.

Host cells are, in many embodiments, unicellular, either because they are unicellular organisms or they are cells from a multicellular organism but are grown in culture as single cells. Suitable eukaryotic host cells include metazoan cells, fungal cells, algal cells, and plant cells. In more particular examples of metazoan cells, the host cell is a fish cell, an amphibian cell, a reptile cell, a mammalian cell, a bird cell, or an insect cell. In examples of plant cells, the host cell is a tobacco (such as Nicotiana tabacum, Nicotiana edwardsonii, Nicotiana plumbagnifolia, Nicotiana longiflora, or Nicotiana benthamiana) cell, an Arabidopsis cell (i.e., rockcress, thale cress, Arabidopsis thaliana), or cells form any other plant that is susceptible to culturing in plant tissue culture, including in suspension cell tissue culture. In examples of alga cells, the host cell is a Chlamydomonas cell (such as Chlamydomonas reinhardtii), a eukaryotic microalgal cell, or any other alga cells susceptible to genetic manipulation/transformation and propagation in liquid culture.

In embodiments, the genus of a host cell can be Aureobasidium, Brettanomyces, Candida, Cryptococcus, Debaromyces, Hansenula, Kloeckera, Kluyveromyces, Lipomyces, Nadsonia, Phaffia, Pichia, Rhodotorula, Saccharomyces, Schizosaccharomyces, Schwanniomyces, Torulopsis, Trichosporon, Trigonopsis, Yarrowia, or Zygosaccharomyces, among others. In examples of yeast or fungal cells, the host cell may be selected from Aspergillus nidulans, Aspergillus niger, Aspergillus oryzae, Candida albicans, Chlamydomonas reinhardtii, Chrysosporium lucknowense, Fusarium sp., Fusarium gramineum, Fusarium venenatum, Hansenula polymorpha, Kluyveromyces sp., Kluyveromyces lactis, Neurospora crassa, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia tnethanolica, Pichia sp., Saccharomyces boulardii, Saccharomyces cerevisiae, Saccharomyces sp., Trichoderma reesei, Yarrowia lipolytica, Zygosaccharomyces bailii, and the like. Saccharomyces cerevisiae is commonly used yeast in industrial processes, and is used in illustrations herein, but the disclosure is not limited thereto. Other yeast species useful in the present disclosure include but are not limited to Schizosaccharomyces pombe, Hansenula anomala, Candida sphaerica, and Schizosaccharomyces malidevorans.

The system described herein in yeast can readily be modified to function in other eukaryotic cells with several key components. For instance, one modification would be to utilize either a synthetic or natural promoter suitable to a different host cell, to drive a reporter protein (i.e. YFP) that has a well-defined binding site in its promoter for a DNA binding protein. This DNA binding protein could be generic (i.e. dCAS9, TALENS, GAL4-DBD) or it could be a species specific DNA binding protein, as in the Arabidopsis ARF protein. The LisH domain or H1 portion would be fused to this DNA binding domain and would be expressed from its own promoter appropriate to that organism. The reporter could also be an endogenous gene that is targeted by the DNA binding protein; its abundance is then measured after repression (which may optionally be disrupted, for instance in methods intended to determine the impact of such disruption(s)). For example a cell surface protein could be targeted that is amenable to antibody binding and detection via fluorescence flow cytometry (like an immune cell protein such as CD4).

In particular embodiments, a transgenic animal cell includes a genetic modification that renders the animal cell appropriate for use in a method provided herein, for instance by expressing a Lis1 Homology (LisH) domain fused to an auxin-responsive protein, optionally along with one or more of a nucleic acid sequence encoding one or more of an auxin receptor, an auxin response factor, and/or a reporter. Methods for generating transgenic animal cells are known in the art. Transgenic animal cells may be of any nonhuman mammalian, avian, or insect species, including mice or nonhuman primates (NHPs). Cells of sheep, horses, cattle, pigs, goats, dogs, cats, rabbits, chickens, and other rodents such as guinea pigs, hamsters, gerbils, rats, and ferrets are also included, as are cells of fruit flies and other insects susceptible to laboratory manipulation. By way of example, the eukaryotic cell or cell line is derived from an invertebrate of the phylum arthropoda, crustacea, or molluska, is an insect cell or cell line. For instance, the eukaryotic tissue, cell, or cell line of may be selected from the group consisting of: a lepidopteran cell, a drosophila cell.

Another embodiment is a eukaryotic cell, or cell line that includes one or more expression construct (or vector) as described herein (or the vector includes an expression cassette or a vector as described herein), wherein a promoter sequence is operably linked to a nucleotide sequence of interest, wherein the promoter sequence leads to expression of the nucleotide sequence encoding a Lis1 Homology (LisH) domain fused to an auxin-responsive protein, or encoding one or more of an auxin receptor, an auxin response factor, and/or a reporter, whereby the promoter sequence is a heterologous promoter sequence, and/or an exogenous promoter sequence.

A further aspect of the disclosure includes methods of producing a genetically altered plant cell that expresses a Lis1 Homology (LisH) domain fused to an auxin-responsive protein, including introducing a nucleic acid sequence encoding a Lis1 Homology (LisH) domain fused to an auxin-responsive protein into a plant cell or other explant; regenerating the plant cell into a genetically altered (transformed) plant cell; and growing the genetically altered plant cell into a genetically altered plant cell culture with a nucleic acid sequence encoding a Lis1 Homology (LisH) domain fused to an auxin-responsive protein. Also provided are embodiments that include identifying successful introduction of the first nucleic acid sequence by screening or selecting the plant cell as an initial transformed plant cell; then introducing into the selected initial transformed plant cell a second nucleic acid sequence encoding one or more of an auxin receptor, an auxin response factor, and/or a reporter. Optionally, the doubly (twice) transformed cell can be screened or selected from non-transformed (or singly transformed) plant cells. Plant cell transformation may be done using a transformation method selected from the group of particle bombardment (i.e., biolistics, gene gun), Agrobacterium-mediated transformation, Rhizobium-mediated transformation, or protoplast transfection or transformation.

Optionally, the first nucleic acid sequence, the second nucleic acid sequence, or both, is/are introduced into the plant (or other) cell in the form of a vector. In embodiments, the first, second or both nucleic acid sequences are operably linked to a promoter—which may be different or the same promoter. Optionally, the promoter(s) may be one or more of a constitutive promoter, an inducible promoter, a plant genus- or plant species-specific promoter, a leaf (or other plant organ) specific promoter, a mesophyll cell (or other cell type) specific promoter, or a photosynthesis gene (or other metabolism-linked gene) promoter. By way of specific example, a constitutive promoter may be a CaMV35S promoter, a derivative of the CaMV35S promoter, a maize ubiquitin promoter, an actin promoter, a trefoil promoter, a vein mosaic cassava virus promoter, or an A. thaliana UBQ10 promoter.

Though largely referred to herein in the context of naturally occurring eukaryotic cells, also contemplated are embodiments wherein the genetically modified cell (that expresses contains and the synthetic platform) is an engineered cell or engineered cell facsimile. Examples of such include organoids in the case of mammalian cells (see, e.g., Kim et al., Nat Rev Mol Cell Biol. 21(10):571-584, 2020; information available online at hsci.harvard.edu/organoids; Arjmand et al., Regen Eng Transl Med 9(1):83-96, 2023) and regenerated plants (Karki et al., Plant Cell Rep 40(7):1087-1099, 2021); these multicellular instantiations can be employed in the herein described methods and screens.

### (IV) EXPRESSION CONSTRUCTS

The synthetic genetic platforms described herein, which include an auxin receptor, an auxin response factor, a reporter, and a construct encoding a Lis1 Homology (LisH) domain fused to an auxin-responsive protein, are provided as (and/or expressed from) one or more expression construct(s). Generally, the expression construct(s) are designed to express the components of the system in a host eukaryotic cell, as has been described herein. Though persons of skill in the art will be familiar with methods to assemble expression construct(s) and components that can be used, including for different target host cells, exemplary descriptions are provided herein.

In general, synthetic genetic platforms as provided herein include four components—an auxin receptor (or equivalent), an auxin response factor (or equivalent), a reporter (to detect perturbations in the system, based on changes in one or more interactions of other components in the system), and a LisH domain fused to an auxin-responsive protein (or fused to an equivalent protein). In various embodiments, each of these components may be provided in the genetically modified (host) eukaryotic cell on one or more autonomously replicating expression construct(s) (such as plasmids), or integrated into a genome of the cell.

In various embodiments, it is contemplated that genetically modified eukaryotic cell of the disclosure contains: a first expression construct encoding the auxin receptor, the auxin response factor, and the reporter; and a second expression construct encoding the Lis1 Homology (LisH) domain fused to the auxin-responsive protein; or a first expression construct encoding at least one of the auxin receptor, the auxin response factor, and/or the reporter; and a second expression construct encoding the Lis1 Homology (LisH) domain fused to the auxin-responsive protein; or a first expression construct encoding at least one of the auxin receptor, the auxin response factor, and/or the reporter; and the sequence encoding the Lis1 Homology (LisH) domain fused to the auxin-responsive protein is integrated into the genome of the cell; or at least one of the sequence encoding the auxin receptor, the auxin response factor, and/or the reporter integrated into the genome of the cell; and an expression construct encoding the Lis1 Homology (LisH) domain fused to the auxin-responsive protein. Use of the term “first” or “second” and the like, is not intended to limit in any way the item referred to but rather to identify it. With particular regard to an expression construct, “first” and “second” do not indicate the order in which they are introduced to or integrated into a host cell.

Any combination of autonomously replicating or integrated elements of the system is functional. In examples described herein, the format provides that each expression construct was separated and integrated into the genome at a distinct locus. Alternatively, encoding sequences for all of the components of the genetic platform can be included in a single expression construct (e.g., plasmid) that can be maintained autonomously in the cell or integrated into a genome of the cell. In one exemplary embodiment, multiple static elements of the genetic platform are integrated at one location in the genome.

Generally speaking, an expression construct or expression vector means a DNA molecule, such as a plasmid (or similar) nucleotide sequence, that has been generated (engineered) through the arrangement of certain polynucleotide sequence elements, wherein the DNA molecule is operable in a host cell of interest (e.g., capable of expressing a polynucleotide encoding a polypeptide of interest, and/or capable of replicating in the host cell). The elements can include vector sequences, regulatory elements, and a polynucleotide sequence comprising at least one coding region encoding a polypeptide of interest. Although the terms expression vector and expression construct can be used interchangeably to describe a DNA molecule that comprises a polynucleotide sequence encoding a polypeptide of interest, as used herein, an “expression vector” in embodiments will not include a coding sequence for a polypeptide of interest, whereas an “expression construct” will include such coding sequence for a polypeptide of interest.

Vectors (including vectors that can serve to deliver an expression cassette to the genome of a cell for integration) are known in the art for expressing recombinant proteins in host cells, and any of these may be used in the present invention. Such vectors include, e.g., plasmids, cosmids, and phage expression vectors. Vector examples are provided herein, and more will be readily recognized by those of skill in the art.

Specific exemplary expression constructs as provided herein include nucleic acid sequence(s) for one or more of a Lis1 Homology (LisH) domain fused to an auxin-responsive protein an auxin receptor, an auxin response factor, and/or a reporter (any of which may be operably linked to a promoter). These functional components of the described genetic platforms are exemplified herein, and described in more detail below. Optionally, an expression construct may include more than one of these functional components, such as all of an auxin receptor, an auxin response factor, and a reporter on a single expression construct. Also contemplated are embodiments where an expression cassette that encodes one (or more than one) of these functional components has been integrated into the genome of a host cell, rather than being maintained in an autonomously replicating nucleic acid molecule.

Auxin Receptor: Auxin receptors (that is, proteins or protein domains that bind to the plant hormone auxin (e.g., indole-3-acetic acid, IAA) are well known; see, for instance, Mockaitis & Estelle (Annu Rev. Cell Dev Biol. 24:55-80, 2008). For instance, the F-box proteins TIR1, AFB2, and AFB3, function as auxin receptors (Dharmasiri et al., Nature 435:441-445, 2005a). The AFB F-box proteins bind auxins directly, and the formation of the auxin-AFB complex is necessary for the binding of Aux/IAA proteins by the SCF (Kepinski & Leyser, Nature, 435:446-451, 2005). The crystal structure of the TIR1 protein in Arabidopsis in the presence and absence of auxin has been determined, and while the F-box region of the AFB proteins interact with the SCF scaffold protein (ASK1 in Arabidopsis), the C-terminal LRRs form an open pocket. The auxin molecule sits in the proximal end of the pocket and acts as a molecular glue, mediating contact between the AFB protein and the targeted Aux/IAA protein. This binding is likely promoted by van der Waals, hydrophobic, and hydrogen-bonding interactions, and may explain why a number of relatively hydrophobic molecules of approximately the same size and general structure can serve as auxins. See, for instance, Patent Publication No. US2012/0060236.

More generally, an auxin receptor includes an F-box domain and a leucine-rich repeat (LRR) domain. Example auxin receptors will bind auxin (e.g., indole-3-acetic acid), and optionally synthetic equivalents therefore. A specific example auxin receptor is auxin-signaling F-box 2 (AFB2). By way of example, an auxin receptor may have a sequence with at least 50% sequence identity with the sequence as set forth in SEQ ID NO: 1 and maintains binding affinity for an auxin; in additional embodiments, the auxin receptor has at least 60%, at least 70%, at least 75%, or more than 75% sequence identity with the sequence as set forth in SEQ ID NO: 1 and maintains binding affinity for an auxin. More specific embodiments of auxin receptor will have at least 80%, at least 85%, at least 90%, at least 92%, at least 95%, or more than 90% identity with the sequence as set forth in SEQ ID NO: 1 and maintains binding affinity for an auxin.

Auxin Response Factor: Auxin Response Factors (ARF) are transcription factors that bind to the auxin response elements in promoters of early auxin response genes. Generally, they are similar to the Aux/IAA proteins in structure (Ulmasov et al., Plant J, 19:309-319, 1999), and contain an N-terminal DNA-binding domain, an RNA polymerase II interaction domain (Hagen & Guilfoyle, Plant Mol Biol, 49:373-385, 2002), and two dimerization domains similar in structure to domains Ill and IV of the Aux/IAA repressors. The DNA-binding domain recognizes a sequence that consists minimally of a conserved sequence (5′-TGTCTC). This sequence, combined with a secondary constitutive element in some genes (Ulmasov et al., Plant J, 19:309-319, 1995), constitutes the auxin responsive element (ARE), which is necessary and sufficient to confer auxin inducibility to reporter genes. While the Aux/IAA proteins are transcriptional repressors, ARFs can act as transcriptional repressors or activators (Hagen & Guilfoyle, Plant Mol Biol, 49:373-385, 2002). These two groups of proteins are capable of both homo- and heterodimerization freely with one another. In the absence of auxin, a heterodimer consisting of one Aux/IAA repressor and one ARF protein (either a repressor or an activator) is bound at the ARE of an auxin-inducible gene, inhibiting transcription. Upon auxin induction, the Aux/IAA protein of that dimmer is degraded, which allows the formation of a new homo- or heterodimer, effecting changes in gene transcription. See, for instance, Patent Publication No. US2012/0060236.

The degradation of Aux/IAA proteins relies on the SCF complex composed of Skp1, Cullin, and F-box (Gray et al., Genes Dev, 13:1678-1691, 1999). The SCF complex is an E3 ubiquitin ligase involved in several signal transduction pathways, including those for gibberellin and jasmonic acid. Skp1 is a scaffold protein, and interacts with two of the other complex members. Cullin transfers ubiquitin subunits from an E2 ubiquitin conjugating enzyme to a specific target protein, and functions as a heterodimer with a fourth protein, RBX1. The F-box proteins are a diverse family of proteins containing a protein-protein interaction domain which interacts with Skp1 called the F-box, and a variety of C-terminal protein-protein interaction domains which confer target specificity to the complex (leucine rich repeats for the AFB family of F-box proteins (Gagne et al., Proc Nat Acad Sci USA, 99:11519-11524, 2002), although a variety of other domain types are present in other groups of F-box proteins).

In embodiments, the auxin response factor (ARF) contains a DNA-binding domain (DBD) and a Phox/Bem1p (PB1) domain. Beneficially, the ARF binds the auxin-responsive protein and an auxin response element that are selected for use in the same platform. One exemplar ARF is auxin response factor 19 (ARF19). By way of example, the ARF has a sequence with at least 50% identity to the sequence set forth in SEQ ID NO: 2 and maintains auxin responsiveness. In additional embodiments, the ARF has at least 60%, at least 70%, at least 75%, or more than 75% sequence identity with the sequence as set forth in SEQ ID NO: 2 and maintains auxin responsiveness. More specific embodiments of ARF will have at least 80%, at least 85%, at least 90%, at least 92%, at least 95%, or more than 90% identity with the sequence as set forth in SEQ ID NO: 2 and maintains auxin responsiveness.

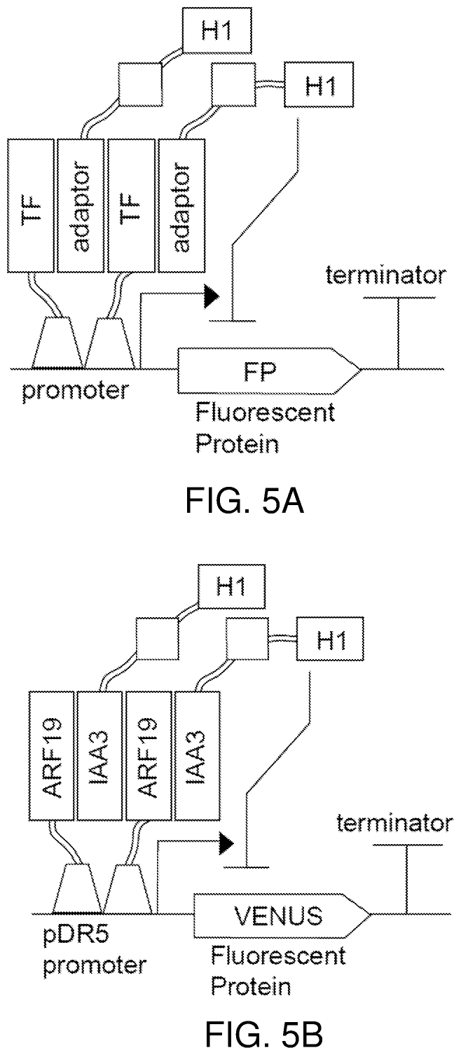

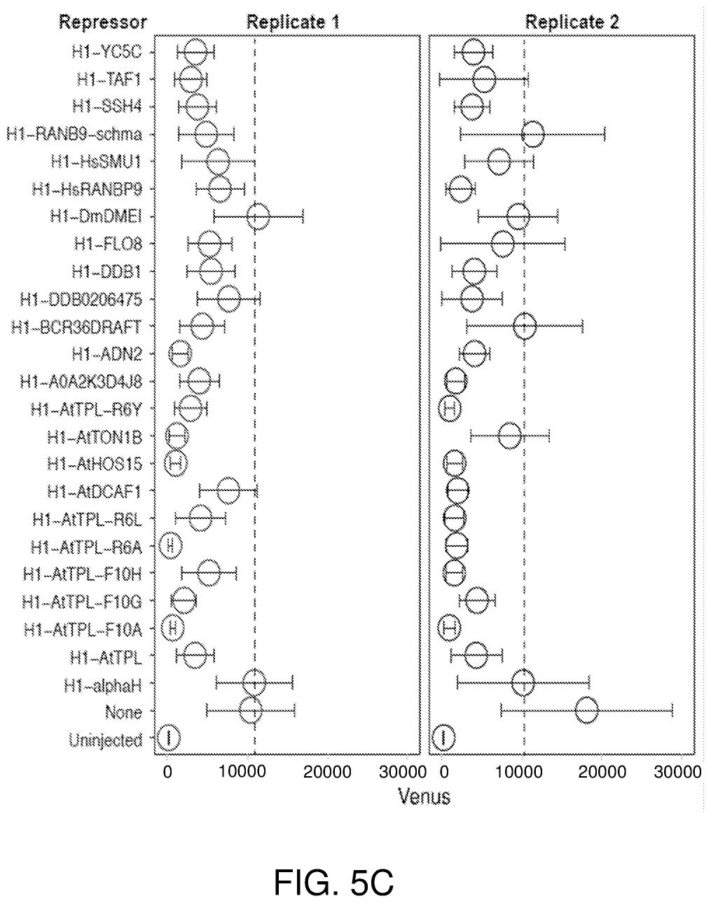

LisH domains: Characterization of H1 sequences is described in Example 1, as is the identification and description of appropriate LisH helical domains for use in the expression constructions, platforms, and methods provided herein. The LisH domain is fused (genetically, to provide a fusion protein) to an auxin responsive protein, as illustrated for instance in FIG. 5A and FIG. 5B.

The 33-residue LIS1 homology (LisH) motif is found in eukaryotic intracellular proteins involved in microtubule dynamics, cell migration, nucleokinesis and chromosome segregation. The LisH motif is likely to possess a conserved protein-binding function and it has been proposed that LisH motifs contribute to the regulation of microtubule dynamics, either by mediating dimerization, or else by binding cytoplasmic dynein heavy chain or microtubules directly. The secondary structure of the LisH domain is predicted to be two alpha-helices. The first alpha helix (H1), the focus of the constructs described herein, is typically 12-18 amino acids long, and contains a solvent exposed face comprised of charged or polar amino acids, and a dimerization face which contains hydrophobic amino acids. Example H1 alpha-helix sequences are provided in SEQ ID NOs: 8-111 or 113-130.

More generally, it is contemplated that any amino acid sequence having the conserved LisH hydrophobic residues pattern XX[I/V/L]XX[Y/I/V/L][I/V/L]XXX[L]XX (where “X” indicates any amino acid) will serve as an effect “LisH” alpha helical domain for fusion to an auxin responsive protein for use in embodiments provided herein.

Specific contemplated Lis1 Homology domains include the Lis1 Homology domain from TOPLESS (TPL), TOPLESS-RELATED (TPR1, TPR2, TPR3, or TPR4), LEUNIG (LUG), LEUNIG homolog (LH), High Expression of Osmotically responsive genes 15 (HOS15), silencing mediator of retinoic acid and thyroid hormone receptor (SMRT), nuclear receptor corepressor (NCoR), Tup1, Groucho (Gro), and transducing-like enhancer (TLE).

Embodiments provided herein allow for use of Lis1 Homology domains obtained from subjects, for instance which have one or more sequence variants (e.g., mutations) compared to the reference (wild type or common) amino acid sequence. Examples of such variants may be variants linked to a disease (which may be causatively or associatively linked, for instance), such as a cancer variant. By way of example, a genetic variant in a LisH domain that is linked to cancer may be viewed as an oncogene.

Also contemplated are other Lis1 homology domain variants, such as developmental variants.

Though the synthetic genetic platforms and methods are generally described herein with regard to an isolated LisH domain that is fused with an auxin responsive protein, it is also recognized that the fusions will function with longer portions of the protein from which the LisH domain is obtained. For instance, the entire protein from which a LisH domain originates can be used in fusions of the synthetic genetic platforms and methods described herein. Examples of this are provided herein, including the TPL original or the TBL1 original ARCs (see Example 1), which contain have a longer portion of the protein that contains the LisH domain. If a larger portion of the protein which contains the LisH is included as in t fusion with an auxin responsive protein, it will perform in an equivalent way.

Auxin-Responsive Protein: Auxin response proteins (ARPs) are known in the art, and will be recognized by those of ordinary skill in the relevant field. Based on the teachings provided herein, known auxin response proteins can be provided as a fusion protein with a LisH domain, for use in the genetic platforms and methods provided herein.

By way of example, an auxin response protein includes a Phox/Bem1p (PB1) domain and binds the auxin response factor. In specific embodiments, the auxin response protein is indoleacetic acid-induced protein 3 (IAA3). In additional embodiments, the auxin response protein includes a sequence having 40% sequence identity to the sequence as set forth in SEQ ID NO: 3, and which maintains the ability to respond to auxin (or a synthetic equivalent thereof). In additional embodiments, the auxin response protein has at least 50%, at least 60%, at least 70%, at least 75%, or more than 75% sequence identity with the sequence as set forth in SEQ ID NO: 3 and maintains the ability to respond to auxin (or a synthetic equivalent thereof). More specific embodiments of auxin response protein will have at least 80%, at least 85%, at least 90%, at least 92%, at least 95%, or more than 90% identity with the sequence as set forth in SEQ ID NO: 2 and maintains the ability to respond to auxin (or a synthetic equivalent thereof).

Reporters/Reporter Genes: A reporter gene is a gene (nucleic acid sequence) that encodes a reporter protein. Any reporter genes/proteins known in the art can be employed in the platform and methods described herein, though it is particularly contemplated that the reporter enables measurement, distinction, and/or separation of cells expressing the reporter based on the fact or amount of that expression. In embodiments, a reporter gene encodes reporter protein that can readily be measured (for instance, an activity of the protein can be measured); optimally, the reporter gene/protein provides a low measurement background. Specific examples of the reporter gene may include a luminescent enzyme gene, a fluorescent protein gene, a color-developing enzyme gene, an active oxygen-generating enzyme gene, a heavy metal-binding protein gene and the like. The reporter (and the gene encoding it) can be selected at least in part based on an apparatus to be used for detecting the resultant signal, such as an activity of the reporter protein.

Exemplary reporter proteins include luminescent and/or fluorescent proteins, such as yellow fluorescent molecules such as SYFP2, Alexa Fluor 532, Citrine, PhiYFP, ZsYellow1, and Venus fluorescent protein (Nagai et al., Nature Biotech. 20:87-90, 2002); red fluorescent molecules such as Texas Red™, mCherry, mRuby, Jred, Alexa Fluor 594, and AsRed2; green fluorescent molecules such as green fluorescent protein (GFP), enhanced green fluorescent protein (EGFP), avGFP, ZsGreen, Alexa Fluor 488, mAzamiGreen, and FITC; orange fluorescent molecules such as Alexa Fluor 546, mOrange, and mKusabira-Orange; blue fluorescent molecules such as Sapphire, mKalamal, EBFP2, and Azurite; cyan fluorescent molecules such as Cerulean and mTurquoise; far red proteins such as mPlum and mNeptune; and cyanine fluorescent molecules such as Cy2, Cy3, Cy5, and Cy7. One of skill in the art will recognize that additional useful variant fluorescent proteins are known and continue to be developed.

There are myriad art-recognized methods for detecting reporters such as those discussed above. By way of example, reporters (of various types) can be detected using a transcription-based assay, flow cytometry, Western blot assay, microscopy, fluorescence assay, or luminescence assay. Detection system(s) are generally tailored for the report being measured.

For usefulness in the provided genetic platforms and methods, the reporter gene is provided operably linked with an auxin responsive element—that is, a genetic sequence that governs/mediates interaction with an auxin response factor. In general, the auxin responsive element includes a sequence upstream of the reporter including at least one repetition of the TGTCxx sequence motif. In specific examples, the TGTCxx sequence motif includes the TGTCTC sequence or TGTCGG sequence.

Additional Expression Construct Characteristics: In addition to the components discussed above, one of skill in the art will recognize that expression constructs for use herein may have additional components, including elements required for expression, replication, and/or maintenance in the host cell. Such elements may be general (useful across multiple platforms), or may be specific for the host cell type or other aspects. The following provides representative elements, though others will be known and recognized by those of skill in the art.

Animal cells in particular can be transformed using viral vectors; generally, this phrase refers to a nucleic acid molecule that includes virus-derived nucleic acid elements that facilitate transfer and expression of non-native nucleic acid molecules within a cell. Adeno-associated viral vector are viral vectors or plasmids containing structural and functional genetic elements, or portions thereof, that are primarily derived from AAV. Retroviral vectors are viral vectors or plasmids containing structural and functional genetic elements, or portions thereof, that are primarily derived from a retrovirus. Lentiviral vectors are viral vectors or plasmids containing structural and functional genetic elements, or portions thereof, that are primarily derived from a lentivirus, and so on. Hybrid vector are vectors including structural and/or functional genetic elements from more than one virus type.

Adenovirus vectors are constructs containing adenovirus sequences sufficient to (a) support packaging of an expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

The typical vector for animal cell transformation is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

Other viral vectors may also be employed. For example, vectors derived from viruses such as vaccinia virus, polioviruses and herpes viruses may be employed. They offer several attractive features for various mammalian cells.

Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In particular embodiments, HIV based vector backbones (i.e., HIV cis-acting sequence elements) can be used.

Elements directing the efficient termination and polyadenylation of a heterologous nucleic acid transcript can increase heterologous gene expression. Transcription termination signals are generally found downstream of the polyadenylation signal. In particular embodiments, vectors include a polyadenylation sequence 3′ of a polynucleotide encoding a polypeptide to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA or SV40 pA. In particular embodiments, a preferred embodiment of an expression construct includes a terminator element. These elements can serve to enhance transcript levels and to minimize read through from the construct into other plasmid sequences.

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript™ plasmid series.

Delivery of Nucleic Acids to Cells: The nucleic acids described herein (including specifically expression constructs, such as vectors or plasmids, that include genetic platform element(s) described herein) can be introduced into cells by techniques known in the art; such techniques may be tailored to be better suited to the specific cell type (e.g., species) into which the nucleic acid(s) are being introduced. The phrase “introducing a nucleic acid into a cell” includes any method for introducing an exogenous nucleic acid molecule into a selected host cell, including transformation, transfection, and transduction. Examples of such methods include calcium phosphate- or calcium chloride-mediated transfection, electroporation, microinjection, particle bombardment, liposome-mediated transfection, transfection using bacterial bacteriophages, transduction using retroviruses or other viruses (such as vaccinia virus or baculovirus of insect cells), cell fusion, chromosome-mediated gene transfer, microcell-mediated gene transfer, spheroplast fusion, cell penetrating peptides, or other methods.

Liposome-mediated delivery methods are approaches using liposomes such as cationic liposomes, for example, cholesterol-based cationic liposomes. The method of using liposomes also includes lipofection, which utilizes the anionic electric properties of the cell surface. Alternatively, liposomes having surface bound with a cell membrane-permeable peptide (e.g., HIV-1 Tat peptide, penetratin, and oligoarginine peptide) can be used.

In particular embodiments, the nucleic acids described herein are stably integrated into the genome of a host cell. In particular embodiments, the nucleic acids are stably maintained in a cell as a separate, episomal segment. Transposons and transposable elements can be used to improve the efficiency of integration, the size of the DNA sequence integrated, and the number of copies of a DNA sequence integrated into a genome. Transposons or transposable elements include a short nucleic acid sequence with terminal repeat sequences upstream and downstream. Active transposons can encode enzymes that facilitate the excision and insertion of nucleic acid into a target DNA sequence.

In particular embodiments, the nucleic acids can incorporate chemical groups that alter the physical characteristics of the nucleic acid and retard degradation in the target cell. As an example, the internucleotide phosphate ester can be optionally substituted with sulfur.

In particular embodiments, nucleic acid constructs can be delivered using cell penetrating peptides. CPPs are short peptides that facilitate cellular uptake of various molecular cargo (from nanosize particles to small chemical molecules and large fragments of DNA). The “cargo” is associated with the peptides either through chemical linkage via covalent bonds or through non-covalent interactions. CPPs are of different sizes, amino acid sequences, and charges but all CPPs have one distinct characteristic: the ability to translocate the plasma membrane and facilitate the delivery of various molecular cargoes intracellularly. CPPs may enter cells through, for example, direct penetration of the membrane, endocytosis-mediated entry, or translocation through the formation of a transitory structure. Examples of CPPs include a transportan peptide (TP), a TP10 peptide, a pVEC peptide, a penetratin peptide, a tat fragment peptide, a signal sequence based peptide, and an amphiphilic model peptide.

### (V) SELECTION MARKERS

The expression constructs disclosed herein may further comprise one or more selection markers, for example, a yeast marker, a yeast antibiotic resistance marker, a yeast auxotrophic marker, a bacterial marker, a bacterial antibiotic resistance marker, a bacterial auxotrophic marker or any combination thereof. The transformed host cells may be grown on selective or nonselective medium. The nature of the marker may be varied widely providing for resistance to a cell growth inhibitor; complementation of. an auxotrophic mutation in the transformed host; morphologic change; or the like.

An expression construct or other recombinant nucleic acid molecule may include a nucleotide sequence encoding a selectable marker. The term or “selectable marker” or “selection marker” refers to a polynucleotide (or encoded polypeptide) that confers a detectable phenotype. A selectable marker in some embodiments encodes a detectable polypeptide, for example, a green fluorescent protein or an enzyme such as luciferase, which, when contacted with an appropriate agent (a particular, wavelength of light or luciferin, respectively) generates a signal that can be detected by eye or using appropriate instrumentation (Giacomin, Plant Sci. 116:59-72, 1996; Scikantha, J. Bacteriol. 178:121, 1996; Gerdes, FEBS Lett. 389:44-47, 1996; see, also, Jefferson, EMBO J. 6:3901-3907, 1987, β-glucuronidase).

In other embodiments, a selectable marker is a molecule that, when present or expressed in a cell, provides a selective advantage (or disadvantage) to the cell containing the marker, for example, the ability to grow in the presence of an agent that otherwise would kill the cell. A selectable marker can provide a means to obtain cells, such as yeast cell, plant cells, or mammalian cells, that express the marker and, therefore, can be useful as a component of a vector of the present disclosure. Examples of selectable markers include, but are not limited to, those that confer antimetabolite resistance, for example, dihydrofolate reductase, which confers resistance to methotrexate (Reiss, Plant Physiol. (Life Sci. Adv.) 13:143-149, 1994); neomycin phosphotransferase, which confers resistance to the aminoglycosides neomycin, kanamycin, and paromycin (Herrera-Estrella, EMBO J. 2:987-995, 1983), hygro, which confers resistance to hygromycin (Marsh, Gene 32:481-485, 1984), trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (White et al., Nucl. Acids Res. 18:1062, 1990; Spencer et al., Theor. Appl. Genet. 79:625-631, 1990), a mutant EPSPV-synthase, which confers glyphosate resistance (Hinchee et al., BioTechnology 91:915-922, 1998), a mutant acetolactate synthase, which confers imidazolione or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants (see, for example, Maliga et al., Methods in Plant Molecular Biology, Cold Spring Harbor Laboratory Press, 1995, page 39).

In particular embodiments, cells expressing a selectable marker can grow in the presence of a selective agent or under a selective growth condition. Examples of selectable markers include antibiotic resistance markers (e.g., chloramphenicol resistance, erythromycin resistance, ampicillin resistance, carbenicillin resistance, kanamycin resistance, spectinomycin resistance, streptomycin resistance, tetracycline resistance, bleomycin resistance, and polymyxin B resistance), markers that complement an essential gene (e.g., diaminopimelic acid auxotrophy (dapD), thymidine auxotrophy (thyA), proline auxotrophy (proBA), glycine auxotrophy (glyA), carbon source auxotrophy (TpiA)), chemical resistance (e.g., tellurite resistance, Fab/for triclosan resistance, bialaphos herbicide resistance, mercury resistance, arsenic resistance), and visual markers (e.g., green fluorescent protein (GFP), yellow fluorescent protein (YFP), other fluorescent proteins, luciferase, β-galactosidase (lacZ)). In particular embodiments, cells may be positively selected that have lost expression of a counter-selectable marker (that is, cells expressing a counter-selectable marker are selected against).

By way of example, in certain yeast embodiments the selection marker includes LEU2, URA3, HIS3, LYS2, and/or TRP1.

### (VI) LIBRARIES

Also provided herein are libraries of cells, which library contains genetically modified cells containing different expression constructs such as constructs each having different H1 (LisH) domain fused to an auxin-responsive protein. One example cell library contains a collection of yeast cells (or plant cells, or insect cells, or metazoan cells such as mammalian cells) transformed with a library of expression constructs, wherein each expression construct comprises a different LisH domain fused to an auxin-responsive protein. The LisH domain library in some instances is a phylogenetic library containing sequences selected based on a LisH alignment from a database, such as the Pfam LisH alignment PF08513 made using the representative protein database (RP15, 1,235 sequences, pfam.xfam.org/family/PF08513). Additional LisH domain libraries include sequences derived from individual subjects, such as for instance domains associated with a disease or condition (e.g., oncogene sequences).

There are also art-recognized libraries that contain annotated LisH domains (or proteins identified as containing one more appropriate domains). See, for instance, the SMART database (available online at smart.embl-heidelberg.de/smart/do_annotation.pl?BLAST=DUMMY&DOMAIN=LisH), which contains 25290 LisH domains in 24886 proteins. Similarly, disease variants can be found in publicly available data bases; by why of example, cancer variants can be found in the COSMIC database (available online at cancer.sanger.ac.uk/cosmic). Synthetic LisH domains, which are engineered rather than identified in nature, can also be used and may be assembled into libraries.

Also contemplated are libraries of expression constructs, for instance plasmid libraries, that are capable of being transformed into cells of expression and use. Such expression construct libraries may provide collections of different LisH domains fused to the same auxin-responsive protein, or collections of other variable elements of the genetic platforms provided herein (e.g., different auxin receptors, different auxin response factors, different reports, or different combinations thereof).

Different libraries can be provided in (or designed for expression in) different host cells, including for instance in a metazoan cell, a fungal cell, an algal cell, or a plant cell. Libraries may be provided in (or designed for expression in) fish cells, amphibian cells, reptile cells, mammalian cells, bird cells, insect cells, or yeast cells (such as Saccharomyces cerevisiae yeast cell).

In addition to libraries of LisH sequences, also provided are libraries of expression cassettes that contain (at least one per cassette) encoding sequence for different LisH sequences; libraries of plasmids or other expression constructs containing such LisH expression cassettes; and libraires of cells (host cells), each of which contains (at lease one per cell) such plasmids. Host cell libraries can be produced in a host cell type of interest, including the host cell types discussed herein.

### (VII) METHODS OF USE

With the provision herein of genetically modified eukaryotic cell that contain expression construct(s) that collectively encode an auxin receptor, an auxin response factor, a reporter; and a Lis1 Homology (LisH) domain fused to an auxin-responsive protein, methods are now enabled for using such transformed cells in repression activity assays. Such assays include, in various embodiments, using the genetically modified cell for cancer mutation testing, for developmental mutation testing, for agricultural mutation testing, and for small molecule testing.

Additional methods that are now enabled include methods of determining repression activity, by identifying (or choosing) a Lis1 Homology domain (LisH) sequence of interest; synthesizing a plasmid wherein the plasmid comprises the LisH sequences of interest fused to an auxin-responsive protein; transforming a eukaryotic cell with the plasmid to create a genetically modified cell; and determining repression activity within the cell.

This platform expands on a transcriptional repression assay in yeast to include domains from many species. Assays for repression and protein stability can be determined to allow for the study of cancer mutations, developmental mutations, agricultural applications, and small molecule testing. Specifically, any mutation found in a LisH domain can be rapidly introduced into the ARC assay. The influence of this variant can tested for its ability to repress the reporter and therefore categorize the effect of mutation on activity. These LisH domain mutations may also modify protein stability, which can be directly measured by standard Western blotting approaches and compared to internal controls. Any of these variants can then be used in small molecule testing to determine interaction and identify LisH-specific and even mutation-specific therapeutics. Further refinement repression strength can be predicted using machine learning approaches, and even small molecules that bind the LisH domain could be predicted.

The technology identifies the effect of variant on transcription for personalized medicine, can be coupled with chemical screening to allow for drug discovery for specific genes and cancer variants, and creates a drug discovery pipeline from structure-activity relationship (SAR) designed molecules. A specific example of screening small molecules using humanized yeast reporter platform and flow cytometry is as follows: Sequences identified from patient DNA sequences harvested from somatic cancers would be identified bioinformatically. These sequence variants would be introduced to the ARC via DNA synthesized into plasmids. These plasmids would be introduced into the ARC reporter strains, and assayed for their ability to repress transcription. These would also be tested to determine their effect on protein abundance. These humanized reporter yeast strains would then be tested against libraries of small molecules (for example libraries of natural products, synthetic bioactive compounds, approved or experimental anti-cancer drugs, and in the case of TBL1 BC2059 and structurally related compounds). Small molecules that show specific or general activity against a given LisH would be prioritized here.

### (VIII) KITS

Also provided are kits useful for carrying out a repressor assay (or repressor inhibition assay), using a synthetic genetic platform described herein. An example of the kit includes one or more of: an expression cassette (genetic platform) with a (heterologous) promoter; and functionally connected thereto, a Lis1 Homology (LisH) domain fused to an auxin-responsive protein; an expression cassettes with a (heterologous) promoter; and functionally connected thereto, one or more of an auxin receptor, an auxin response factor, and/or a reporter; or a cell containing (as an autonomous replicating unit, or integrated into the genome of the cell) at least one of these expression cassettes.

Kits can include instructions, for example written instructions, on how to use the material(s) therein. Material(s) can be, for example, any substance, composition, polynucleotide (e.g., a plasmid or another expression construct), polypeptide, solution, etc., herein or in any patent, patent application publication, reference, or article that is incorporated by reference.

A kit can include one or more of the genetic expression constructs as described herein, or one or more cells containing (e.g., transformed with) such genetic expression construct(s), and optionally additional components such as buffers, reagents, and instructions for carrying out a method described herein. The choice of buffers and reagents will depend on the particular application, e.g., setting of the assay (point-of-care, research, clinical), analyte(s) to be assayed, the detection moiety used, the detection system used, etc.

The kits can also include informational material, which can be descriptive, instructional, marketing, or other material that relates to the methods described herein and/or the use of the devices for the methods described herein. In embodiments, the informational material can include information about production of the device, physical properties of the device, date of expiration, batch or production site information, and so forth.

### (IX) REPRESENTATIVE DEFINITIONS

“Ambient temperature” as used herein refers to the temperature at a location or in a room, or the temperature which surrounds an object under discussion. This term is equivalent to “room temperature” (rt). By way of example, room temperature may be between 65° F. and 78° F. (about 18.3° C. to 25.5° C.); or between 68° F. and 72° F. (about 20° C. to 22.2° C.).

As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

As used herein, the term“downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

The term “endogenous” refers to a molecule (e.g., nucleic acid, gene, RNA, protein) that is naturally occurring or naturally produced in a given cell or cell type.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids or a functional polynucleotide (e.g., siRNA). In particular embodiments, a gene encodes or codes for a protein if the gene is transcribed into mRNA and translation of the mRNA produces the protein in a cell or other biological system. A “gene sequence encoding a protein” includes all nucleotide sequences that are degenerate versions of each other and that code for the same primary amino acid sequence or amino acid sequences of substantially similar form and function.

As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

The term “expression cassette” includes a polynucleotide construct that is generated recombinantly or synthetically and includes regulatory sequences operably linked to a selected polynucleotide to facilitate expression of the selected polynucleotide in a cell. For example, the regulatory sequences can facilitate transcription of the selected polynucleotide in a cell, or transcription and translation of the selected polynucleotide in a cell. Expression of a gene encoding a polypeptide may be upregulated or downregulated by introducing genetic elements such as transcription enhancers or repressors, or translation enhancers or repressors (e.g., modified ribosome binding sites, degradation tags, modified Kozak sequences).

As used herein, the term “genetically modified” or “genetically engineered” refers to the addition of extra genetic material in the form of DNA or RNA into the total genetic material in a cell or modification of the genome of a cell such that the genome contains insertions, deletions, mutations, and/or rearrangements of the genomic DNA after introduction of extra genetic material as compared to a cell that is not genetically modified. The terms “genetically modified cell”, “genetically engineered cell”, “engineered cell”, and “modified cell” are used interchangeably. The term “genetically modified” or “genetically engineered” also refers to multiple genetic modifications, e.g. 2, 3, 4, 5, 6, 7, 8, 9, 10 or more genetic modifications.

In particular embodiments, the term “gene” refers to a nucleic acid sequence (used interchangeably with polynucleotide or nucleotide sequence) that encodes, e.g., a protein such as a marker protein or selection protein, as described herein. This definition includes various sequence polymorphisms, mutations, and/or sequence variants wherein such alterations do not substantially affect the function of the encoded protein. The nucleic acid sequences can include both the full-length nucleic acid sequences as well as non-full-length sequences derived from a full-length protein. The sequences can also include degenerate codons of the native sequence or sequences that may be introduced to provide codon preference in a specific cell. In particular embodiments, the term “gene” may include not only coding sequences but also regulatory regions such as promoters, enhancers, 5′ UTR, 3′UTR, termination regions, and non-coding regions. Gene sequences encoding a molecule can be DNA or RNA that directs the expression of the molecule. These nucleic acid sequences may be a DNA strand sequence that is transcribed into RNA or an RNA sequence that is translated into protein. An essential gene is an endogenous gene (e.g., endogenous to a cell) that produces a polypeptide (e.g., an essential protein) that is necessary for the growth and/or viability of a cell.

A “genetic construct” includes a recombinant nucleic acid, generally recombinant DNA, which has been generated for the purpose of the expression of a specific polynucleotide sequence(s) or is to be used in the construction of other recombinant polynucleotide sequences. In particular embodiments, the term genetic construct includes plasmids and vectors. In particular embodiments, a genetic construct can be circular or linear. Genetic constructs can include, for example, an origin of replication, a multicloning site, a selectable marker, and/or a counter-selectable marker. In particular embodiments, a genetic construct includes an expression cassette. In particular embodiments, an expression cassette (genetic platform) of the disclosure includes: a (heterologous) promoter; and functionally connected thereto, a Lis1 Homology (LisH) domain fused to an auxin-responsive protein. Additional embodiments of expression cassettes of the disclosure include: a (heterologous) promoter; and functionally connected thereto, one or more of an auxin receptor, an auxin response factor, and/or a reporter.

The term “heterologous” refers to a molecule (e.g., nucleic acid, gene, RNA, protein) that originates outside a cell and is introduced into a cell by genetic engineering. In particular embodiments, a heterologous molecule can include sequences that are native to a cell to which the heterologous molecule is introduced; however, the heterologous molecule is synthesized outside the cell and introduced into the cell. The term “exogenous” can be used interchangeably with “heterologous”.

An “isolated” biological component (such as a polynucleotide, polypeptide, or small molecules (e.g., metabolites)) has been substantially separated, produced apart from, or purified away from other biological components in the cell of the organism in which the component originated or was made or naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome; or a chemical compound may be converted to a purified form that is effective or more effective for some use(s) because it is removed from the presence of other components, which may be viewed as contaminants). Polynucleotides and small molecules that have been isolated specifically include nucleic acid molecules and metabolites purified by standard purification methods. The term also embraces biological components (such as nucleic acid molecules) prepared by recombinant expression or production in a host organism or host cell, as well as chemically-synthesized versions, including when they are substantially separated or purified away from other biological components in that product milieu.

As used herein, the term “nucleic acid molecule” refers to a polymeric form of nucleotides, which includes in specific examples both or either of sense and anti-sense strands of RNA, cDNA, genomic DNA, and synthetic forms and mixed polymers of the foregoing. The term includes single- and double-stranded forms of DNA and RNA. A nucleic acid molecule can include either or both naturally occurring and modified nucleotides linked together by naturally occurring and/or non-naturally occurring nucleotide linkages. A nucleotide may be a ribonucleotide, deoxyribonucleotide, or modified form of either. A “polynucleotide” refers to a physical contiguous nucleotide polymer, such as may be comprised in a larger nucleic acid molecule. A nucleic acid molecule is usually at least 10 bases in length, unless otherwise specified. By convention, the nucleotide sequence of a nucleic acid molecule is read from the 5′ to the 3′ end of the molecule. The “complement” of a nucleic acid molecule refers to a polynucleotide having nucleobases that may form base pairs with the nucleobases of the nucleic acid molecule (i.e., A-T/U, and G-C).

Some embodiments include nucleic acids comprising a template DNA that is transcribed into an RNA molecule that comprises a polyribonucleotide that hybridizes to a mRNA molecule. In some examples, the template DNA is the complement of the polynucleotide transcribed into the mRNA molecule, present in the 5′ to 3′ orientation, such that RNA polymerase (which transcribes DNA in the 5′ to 3′ direction) will transcribe the polyribonucleotide from the complement that can hybridize to the mRNA molecule. Unless explicitly stated otherwise, or it is clear to be otherwise from the context, the term “complement” therefore refers to a polynucleotide having nucleobases, from 5′ to 3′, that may form base pairs with the nucleobases of a reference nucleic acid. In some examples, the template DNA is the reverse complement of the polynucleotide transcribed into the mRNA molecule. Thus, unless it is explicitly stated to be otherwise (or it is clear to be otherwise from the context), the “reverse complement” of a polynucleotide refers to the complement in reverse orientation.

As used herein, two polynucleotides are said to exhibit “complete complementarity” when every nucleotide of a polynucleotide read in the 5′ to 3′ direction is complementary to every nucleotide of the other polynucleotide when read in the 5′ to 3′ direction. Similarly, a polynucleotide that is completely reverse complementary to a reference polynucleotide will exhibit a nucleotide sequence where every nucleotide of the polynucleotide read in the 5′ to 3′ direction is complementary to every nucleotide of the reference polynucleotide when read in the 3′ to 5′ direction. These terms and descriptions are recognized in the art and are understood by those of ordinary skill in the art.

Some embodiments of the disclosure include hairpin RNA (hpRNA)-forming RNA molecules. In these hpRNA molecules, both a polyribonucleotide that is substantially identical to the complement or reverse complement of a target ribonucleotide sequence in the target mRNA, and a polyribonucleotide that is substantially the reverse complement thereof, may be found in the same molecule, such that the single-stranded transcribed RNA molecule may “fold over” and hybridize to itself over a region comprising both polyribonucleotides (i.e., in a “stem structure” of the hpRNA).

“Nucleic acid molecules” include all polynucleotides, for example: single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), shRNA (small hairpin RNA), mRNA (messenger RNA), miRNA (micro-RNA), hpRNA (hairpin RNA), tRNA (transfer RNAs, whether charged or discharged with a corresponding acylated amino acid), and cRNA (complementary RNA). The term “deoxyribonucleic acid” (DNA) is inclusive of cDNA, gDNA, and DNA-RNA hybrids. The terms “polynucleotide” and “nucleic acid,” and “fragments” thereof will be understood by those in the art as a term that includes both gDNAs, ribosomal RNAs, transfer RNAs, messenger RNAs, operons, and smaller engineered polynucleotides that encode or may be adapted to encode, peptides, polypeptides, or proteins.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages; for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding polynucleotide,” “structural polynucleotide,” or “structural nucleic acid molecule” refers to a polynucleotide that is ultimately transcribed into an RNA; for example, when placed under the control of appropriate regulatory elements. The boundaries of a coding polynucleotide are determined by a translation start codon at the 5′-terminus and a translation stop codon at the 3′-terminus. Coding polynucleotides include, but are not limited to, gDNA, cDNA, ESTs, and recombinant polynucleotides. As used herein, “transcribed non-coding polyribonucleotide” refers to segments of mRNA molecules such as 5′UTR, 3′UTR, and intron segments that are not translated into a polypeptide. For example, a transcribed non-coding polyribonucleotide may be a polyribonucleotide that natively exists as an intragenic “spacer” in an RNA molecule.

An “oligonucleotide” is a short nucleic acid polymer (a short nucleic acid molecule). Oligonucleotides may be formed by cleavage of longer nucleic acid segments, or by polymerizing individual nucleotide precursors. Automated synthesizers allow the synthesis of oligonucleotides up to several hundred bases in length. Because oligonucleotides may bind to a complementary nucleic acid, they may be used as probes for detecting DNA or RNA. Oligonucleotides composed of DNA (oligodeoxyribonucleotides) may be used in PCR, a technique for the amplification of DNAs. In PCR, the oligonucleotide is typically referred to as a “primer,” which allows a DNA polymerase to extend the oligonucleotide and replicate the complementary strand. Oligonucleotides may also be used in embodiments herein as a probe, either to detect specific polynucleotides or polyribonucleotides as part of an in vitro process, or to detect polynucleotides or polyribonucleotides in a sample from a plant or plant material.

The term “operably linked” refers to polynucleotide sequences or amino acid sequences placed into a functional relationship with one another. For instance, a promoter or enhancer is operably linked to a coding or non-coding sequence if it regulates, or contributes to the modulation of, the transcription of the coding or non-coding sequence. In particular embodiments, regulatory sequences operably linked to a coding sequence are typically contiguous to the coding sequence. However, enhancers can function when separated from a promoter by up to several kilobases or more. Accordingly, some polynucleotide elements may be operably linked but not contiguous. In particular embodiments, a heterologous promoter or heterologous regulatory elements include promoters and regulatory elements that are not normally associated with a particular nucleic acid in nature.

The terms “peptide,” “oligopeptide,” “polypeptide,” “polyprotein,” and “protein” are used interchangeably herein and refer to a polymeric form of amino acids of any length, which can include coded and non-coded amino acids, chemically or biochemically modified or derivatized amino acids, and polypeptides having modified peptide backbones.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

The term “plant” is used in its broadest sense. It includes any species of grass (e.g. turf grass), sedge, rush, ornamental or decorative, crop or cereal, fodder or forage, fruit or vegetable, fruit plant or vegetable plant, flowers, and trees. In particular embodiments, a plant includes: wheat, soybean, maize, barley, millet, rice, turfgrass, cotton, canola, rapeseed, alfalfa, tomato, sugar beet, oats, rye, sorghum, almond, walnut, apple, peanut, strawberry, lettuce, orange, potato, banana, sugarcane, cassava, mango, guava, palm, onions, olives, peppers, tea, yams, cacao, sunflower, asparagus, carrot, coconut, lemon, lime, watermelon, cabbage, cucumber, and grape. A plant part is any part of a plant, tissue of a plant, or cell of a plant. In particular embodiments, a plant or plant part includes: a whole plant, a seedling, cotyledon, meristematic tissue, ground tissue, vascular tissue, dermal tissue, seed, pod, tiller, sprig, leaf, stomata, root, shoot, stem, flower, fruit, pistil, ovaries, pollen, stamen, phloem, xylem, stolon, plug, bulb, tuber, corm, keikis, bud, and blade. “Leaf” and “leaves” refer to a usually flat, green structure of a plant where photosynthesis and transpiration take place and attached to a stem or branch. “Stem” refers to a main ascending axis of a plant. “Seed” refers to a ripened ovule, including the embryo and a casing. In particular embodiments, a cell of the present disclosure includes a plant cell from any plant and/or a plant part described herein.

As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, NY, 1989).

A “plant promoter” refers to a promoter capable of initiating transcription in plant cells. Examples of promoters under developmental control include promoters that preferentially initiate transcription in certain tissues, such as leaves, roots, seeds, fibers, xylem vessels, tracheids, trichomes, or sclerenchyma. Such promoters are referred to as “tissue-preferred”. Promoters which initiate transcription only in certain tissues are referred to as “tissue-specific”. A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most tissue or cell types.

Inducible promoters can be used in some embodiments of the disclosure. See Ward et al., Plant Mol. Biol. 22:361-366, 1993. With an inducible promoter, the rate of transcription increases in response to an inducing agent. Exemplary inducible promoters functional in plant cells include, but are not limited to: Promoters from the ACEI system that respond to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone (Schena et al., Proc. Natl. Acad. Sci. USA 88:0421, 1991).

Exemplary constitutive plant promoters include, but are not limited to: Promoters from plant viruses, such as the 35S promoter from Cauliflower Mosaic Virus (CaMV); promoters from rice actin genes; ubiquitin promoters; pEMU; MAS; maize H3 histone promoter; and the ALS promoter, Xba1/Ncol fragment 5′ to the Brassica napus ALS3 structural gene (or a polynucleotide similar to said Xba1/Ncol fragment) (International PCT Publication No. WO96/30530). A constitutive promoter in some embodiments is selected from the group of a CaMV35S promoter, a derivative of the CaMV35S promoter, a maize ubiquitin promoter, an actin promoter, a trefoil promoter, a vein mosaic cassava virus promoter, or an A. thaliana UBQ10 promoter.

The term “recombinant” refers to a particular DNA or RNA sequence that is the product of various combinations of cloning, restriction, and/or ligation steps resulting in a construct having a structural coding or non-coding sequence distinguishable from homologous sequences found in natural systems. Generally, DNA sequences encoding the structural coding sequence can be assembled from cDNA fragments and short oligonucleotide linkers, or from a series of oligonucleotides, to provide a synthetic gene which is capable of being expressed in a recombinant transcriptional unit. Such sequences can be provided in the form of an open reading frame uninterrupted by internal non-translated sequences, or introns. Genomic DNA including the relevant sequences could also be used. Sequences of non-translated DNA may be present 5′ or 3′ of the open reading frame, where such sequences do not interfere with manipulation or expression of the coding regions. In particular embodiments, the term “recombinant” polynucleotide or nucleic acid refers to one which is not naturally occurring or is made by the artificial combination of two otherwise separated segments of sequence. This artificial combination is often accomplished by either chemical synthesis means, or by the artificial manipulation of isolated segments of nucleic acids, e.g., by genetic engineering techniques. For example, such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

A “recombinant polypeptide” refers to a polypeptide or polyprotein which is not naturally occurring or is made by the artificial combination of two otherwise separated segments of amino acid sequences. This artificial combination may be accomplished by standard techniques of recombinant DNA technology, i.e., a recombinant polypeptide may be encoded by a recombinant polynucleotide. Thus, a recombinant polypeptide is an amino acid sequence encoded by all or a portion of a recombinant polynucleotide.

A “termination region” may be provided by the naturally occurring or endogenous transcriptional termination region of the polynucleotide sequence encoding a protein of the disclosure. Alternatively, the termination region may be derived from a different source. For the most part, the source of the termination region is generally not considered to be critical to the expression of a recombinant protein and a wide variety of termination regions can be employed without adversely affecting expression.

As used herein, the term “transformation” refers to the transfer of one or more polynucleotide(s) into a cell. A cell is “transformed” by or with a polynucleotide when a nucleic acid molecule comprising the polynucleotide is introduced into the cell, and the polynucleotide becomes stably replicated by the cell, either by incorporation of the nucleic acid molecule into the cellular genome, or by episomal replication. Transformation encompasses all techniques by which a nucleic acid molecule can be introduced into such a cell. Examples include, but are not limited to: transfection with viral vectors; transformation with plasmid vectors; electroporation (Fromm et al., Nature 319:791-3, 1986); lipofection (Felgner et al., Proc. Natl. Acad. Sci. USA 84:7413-7, 1987); microinjection (Mueller et al., Cell 15:579-85, 1978); Agrobacterium-mediated transfer (Fraley et al., Proc. Natl. Acad. Sci. USA 80:4803-7, 1983); direct DNA uptake; and microprojectile bombardment (Klein et al., Nature 327:70, 1987).

The term “transgene” refers to an exogenous polynucleotide in the genome of an organism.

“Vectors” include nucleic acid molecules as introduced into a cell, for example, to produce a transformed cell. A vector may include genetic elements that permit it to replicate in the host cell, such as an origin of replication. Examples of vectors include, but are not limited to: a plasmid; cosmid; bacteriophage; or virus that carries exogenous DNA into a cell. A vector may include one or more polynucleotide, including those that encode a Lis1 Homology (LisH) domain fused to an auxin-responsive protein, an auxin receptor, an auxin response factor, and/or selectable marker genes and/or other genetic elements known in the art. A vector may transduce, transform, or infect a cell, thereby causing the cell to express RNA molecules and/or proteins encoded by the vector. A vector optionally includes materials to aid in achieving entry of the nucleic acid molecule into the cell (e.g., a liposome, protein coating, etc.).

### (X) SEQUENCE VARIANTS AND CHARACTERIZATIONS

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W. H. Freeman and Company.

In making such changes, the hydropathic index of amino acids may be considered. The importance of the hydropathic amino acid index in conferring interactive biologic function on a protein is generally understood in the art (Kyte & Doolittle, J. Mol. Biol. 157(1), 105-32, 1982). Each amino acid has been assigned a hydropathic index on the basis of its hydrophobicity and charge characteristics (Kyte & Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. Variants of gene sequences can include codon optimized variants, sequence polymorphisms, splice variants, and/or mutations that do not affect the function of an encoded product to a statistically significant degree. Codon optimization relates to the process of altering a naturally occurring polynucleotide sequence (thereby producing a codon optimized variant) to enhance expression in the target organism, for example, an invertebrate (such as an insect), a plant, a mammal, a fungus, and so forth.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3 M NaCl; 0.2 M NaH₂PO₄; 0.02 M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (XI) EXEMPLARY EMBODIMENTS

1. A genetically modified eukaryotic cell including, on one or more expression constructs or integrated into the genome of the cell: a sequence encoding an auxin receptor; a sequence encoding an auxin response factor; a sequence encoding a reporter; and a sequence encoding a Lis1 Homology (LisH) domain fused to an auxin-responsive protein.

2. The genetically modified cell of embodiment 1, wherein at least one of the encoding sequences is an element of an expression construct, and optionally the expression construct is in the form of a plasmid.

3. The genetically modified cell of embodiment 1, wherein the auxin receptor has one or more of the following characteristics: includes an F-box domain and a leucine-rich repeat (LRR) domain; binds auxin (indole-3-acetic acid); is auxin-signaling F-box 2 (AFB2); includes a sequence having 50% sequence identity to the sequence of SEQ ID NO: 1.

4. The genetically modified cell of embodiment 1, wherein the auxin response factor has one or more of the following characteristics: includes a DNA-binding domain (DBD) and a Phox/Bem1p (PB1) domain; binds the auxin-responsive protein and an auxin response element; is auxin response factor 19 (ARF19); or includes a sequence having 50% identity to the sequence set forth in SEQ ID NO: 2.

5. The genetically modified cell of embodiment 4, wherein the auxin response element (when present) includes a sequence upstream of the reporter including a TGTCxx sequence motif.

6. The genetically modified cell of embodiment 5, wherein the TGTCxx sequence motif includes the TGTCTC sequence or TGTCGG sequence.

7. The genetically modified cell of embodiment 1, wherein the reporter includes a fluorescent reporter or a luminescent reporter.

8. The genetically modified cell of embodiment 7, wherein the fluorescent reporter is a Venus fluorescent reporter.

9. The genetically modified cell of embodiment 2, wherein the second expression construct is in the form of a plasmid.

10. The genetically modified cell of embodiment 1, wherein the Lis1 Homology domain includes: a cancer variant, a developmental variant, or a Lis1 Homology domain from TOPLESS (TPL), TOPLESS-RELATED (TPR1, TPR2, TPR3, or TPR4), LEUNIG (LUG), LEUNIG homolog (LH), High Expression of Osmotically responsive genes 15 (HOS15), silencing mediator of retinoic acid and thyroid hormone receptor (SMRT), nuclear receptor corepressor (NCoR), Tup1, Groucho (Gro), or transducing-like enhancer (TLE).

11. The genetically modified cell of embodiment 1, wherein the auxin-responsive protein has one or more of the following characteristics: includes a Phox/Bem1p (PB1) domain and binds the auxin response factor; includes a sequence having 40% sequence identity to the sequence as set forth in SEQ ID NO: 3; or is indoleacetic acid-induced protein 3 (IAA3).

12. The genetically modified cell of embodiment 2, wherein: the first expression construct further includes or encodes a selection marker; the second expression construct further includes or encodes a selection marker; or both the first and the second expression construct further includes or encodes a selection marker.

13. The genetically modified cell of embodiment 12, wherein the cell is a yeast cell, and the selection marker includes LEU2, URA3, and/or TRP1.

14. The genetically modified cell of embodiment 2, wherein: the first expression construct and the second expression construct are on different plasmids; the first and second expression constructs are on the same plasmid; or at least one of the first and second expression constructs is integrated into the genome of the cell.

15. The genetically modified cell of embodiment 1, wherein the cell is a metazoan cell, a fungal cell, an algal cell, or a plant cell.

16. The genetically modified cell of embodiment 15, wherein the metazoan cell is a fish cell, an amphibian cell, a reptile cell, a mammalian cell, a bird cell, or an insect cell.

17. The genetically modified cell of embodiment 15, wherein the fungal cell is a yeast cell.

18. The genetically modified cell of embodiment 17, wherein the yeast cell is a Saccharomyces cerevisiae yeast cell.

19. The genetically modified cell of embodiment 1, within a library, wherein the library includes genetically modified cells transformed with a library of expression constructs, wherein each expression construct each includes a LisH domain fused to an auxin-responsive protein.

20. The genetically modified cell of any one of embodiments 1-19, wherein the LisH Domain includes the sequence of any one of SEQ ID NOs: 8-111 or 113-130.

21. The genetically modified cell of any one of embodiments 1-19, wherein the LisH Domain includes an alpha-helix including an amino acid sequence XX[I/V/L]XX[Y/I/V/L][I/V/L]XXX[L]XX, wherein “X” can be any amino acid.

22. The genetically modified eukaryotic cell of embodiment 1, including: (a) a first expression construct encoding the auxin receptor, the auxin response factor, and the reporter; and a second expression construct encoding the Lis1 Homology (LisH) domain fused to the auxin-responsive protein; (b) a first expression construct encoding at least one of the auxin receptor, the auxin response factor, and/or the reporter; and a second expression construct encoding the Lis1 Homology (LisH) domain fused to the auxin-responsive protein; (c) a first expression construct encoding at least one of the auxin receptor, the auxin response factor, and/or the reporter; and the sequence encoding the Lis1 Homology (LisH) domain fused to the auxin-responsive protein is integrated into the genome of the cell; or (d) at least one of the sequence encoding the auxin receptor, the auxin response factor, and/or the reporter integrated into the genome of the cell; and an expression construct encoding the Lis1 Homology (LisH) domain fused to the auxin-responsive protein.

23. A method of determining repression activity including: identifying or selecting a Lis1 Homology domain (LisH) sequence of interest; synthesizing a plasmid wherein the plasmid includes the LisH sequences of interest fused to an auxin-responsive protein; transforming a eukaryotic cell with the plasmid to create a genetically modified cell; and determining repression activity within the cell.

24. The method of embodiment 23, wherein the LisH sequence of interest includes: a cancer variant; a developmental mutation variant; or the Lis1 Homology domain of TOPLESS (TPL), TOPLESS-RELATED (TPR1, TPR2, TPR3, or TPR4), LEUNIG (LUG), LEUNIG homolog (LH), High Expression of Osmotically responsive genes 15 (HOS15), silencing mediator of retinoic acid and thyroid hormone receptor (SMRT), nuclear receptor corepressor (NCoR), Tup1, Groucho (Gro), or transducing-like enhancer (TLE).

25. The method of embodiment 23, wherein the auxin-responsive protein has one or more of the following characteristics: includes a Phox/Bem1p (PB1) domain and binds the auxin response factor; includes a sequence having 40% sequence identity to the sequence set forth in SEQ ID NO: 3; or is indoleacetic acid-induced protein 3 (IAA3).

26. The method of embodiment 23, wherein synthesizing a plasmid includes a versatile genetic assembly system.

27. The method of embodiment 23, wherein the cell expresses an auxin receptor, an auxin response factor, and a reporter.

28. The method of embodiment 27, wherein the auxin receptor has one or more of the following characteristics: includes an F-box domain and a leucine-rich repeat (LRR) domain: the auxin receptor binds auxin (indole-3-acetic acid); is auxin-signaling F-box 2 (AFB2); or includes a sequence having 50% sequence identity to the sequence set forth in SEQ ID NO: 1.

29. The method of embodiment 27, wherein the auxin response factor has one or more of the following characteristics: includes a DNA-binding domain (DBD) and a Phox/Bem1p (PB1) domain; binds the auxin-responsive protein and an auxin response element; is auxin response factor 19 (ARF19); or includes a sequence having 50% identity to the sequence set forth in SEQ ID NO: 2.

30. The method of embodiment 29, wherein the auxin response element (when present) includes a sequence upstream of the reporter including a TGTCxx sequence motif.

31. The method of embodiment 30, wherein the TGTCxx sequence motif includes the TGTCTC sequence or TGTCGG sequence.

32. The method of embodiment 28, wherein the reporter includes a fluorescent reporter or a luminescent reporter.

33. The method of embodiment 32, wherein the fluorescent reporter is a Venus fluorescent reporter.

34. The method of embodiment 23, wherein the plasmid further includes: an auxin receptor, an auxin response factor, and a reporter, such that the genetically modified cell expresses the auxin receptor, the auxin response factor, and the reporter.

35. The method of embodiment 34, wherein the auxin receptor has one or more of the following characteristics: includes an F-box domain and a leucine-rich repeat (LRR) domain; binds auxin (indole-3-acetic acid); is auxin-signaling F-box 2 (AFB2); or includes a sequence having 50% sequence identity to the sequence set forth in SEQ ID NO: 1.

36. The method of embodiment 34, wherein the auxin response factor has one or more of the following characteristics: includes a DNA-binding domain (DBD) and a Phox/Bem1p (PB1) domain; binds the auxin-responsive protein and an auxin response element; is auxin response factor 19 (ARF 9); or includes a sequence having 50% identity to the sequence set forth in SEQ ID NO: 2.

37. The method of embodiment 36, wherein the auxin response element (when present) includes a sequence upstream of the reporter including a TGTCxx sequence motif.

38. The method of embodiment 37, wherein the TGTCxx sequence motif includes the TGTCTC sequence or TGTCGG sequence.

39. The method of embodiment 35, wherein the reporter includes a fluorescent reporter or a luminescent reporter.

40. The method of embodiment 39, wherein the fluorescent reporter is a Venus fluorescent reporter.

41. The method of embodiment 23, wherein the cell is a yeast cell, and transforming includes at least one of: suspending the yeast cell in lithium acetate solution and contacting the yeast cell with the plasmid; or contacting the yeast cell with the plasmid and heating the yeast cell.

42. The method of embodiment 23, further including selecting transformed reporter cells after transforming the cell.

43. The method of embodiment 42, wherein selecting includes positive selection or negative selection.

44. The method of embodiment 23, further including screening a bioactive molecule, wherein the screening includes contacting the transformed cell with the bioactive molecule and determining repression activity.

45. The method of embodiment 44, wherein the bioactive molecule includes one or more of: a small molecule; a peptide or protein; a natural product; a synthetic bioactive compound; an anti-cancer drug; or the anti-cancer drug BC 2059 (Tegavivint).

46. The method of embodiment 44, wherein determining repression activity includes performing one or more of: a transcription-based assay; flow cytometry; a Western blot assay, microscopy, a fluorescence assay, or a luminescence assay.

47. The method of embodiment 23, wherein the cell is a metazoan cell, a fungal cell, an algal cell, or a plant cell.

48. The method of embodiment 47, wherein the metazoan cell is a fish cell, an amphibian cell, a reptile cell, a mammalian cell, a bird cell, or an insect cell.

49. The method of embodiment 47, wherein the fungal cell is a yeast cell.

50. The method of embodiment 49, wherein the yeast cell is a Saccharomyces cerevisiae yeast cell.

51. The method of embodiment 23, wherein the cell is a plant cell or a plant protoplast.

52. The method of embodiment 23, wherein the cell has been transiently or stably transformed with the plasmid to produce the genetically modified cell.

53. The method of embodiment 23, wherein the yeast cell is from a haploid strain or diploid strain.

54. The method of embodiment 23, wherein the LisH sequence of interest includes a library of LisH variants; the plasmid is a plasmid library of the library of LisH variants; and a plurality of cells are transformed with the plasmid library.

55. The method of any one of embodiments 23-54, wherein the LisH Domain includes the sequence of any one of SEQ ID NOs: 8-111 or 113-130.

56. The method of any one of embodiments 23-54, wherein the LisH Domain includes an alpha-helix including an amino acid sequence XX[I/V/L]XX[Y/I/V/L][I/V/L]XXX[L]XX, wherein “X” can be any amino acid.

57. A method of using the genetically modified cell of any one of embodiments 1-22 for genetic mutation testing.

58. The method of embodiment 57, wherein the genetic mutation testing includes cancer mutation (e.g., oncogene) testing.

59. A method of using the genetically modified cell of any one of embodiments 1-22 for developmental mutation testing.

60. A method of using the genetically modified cell of any one of embodiments 1-22 for agricultural mutation testing.

61. A method of using the genetically modified cell of any one of embodiments 1-22 for small molecule testing.

### (XII) EXPERIMENTAL EXAMPLES

### Example 1. A Single Helix Repression Domain is Functional Across Eukaryotes

Transcriptional repression by corepressors is essential to life, development, and response to environment. The mechanisms by which corepressors regulate gene expression across eukaryotes are unresolved. The plant corepressor TOPLESS (TPL) is one such essential corepressor that contains a Lis1 Homology domain (LisH). While the LisH domain has been broadly characterized as a homo-dimerization domain, the TPL LisH is sufficient to enact transcriptional repression and it has previously shown that the first 18 amino acid long alpha-helical region (H1) is a minimal repression domain. The LisH domain is also found in proteins with functions in ubiquitination and cytoskeletal dynamics that do not have described repressive functions. However, Lis1, the founding member of the family, has recently been described to have repressor activity (Leydon et al., eLife 10, e66739, 2021). Therefore, it was hypothesized that LisH H1 domains across eukaryotic proteins may have a conserved transcriptional repressor activity. As described below, the conservation of LisH H1 function was interrogated using an Auxin Response Circuit in Saccharomyces cerevisiae (ARC^{Sc}) and key residues within H1 that contribute to function were identified.

This function was tested across eukaryotic proteins using libraries of extant LisH H1 sequences, and repression was found to be highly conserved throughout. The role of documented somatic cancer mutations was also tested in the human LisH-containing proteins TBL1 and DCAF1 for effects on repressive function; the results demonstrate that the assay provide herein enables quantification of observed disease-related variants. The findings suggest that H1 has ancestral repressive capacity. This study identifies short orthogonal repressor sequences for synthetic biology applications applicable across eukaryotes.

As described in this Example, yeast was used to interrogate the origins of the LisH domain's helix 1 (H1) repressive function across eukaryotes. Libraries of H1 sequences were used in the AtARCSc to test the function of TPL-H1 residues that control robust transcriptional repression. A library of H1 sequences from diverse proteins across eukaryotes was then used to test the extent of H1 repressive function across both species and proteins of different annotated functions. Yet another set of libraries allowed for quantification of the effect of somatic cancer mutations on TBL1 and DCAF1 stability and function, helping to connect H1 repressive function to oncogenesis. Finally, H1 sequences were tested for viability as a synthetic protein tag for tunable transcriptional repression in a plant system. These findings uncover the ancestral transcriptional repression ability of the LisH domain, showcase how this system can be used to understand disease states, and can be applied as a transcriptional repressor in synthetic biology.

At least some of the results described herein were published in Leydon et al. (PNAS 119(41):e2206986119, Oct. 3, 2022; doi.org/10.1073/pnas.2206986119) and the accompanying supplemental materials. In some instances, the figures from that publication (which may also be present in priority Application No. 63/338,637, filed May 5, 2022) are referred to herein; in such instances, the reference is to each “Figure”, whereas references to figures included in the subject application are designated by FIG. numbers.

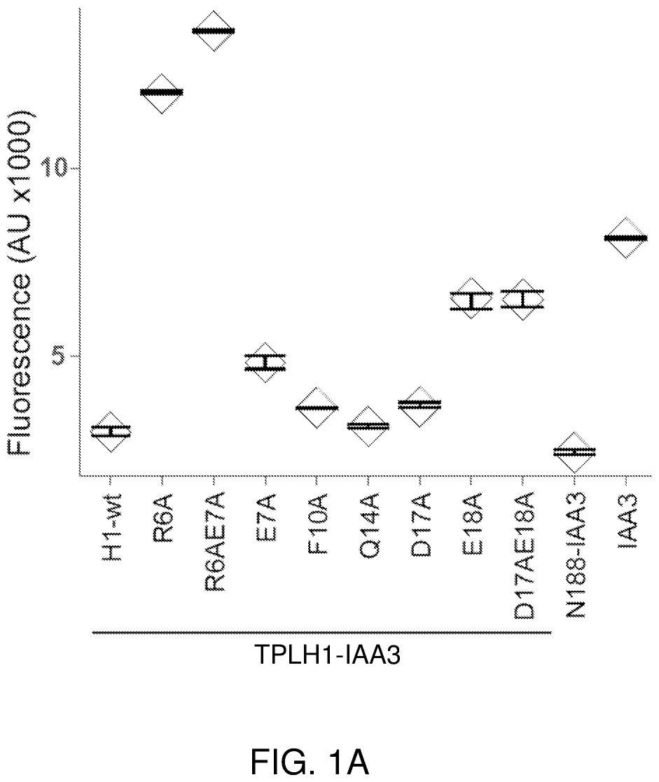

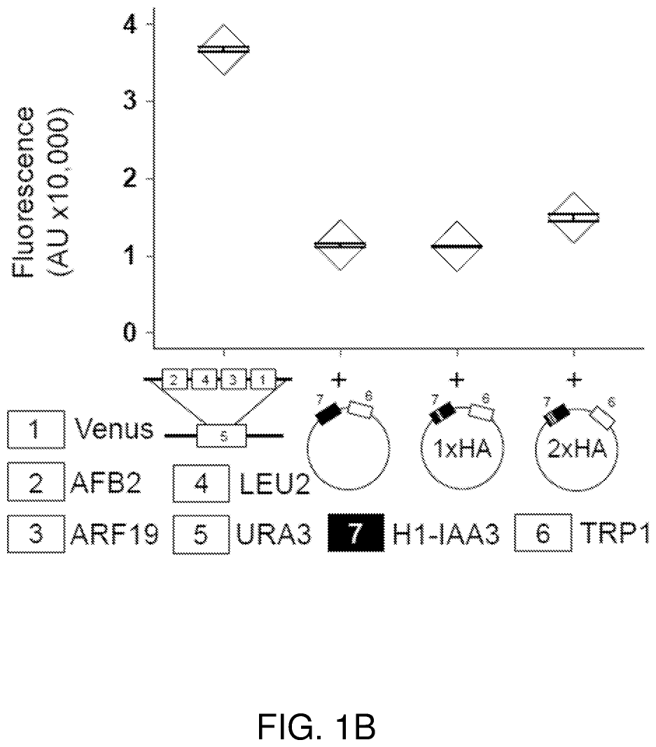

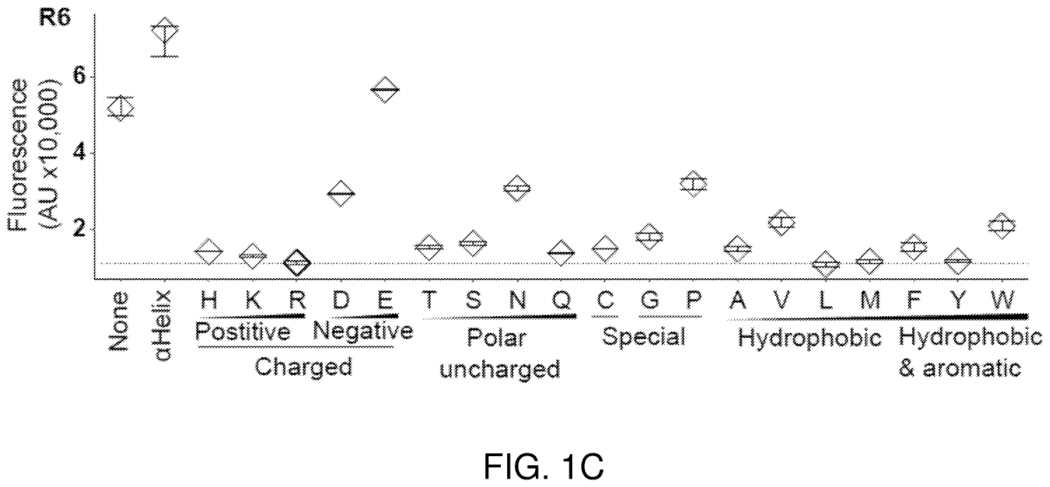

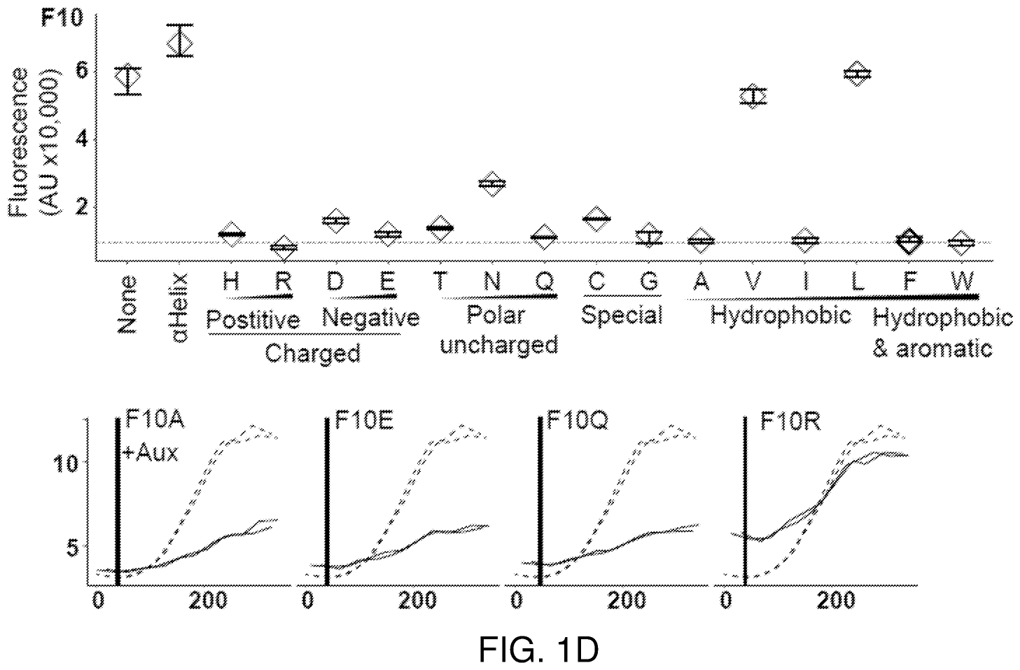

Results. The TPL LisH domain is a short transcriptional repression domain. To understand how TPL represses transcription, the small modular LisH domain was focused on which was previously demonstrated to be sufficient to repress transcription in the Arabidopsis thaliana Auxin Response Circuit in Saccharomyces cerevisiae (AtARCSc) (Leydon et al., eLife 10, e66739, 2021; Pierre-Jerome et al., Proc. Natl. Acad. Sci. U.S.A 111, 9407-9412, 2014). The AtARCSc allows for the measurement of auxin-relievable TPL repressive function when directly fused to auxin co-receptor IAA3. It was identified that a construct carrying only the first 18 amino acids of Helix 1 of the LisH domain fused to IAA3 (H1-IAA3) was sufficient to confer repression (H1, FIG. 1A of Leydon et al., 2022 (which shows the sequence and structure of Helix 1 (H1) (PDB: 5NQS). The LisH domain is dark grey in Helix 1 and Helix 2, and amino acids chosen for mutation are in light grey and annotated. In the sequence, amino acids chosen for mutation are underlined), FIG. 1A), and that this H1-IAA3 fusion protein construct behaves identically to TPLN100-IAA13 (Pierre-Jerome et al., Proc. Natl. Acad. Sci. U.S.A 111, 9407-9412, 2014). To pinpoint which residues of H1 could coordinate repression through interaction with other transcriptional regulators, likely solution-facing amino acids were identified. It was hypothesized that these amino acids were not involved in stabilizing the hydrophobic interactions between intra-TPL helical domains and could interact with partner proteins (Martin-Arevalillo et al., Proc. Natl. Acad. Sci. USA, 2017, doi.org/10.1073/pnas.1703054114; Ke et al., Sci. Adv. 1, e1500107, 2015). Six amino acids in H1 were each mutated to alanine (FIG. 1A of Leydon et al., 2022, pink residues) in the context of H1-IAA3. Repression activity was assessed in the absence of auxin. The amino acids on either end of the helix (R6 and E18) were required for repression (FIG. 1A) as high reporter expression is observed. A mutation of E7, the immediate neighbor of R6, slightly increased reporter expression (FIG. 1A) and lowered the final activation level after auxin addition when compared with wild type H1 suggesting it only plays a supporting role in repressive function (FIG. 1C of Leydon et al., 2022, which shows a time course flow cytometry of selected mutations of Helix 1 following auxin addition. Auxin (IAA-10 μM) was added at the indicated time (gray bar, +Aux)). The R6A, E7A double mutation behaved similarly to the R6A single mutation. Likewise, the D17A, E18A double mutation did not enhance the effect of E18A alone. Q14A and D17A were indistinguishable from wild type H1 (FIG. 1C of Leydon et al., 2022). In contrast to the other mutations which reduced H1 repression activity or had no effect, F10A strengthened the durability of repression H1, converting it into an auxin-insensitive repression domain (FIG. 1C of Leydon et al., 2022). In the context of the full-length N-terminus of TPL (i.e. TPLN188), F10 sits underneath the linker that connects Helix 8 to Helix 9, interacting with inward-facing hydrophobic cluster formed by F10 and F163, F33, F34 and L165. It is likely that in any truncations where the linker is removed (i.e., TPLN100), F10 negatively affects repressor activity, possibly by decreasing binding affinity to putative interaction partners or the stability of protein complexes.

In order to rapidly test an expanded repertoire of mutations in LisH H1 sequences, a new auxin response circuit (ARC) was designed that includes an epitope tag to allow quantification of repressor protein levels (FIG. 1D of Leydon et al., 2022, which shows a schematic of HA epitope placement and western blots of tagged constructs). All constant parts were integrated at the URA3 locus, and a H1-1xHA-IAA3 fusion protein was selected upon which to perform further mutational analysis (FIG. 1B; Figures S1A-S1F of Leydon et al., 2022). As illustrated in Figures S1A-S1F, the single plasmid auxin response circuit (ARC) uses a hybrid integrated/unintegrated yeast auxin response circuit. Fluorescence flow cytometry on strains containing the ARC split into two plasmids with (4412) or without (4455) an H1 repressor. In circuits with an unintegrated reporter, repression was observed, yet there was a wide peak width, limiting the resolution between the repressed and de-repressed response states. Integration of all components except the repressor led to tighter peak width distributions and increased the resolution of the repressed state when tested by fluorescence flow cytometry. Figure S1C provides a schematic of engineered versions of the H1-IAA3 repressor with single (1×) or double (2×) HA epitope tags, and Western blots with antibodies against HA and PGK1. A single HA epitope was sufficient for detection. Figure S1 D illustrates fluorescence flow cytometry of epitope tagged H1-IAA constructs. A summary of fluorescence flow cytometry is show in Figure S1E. For all flow cytometry each panel represents two independent time course flow cytometry experiments of the TPL helices indicated, all fused to IAA3, every plot represents the average fluorescence of at least 10,000 individually measured yeast cells (a.u. —arbitrary units).

To better understand the properties of residues in TPL-H1 that contribute to its function amino acid swapping experiments were designed at residues R6 and F10 that were sensitive to alanine mutations (FIG. 1A, and FIG. 1C of Leydon et al., 2022). In the case of R6, broad tolerance of amino acid swap was observed, with the exceptions of charge inversion (R6D and R6E), and an unsurprising reduction of repression in R6P, which is likely to interfere with its alpha-helical structure. While it is surprising that R6A has only a mild decrease in repression in this experiment, it is more than likely due to the new stoichiometry of plasmid based TPL-H1 (higher expression) to the integrated AtARCSc components, meaning this assay is slightly less sensitive to loss of function than integrating all components. Several amino acid swaps had a negative effect on protein accumulation (R6E, R6N, and R6V), resulting in loss of repression that is likely due to this lower abundance. In all experiments these mutations were compared to a well characterized alpha-helical linker sequence (α-helix-HA-IAA3) as a control, as well as IAA3 (None) alone (FIGS. 1C, 1D).

In the F10A mutation a loss of auxin sensitivity was observed (FIG. 1C of Leydon et al, 2022, H1-F10A), suggesting that the wild-type F10 residue was either reducing initial protein abundance or auxin-induced degradation when exposed in the H1 truncation. In order to test the effect on changing physicochemical amino acid class, the effect of a selection of amino acid swaps at this position were tested. It was observed that L, V and N amino acids negatively affected protein levels, which also reduced repression (FIG. 1D). Several amino acid swaps appeared to have equivalent repression to wild type: R, Q, A, I, and W.

The LisH H1 sequences are a powerful synthetic biology tool that will allow for the creating of transcriptional repressors in a number of eukaryotic systems through a short, modular and organism-orthogonal tag. Because the TPL-H1 is tolerant to positional changes it also becomes possible to further derive completely orthogonal sequences based on the Arabidopsis TPL for plants, allowing for tuning repressive strength with unique encoding sequences. Each TF-repressor fusion requires protein optimization as other attempts to use the full-length TPL have met with varied success (Gander et al., Nat. Commun. 8, 15459, 2017; Khakhar et al., eLife 7, 2018; Brophy et al., Science 2022.02.02.478917, 2022), likely due to repressor domain positioning or linker length and flexibility. One attractive approach is to recruit H1 sequences via the SunTag approach, which allows multiple peptide recruitment in a sterically-flexible manner (Pan et al., Nat. Plants 7, 942-953, 2021), where they might be less dependent on exact domain arrangement.

Defining the LisH H1 sequence. Although the transcriptional functions of LisH H1 domains in proteins other than TPL have not been directly tested for repressor activity, many LisH-containing proteins including LUG, HOS15, TBL1, and SIF2 are important transcriptional regulators (Wong et al., Am. J. Clin. Exp. Urol. 2, 169-187, 2014; Conner et al., Proc. Natl. Acad. Sci. U.S.A 97, 12902-12907, 2000; You et al., Plant Cell, tpc.00115.2019, 2019; Mayer et al., Plant Physiol. 180, 342-355, 2019; Cockell et al., Curr. Biol. CB 8, 787-790, 1998). Indeed, many LisH-containing proteins are unlikely to be predicted as transcriptional regulators, since they are primarily cytoplasmic, or have well-studied primary functions in ubiquitination or cytoskeletal dynamics. However, Lis1, containing the founder LisH domain, has been discovered to moonlight as a transcriptional regulator (Keidar et al., Front. Cell. Neurosci. 13, 2019), suggesting other LisH containing proteins may retain the capacity to regulate transcription. To understand the roles of the LisH H1 in transcriptional regulation and determine how widespread this function may be requires understanding the diversity of LisH H1 sequences and functionally characterizing their repressive functions in a single comparative experiment.

Maximum Likelihood (ML) reconstruction were performed of LisH H1 sequences sampled from diverse LisH-containing proteins across eukaryotes to better understand the diversity of H1 sequences and the proteins that carry them (FIG. 2A). This is also illustrated in Figure S2 of Leydon et al., 2022, which provides the extended phylogeny for FIGS. 2A-2C. The evolutionary history of LisH-H1 sequences was inferred by using the Maximum Likelihood method and Le Gascuel, 2008 model (Le & Gascuel, Mol. Biol. Evol. 25, 1307-1320, 2008). The tree with the highest log likelihood (−2709.60) is shown. The percentage of trees in which the associated taxa clustered together is shown next to the branches. Initial tree(s) for the heuristic search were obtained automatically by applying Neighbor-Join and BioNJ algorithms to a matrix of pairwise distances estimated using the JTT model, and then selecting the topology with superior log likelihood value. A discrete Gamma distribution was used to model evolutionary rate differences among sites (5 categories (+G, parameter=6.1034)). The tree is drawn to scale, with branch lengths measured in the number of substitutions per site. This analysis involved 143 amino acid sequences. There was a total of 14 positions in the final dataset. Evolutionary analyses were conducted in MEGA X (Felsenstein, J. Evol. Int. J. Org. Evol. 39, 783-791, 1985). The percentage of replicate trees in which the associated taxa clustered together in the bootstrap test (1000 replicates) are shown next to the branches (Kumar et al., Mol. Biol. Evol. 35, 1547-1549, 2018).

While H1 sequence length is too short (only 18 amino acids) to produce optimal bootstrap values (Roch & Sly, Probab. Theory Relat. Fields 169, 3-62, 2017), the reconstruction allows for making associations regarding sequence similarity. Sequences cluster into five main clades which are defined by nodes marked I-V. Some of these H1 sequences were identical across orthologous genes, and therefore represent many LisH sequences found in genes other than the gene name assigned to the sequence in our cladogram (Tables 1-3). Representative protein's functions and cellular localizations were annotated to create hypotheses about their H1's roles in repression (Table 2). H1 alignments allow for the finding of residues of interest across genes and clades (FIG. 2B). His were characterized as having conserved residues L8, N9, L11, 112, L16, and Y15 (FIG. 2B), which determine H1 identity, and they include the inward facing LisH dimerization interface. It was hypothesized that solvent-facing residues of high diversity that differentiated these clades would likely be determinants of repressive function.

LisH repressive function appears to be widely conserved and ancestral. The synthetic ARC in yeast allows direct comparison of repressive function across distantly related H1 sequences. To test diverse sequences, a representative plasmid library (see Tables 4 & 5) was created using the H1 sequences in the ML reconstruction (FIG. 2A) to experimentally test the repressive function of these sequences and introduced them to yeast. Generally, it is difficult to detect repression in proteins that accumulate at relatively low levels, however high accumulation does not seem to correlate with increased repression (FIG. 2B; Figure S3 of Leydon et al., 2022). Figure S3 provides repression assay data visualizations. Cytometry data is as represented in FIGS. 2A-2C, this time ordered by (top panel of Figure S3) how much repression was detected (with proteins highly over or under expressed removed), and (bottom panel of Figure S3) how much protein accumulated. Protein accumulation was tested by western blot and normalized to yeast PGK1. Protein level was normalized to wild type TPL H1 (dotted line) and each data point is color coded on a log 2 scale, with blue and red indicating the lowest and highest expressions, respectively. Each panel represents two independent time course flow cytometry experiments of the TPL helices indicated, all fused to IAA3. For all cytometry, every point represents the average fluorescence of at least 10,000 individually measured yeast cells (a.u. —arbitrary units).

It was determined that H1s that belong to genes characterized as nuclear transcriptional repressors, such as ScSIF2, AtHOS15, and AtLUG robustly repressed reporter activity similar to AtTPL (FIG. 2C). However, many of the strongest repressors were found in genes across the tree without previously characterized roles in transcriptional repression like HsSMU1, SpADN2, and NcSSH4. Clade I H1s have high sequence diversity (FIGS. 3A-3F), and due to their relation to TPL, it was hypothesized these H1s are the most likely to also have a repressive function, which is consistent with the experimental results. Clade I H1 sequences from proteins involved in protein ubiquitination SmRANB9, Hs & AtDCAF1, and LuDDB1 have repressive function, as does the splicing factor HsSMU1 (Ulrich et al., Struct. Lond. Engl. 1993 24, 762-773, 2016). Repression by SpHIF2 H1 was not detected, however this is likely due to very poor protein stability.

It was observed that H1 sequences from cade II were characterized by a slightly lower sequence variability than cade I and a high incidence of R14 and E18 residues (FIGS. 2B, 3A-3F). H1s from the repressor proteins ScSIF2 (Cerna & Wilson, J. Mol. Biol. 351, 923-935, 2005) and AtHOS15 (Mayer et al., Plant Physiol. 180, 342-355, 2019) repress strongly, while H1s closer in sequence to HsTBL1X repress less strongly. Interestingly, TBL1 has been described as an exchange factor, where during repression recruits the repressive SMRT/NCoR complex and upon stimulus facilitates the recruitment of transcriptional activators to the locus (Perissi et al., Cell 116, 511-526, 2004). It is interesting to speculate that TBL1 H1s have been evolutionarily selected on to have lower intrinsic repressive activity to permit better exchange activity.

Clade III H1 sequences are defined by their similarity to GID8, a member of the yeast multiprotein E3 ligase GID complex (Sherpa et al., Mol. Cell 81, 2445-2459.e13, 2021), also known as the CTLH (carboxy-terminal to LisH) complex in mammals (Maitland et al., Sci. Rep. 9, 9864, 2019). Clade III sequences are well conserved, with a high incidence of M13, and N14 residues (FIGS. 2B, 3A-3F), which are on the solvent facing side of H1 suggesting that these residues might reduce H1s repressive functions by virtue of lowering affinity with repressive partner proteins. Most cade Ill H1s are expressed at levels higher than AtTPL, and sequences with both E6 and D7 show the lowest capacity to repress, suggesting that these H1 domains from the GID/CTLH complex do not retain transcriptional repression capacity.

Clade IV H1s include the nuclear localized transcriptional activators SpADN2, SpADN3 and ScMSS11, as well as the plant corepressors LEUNIG (LUG) and its homolog (LUH, Table 2). They have a high incidence of Y11, Y13, and K18 residues and many of these sequences negatively affected protein abundance (FIGS. 2B, 3A-3F). Surprisingly, SpADN2 and ScMSS11 H1s were repressive. Of the LUG and LUH sequences, only AtLUG was comparable to AtTPL, however many of the LUH H1 sequences negatively affected protein stability. Similar to TBL1, LUG and LUH have been described to be important for both repression and activation (Zhang et al., New Phytol. 223, 2024-2038, 2019; Gonzalez et al., Mol. Cell. Biol. 27, 5306-5315, 2007), suggesting that it's H1 may have lost strong repressor activity through evolution. The L13Y variation may contribute to this loss, since L13 is a highly conserved residue elsewhere.

Finally, clade V includes highly diverse sequences belonging to genes coding for both nuclear and non-nuclear localized proteins with no annotated transcriptional functions (see Leydon et al., 2022). The HsLIS1 H1 sequence is both well expressed and repressive, pointing to a possible role for this sequence in mediating repression in its repressive role with MeCP2 (Keidar et al., Front. Cell. Neurosci. 13, 2019). Interestingly, unlike GID8, His from other GID/CTLH complex members such as SmMAEA and HsRANB9 retain repressive ability, which suggests these might have roles in regulation of gene expression.

To further support these findings, His were ancestrally reconstructed and tested at nodes of interest across a ML tree (see Figures S5A-S5D of Leydon et al., 2022; Figures. 8A-8E of U.S. Provisional Application No. 63/338,637; as well as FIGS. 2A and 2C). Ancestral Sequence Reconstruction is provided in Figures S5A-S5D, along with a simplified cladogram of a ML tree is shown highlighting the nodes where ancestral sequences were inferred, marked by dashed lines. Extant H1 sequences are used to contextualize branches. Notable genes within each branch of the reduced tree are labeled, and predicted ancestral sequences are named after the major functions of genes associated with downstream sequences. Sequences ordered and tested (black) and sequences not included in this experiment (gray). H1 sequences tested experimentally in this experiment (black) and in others (gray) were aligned and residues colored by their physicochemical class. The consensus sequence for H1 is aligned alongside the relative conservation rate of different residues along the helix. The relative repressive function of different H1s was measured using fluorescence flow cytometry. Dashed lines mark the fluorescence of the TPLH1 control, and the noH1 control. Protein levels were normalized to TPLH1, with those accumulating at lower levels marked in blue, and those at higher levels marked in red. The evolutionary history of LisH-H1 sequences was inferred by using the Maximum Likelihood method and Le_Gascuel_2008 model (PMID: 18367465). The tree with the highest log likelihood (−2709.60) is shown. The percentage of trees in which the associated taxa clustered together is shown next to the branches. Initial tree(s) for the heuristic search were obtained automatically by applying Neighbor-Join and BioNJ algorithms to a matrix of pairwise distances estimated using the JTT model, and then selecting the topology with superior log likelihood value. A discrete Gamma distribution was used to model evolutionary rate differences among sites (5 categories (+G, parameter=6.1034)). The tree is drawn to scale, with branch lengths measured in the number of substitutions per site. This analysis involved 143 amino acid sequences. There was a total of 14 positions in the final dataset. Evolutionary analyses were conducted in MEGA X (Kumar et al., Mol. Biol. Evol. 35, 1547-1549, 2018). This data was not bootstrapped. The final tree was used to infer ancestral sequences.

All reconstructed sequences are repressive with the exception of the clade IV sequence. It contains the L11Y variation which was previously hypothesized to play a role in decreased H1 function in the activator clade. Although some members of this clade can elicit repression, a trend towards decreased repression in this clade is observed, and many genes in this clade are annotated in the literature as being involved in transcriptional activation. Sequences from clade I and III both have an R6E substitution, which in AtTPL was associated with decreased repressive function, likely driven by the switch in polarity. Only a few conserved residues in the H1 helix are responsible for its repressive function, allowing for lots of sequence plasticity, which can tune repression strength.

Repressive function is found in H1s across all clades measured as well as basal H1s such as CaFLO8 and SpRAN suggesting that this function is a widespread and highly conserved characteristic of the LisH H1. Furthermore, residues outside of the conserved core LisH H1 motif (including hydrophobic amino acids L8, 11, 16 and 112) serve mainly to tune the repressive function. This indicates that the most important determinant of repression is the multimerization interface and suggests that most LisH H1 sequences should retain this activity when localized to chromatin. In these assays only the H1 sequence is tested, which cannot homo-dimerize on its own (Leydon et al., eLife 10, e66739, 2021).

LisH domains are important for human disease. The human oncogene HsTBL1X is a transcriptional regulator and exchange factor involved in transcriptional repression and activation (Perissi et al., Nat. Rev. Genet. 11, 109-123, 2010; Choi et al., Mol. Endocrinol. 22, 1093-1104, 2008; Guenther et al., Genes Dev. 14, 1048-1057, 2000; Perissi et al., Cell 116, 511-526, 2004), and is implicated in the progression of multiple cancers (Wong et al., Am. J. Clin. Exp. Urol. 2, 169-187, 2014; Choi et al., Mol. Cell 43, 203-216, 2011; Li & Wang, Nat. Cell Biol. 10, 160-169, 2008). The first-in-class anti-cancer compound Tegavivint targets the specific interaction of TBL1 and Wnt at the TBL1 N-terminal LisH domain and is the subject of several ongoing clinical trials (Soldi et al., J. Pharmacol. Exp. Ther. 378, 77-86, 2021; Children's Oncology Group, NSC #826393 (clinicaltrials.gov, 2022) (Mar. 17, 2022); Nomura et al., JNCI J. Natl. Cancer Inst. 111, 1216-1227, 2019). All human TBL1 genes (TBL1X, TBL1XR1, TBL1Y) contain a LisH domain, and it was identified that the N-terminal region of TBL1X (residues 1-76; FIG. 3A of Leydon et al., 2022) has the ability to repress in the synthetic circuit when fused to IAA3, but not as well as TPL (see FIG. 3B of Leydon et al., 2022, dashed lines). TBL1's H1 exhibited a similar repression ability to the TBL1 N-terminus and was de-repressible in the AtARC with the addition of auxin, similar to the TPL H1 (see FIG. 3B of Leydon et al., 2022, solid lines).

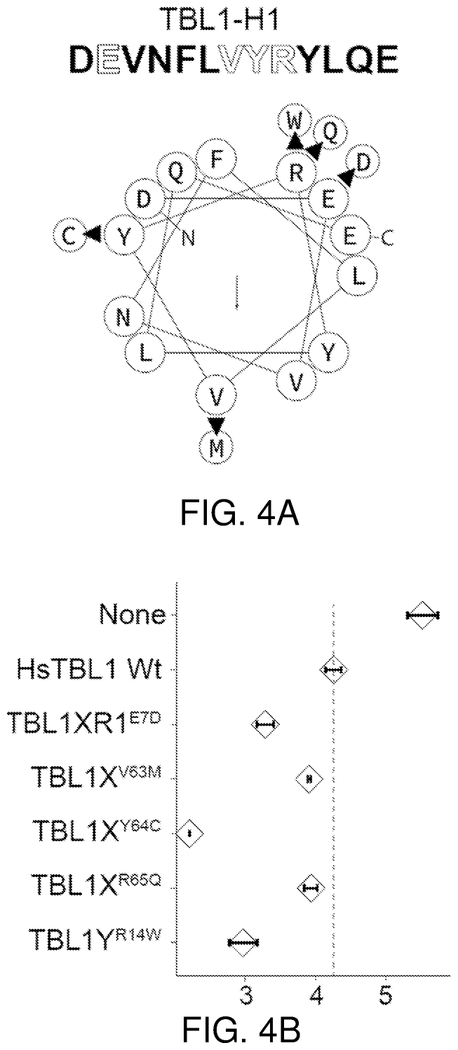

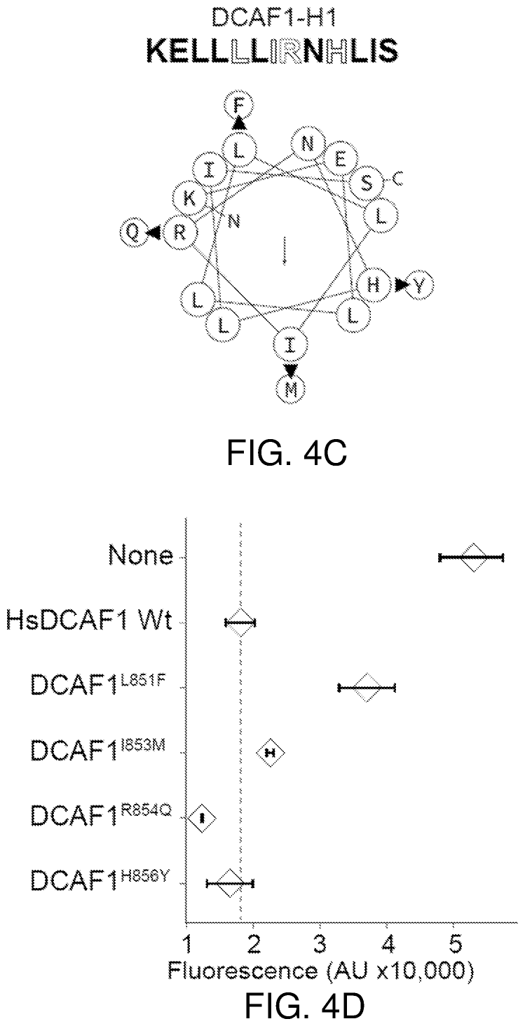

The COSMIC database was queried as a testcase for using the ARC for functional analysis of cancer-associated variants (Tate et al., Nucleic Acids Res. 47, D941-D947, 2019), and identified five non-synonymous mutations occurring in the HsTBL1 H1 (pooled mutations from TBL1X, TBL1XR1, TBL1Y, FIG. 4A). It was hypothesized that mutations occurring in this helix play a role in disease, and these likely play a role by altering the repressive function of H1.

In order to test this, these mutated H1 sequences were recreated, and their repressive function was tested with ARC and cytometry. All annotated mutations were found to increase HsTBL1 repressive function. HsTBL1X Y64C was the strongest repressor, and also demonstrated the highest accumulation, suggesting this mutation increases HsTBL1 function by increasing protein stability. It is interesting to note that both R65Q and R14W both demonstrate reductions in protein abundance yet higher repression rates, identifying these as significantly better repressors. Cancer-associated mutations in HsTBL1 are associated with increased repression, suggesting that increased HsTBL1 repressive function must be factored in as a potential driver of cancer development or progression. See also Example 3.

A number of LisH containing proteins in the phylogeny are components of E3 ubiquitin ligase complexes, one of which is a substrate receptor for Cullin RING ligase 4 (CRL4) and is named DDB1 (DNA damage-binding protein 1) and CUL4-associated factor 1 (DCAF1; Schabla et al., J. Mol. Cell Biol. 11, 725-735, 2019). DCAF1 has been extensively studied for its involvement in regulating many cell processes, including its role in cancer (Schabla et al., J. Mol. Cell Biol. 11, 725-735, 2019) as well as its subversion by HIV viral accessory proteins (Zhang et al., Gene 263, 131-140, 2001). Following the same methodology as with TBL1, the existing somatic mutations in HsDCAF1 were mined to find and quantify the effects of existing human variation on transcriptional repression and tested four non-synonymous mutations in the HsDCAF1 H1 domain (FIG. 4C). Unlike HsTBL1X, Wt HsDCAF1 H1 has a relatively strong repressive function (FIG. 4D). This repressive function and protein accumulation are not significantly affected by mutation H856Y. 1853M and R854Q slightly decrease and increase repressive function, respectively, as well as slightly increasing protein accumulation. However, L851F dramatically reduces H1 stability and repressive function.

The DCAF1 LisH has been implicated in both dimerization (Ahn et al., Biochemistry 50, 1359-1367, 2011) and transcriptional repression, where it has been demonstrated to inhibit p53's transcriptional activity through binding of hypoacetylated Histone 3 tails (Kim et al., Mol. Cell. Biol. 32, 783-796, 2012; Wang et al., Nature 538, 118-122, 2016). Likewise, TBL1 has been demonstrated to bind to hypoacetylated tails of histone H4 and H2B, and that this contact is required in addition to the specific transcription factor interaction that recruits the SMRT/NCoR complex (Yoon et al., Mol. Cell. Biol. 25, 324-335, 2005). It will be critical to identify whether the conserved mechanism of H1 activity is based upon this same interaction. Further experiments will be required that leverage the H1 in the ARC to rapidly connect protein sequences—such as the cancer variants trialed here—to function. These assays could be relevant for the design and use of interventions based on specific cancer subtypes.

The H1 can act as a synthetic repressor domain in planta. Many His from distantly related species seem to work in inducing transcriptional repression in yeast, therefore testing whether H1 sequences could be used as short repression tags in a model plant was desired. These short repressors could theoretically be used as tags to make proteins of interest that behave as repressors, or create hormone-responsive de-repressible systems which may help to activate gene expression based on environmental cues, cell identity, or exogenous chemical applications (Khakhar et al., eLife 7, 2018; Leydon et al., Annu. Rev. Plant Biol. 71, 767-788, 2020). In order to determine the transferability of the results in yeast to synthetic biology applications in plants, the H1-HA-IAA3 cassette was transferred into a plant compatible vector. In this process, the IAA3 EAR motif was ablated to eliminate the possibility of recruiting endogenous TPL/TPR family repressors.

Transient transformation assays were performed in Nicotiana benthamiana, to test the ability of H1 to repress the synthetic auxin reporter DR5-Venus. Reporter activation was measured in four separate leaf injections (biological replicates) in two days of injection (boxplots illustrated in FIG. 5C are pooled data from one day, with two replicates divided on right and left panels). Each leaf was excised at 8 locations and measured for Venus fluorescence using a plate scanner. pDR5:Venus: the synthetic DR5 auxin promoter (Ulmasov et al., Plant Cell 9, 1963-1971, 1997) driving Venus; ARF19: p35S:AtARF19-1×FLAG; Each H1 sequence is identical to the H1-HA-IAA3 construct used in FIGS. 2A-2C except the LxLxL EAR sequence has been mutated to AxAxA as to not recruit endogenous TPL/TPR proteins in N. benthamiana.

H1s were able to selectively repress reporter expression in transient transformations in planta. Similar results were observed in AtTPL sequence variants between yeast and plants, especially for the F10A mutation, which shows improvements in repression in tobacco compared to wild type, consistent with its auxin-insensitivity in the AtARCSc (see FIG. 1C in Leydon et al., 2022). Sixteen of the best repressor sequences were tested from the yeast assay in tobacco and detected repression with nearly all tested sequences (FIG. 5C). Certain H1s appear to be less (i.e. PfGID8) or not functional (i.e. DmGID8) in tobacco, and suggests that several H1s should be tested in an organism as repressor domains to evaluate the ideal performance. However, the fact that LisH Helix 1 domains from different species work in yeast and plants reinforces that this mechanism of repression is highly conserved across eukaryotes.

Conclusions. This example points to an ancestral repression ability to the LisH Helix 1 domain and raises the question of whether many more LisH containing proteins are performing a role in transcriptional regulation. What roles are due to possible transcriptional activities can now be dissected, including in cases where LisH containing proteins are already involved in many biological processes. The ARC^{Sc} is a powerful system, allowing for the study of transcriptional repression that is evolutionary, related to disease, related to structure, sequence identity, and more. In the cases of mutations playing roles in oncogenesis, the synthetic platform becomes an orthogonal testing ground to determine whether these mutations have specific effects on protein abundance and transcriptional activity. Furthermore, because of the ease of high-throughput analysis, the ARC is an ideal system to perform small molecule screening for evaluation of current therapeutics as well as to specifically design and synthesize libraries focused on a particular target or mechanism of action.

Methods. Alignments and Phylogenetic Reconstruction. LisH domains were identified using UniProt (uniprot.org/), Pfam (pfam.xfam.org/family/PF08513.7) and SMART (smart.embl-heidelberg.de/) databases. LisH Helix 1 domains were aligned using Clustal Omega. Tree sequences were selected from the PFAM LisH clade PF08513 by performing an alignment of the representative proteome dataset with a 15% cutoff value (1235 sequences). The evolutionary history was inferred by using the Maximum Likelihood method and Le_Gascuel_2008 model (Le & Gascuel, Mol. Biol. Evol. 25, 1307-1320, 2008). The tree with the highest log likelihood (−2711.40) was used. Initial tree(s) for the heuristic search were obtained automatically by applying Neighbor-Join and BioNJ algorithms to a matrix of pairwise distances estimated using the JTT model, and then selecting the topology with superior log likelihood value. A discrete Gamma distribution was used to model evolutionary rate differences among sites (5 categories (+G, parameter=6.6850)). The percentage of replicate trees in which the associated taxa clustered together were calculated via bootstrap using 1000 replicates (Felsenstein, Evol. Int. J. Org. Evol. 39, 783-791, 1985). This analysis involved 143 amino acid sequences. The cladogram was derived from this tree, only showing relationships among the 63 experimentally analyzed sequences. There was a total of 15 positions in the final dataset. Ancestral sequence reconstruction was done with an expanded tree using the same methods. Evolutionary analyses were conducted in MEGA X (Kumar et al., Mol. Biol. Evol. 35, 1547-1549, 2018).

Cloning. The VEGAS adapted method was used to create different forms of AtARCSc plasmids (Mitchell et al., Nucleic Acids Res. 43, 6620-6630, 2015). A plasmid was used containing LisH H1 fused to AtIAA13 and expressing URA3 as a backbone in LisH H1 plasmid library construction. Plasmids were synthesized and confirmed with sequencing by Twist Bioscience (twistbioscience.com). All plasmids were transformed into reported haploid strain URA::[pRPS2-AtAFB2-ttClT1, LEU2, pADH1-ARF19-ttADH1, pP3(2×)-UbiVenus-ttCYC1]. A standard lithium acetate protocol (Gietz & Woods, Methods Enzymol. 350, 87-96, 2002) was used for transformations of digested plasmids. All cultures were grown at 30° C. with shaking at 220 rpm. For construction of plant vectors, the MoClo toolkit was used to design and clone plasmids containing the top 10 most repressive IAA13-H1s into vector plCH86966 by golden gate cloning (Weber et al., PLOS ONE 6, e16765, 2011). These were transformed into A. tumefaciens strain GV3101 via electroporation.

Library Design. Phylogenetic library contains sequences selected from the Pfam LisH alignment PF08513 using the representative protein database (RP15, 1,235 sequences, pfam.xfam.org/family/PF08513). Ancestrally reconstructed sequences contain synthetic sequences predicted at nodes 1-V using MEGAX node reconstruction software. HsTBL1 and HsDCAF1 LisH Helix 1 mutational libraries contain somatic mutations found in human cancer cells within these helixes and were identified using COSMIC datasets (Tate et al., Nucleic Acids Res. 47, D941-D947, 2019, cancer.sanger.ac.uk/cosmic). TPL site-saturation mutational libraries at residues TPLH1 R6 and F10 contain synthetic sequences probing the function of these sites in helix 1. The alpha helix control sequence (EAAAK)₃ (SEQ ID NO: 4) was created based on well-studied synthetic alpha helix linkers (Chen et al., Adv. Drug Deliv. Rev. 65, 1357-1369, 2013).

Flow Cytometry. Fluorescence measurements were taken using a Becton Dickinson (BD) special order cytometer with a 514-nm laser excitation fluorescence that is cut off at 525 nm prior to photomultiplier tube collection (BD, Franklin Lakes, NJ). Events were annotated, subset to singlet yeast using the FlowTime R package (github.com/wrightrc/flowTime). A total of 10,000-20,000 events above a 400,000 FSC-H threshold (to exclude debris) were collected for each sample and data exported as FCS 3.0 files for processing using the flowCore R software package and custom R scripts (Havens et al., Plant Physiol. 160, 135-142, 2012; Pierre-Jerome et al., Methods Mol. Biol. 1497, 271-281, 2017). Data from at least two independent replicates were combined and plotted in R (ggplot2.tidyverse.org/).

Yeast Methods. Standard yeast drop-out and yeast extract-peptone-dextrose plus adenine (YPAD) media were used, with care taken to use the same batch of synthetic dropout (SDO) media for related experiments. Haploid transformants were selected on appropriate prototrophy (SDO-Tryptophan, -Leucine). Yeast were grown at 30° C. on selection plates for two days, and in SDO liquid media with 250 rpm in a deep well 96-well plate format overnight for cytometry analysis (Pierre-Jerome et al., Methods Mol. Biol. 1497, 271-281, 2017). Liquid cultures were diluted 1:200 with fresh SDO the morning of cytometry analysis and measured after 5 hours of growth to a concentration of 200-500 events/μL.

Western Blot. Remaining yeast cultures from cytometry assays were diluted to OD₆₀₀=0.6 and incubated until cultures reached OD₆₀₀ 1. Cells were harvested by centrifugation. Cells were lysed by vortexing for 5 min in the presence of 200 μl of 0.5-mm diameter acid washed glass beads and 200 μl SUMEB buffer (1% SDS, 8 M urea, 10 mM MOPS pH 6.8, 10 mM EDTA, 0.01% bromophenol blue, 1 mM PMSF) per one OD unit of original culture. Lysates were then incubated at 65° C. for 10 min and cleared by centrifugation prior to electrophoresis and blotting. Antibodies: anti-HA-HRP (REF-12013819001, Clone 3F10, Roche/Millipore Sigma, St. Louis, MO), anti-PGK1 (ab113687, AbCam). Protein concentrations were quantified using ImageJ, with PGK1 protein measured in each strain to normalize protein concentrations across strains. To compare protein concentrations to AtTPL H1, these were then normalized to TPL concentration using this equation: ([X H1]/[PGK1])/[AtTPL H1]. AtTPL H1 normalized protein concentrations were plotted on a Log 2 scale.

Plant growth. For synthetic repression assays in tobacco, Agrobacterium-mediated transient transformation of N. benthamiana was performed as per (Yang et al., Plant J. 22, 543-551, 2000). 5 ml cultures of Agrobacterium strains were grown overnight at 30° C. shaking at 220 rpm, pelleted, and incubated in MMA media (10 mM MgCl₂, 10 mM MES pH 5.6, 100 μM acetosyringone) for 3 hours at room temperature with rotation. Strain density was normalized to an OD₆₀₀ of 1 for each strain in the final mixture of strains before injection into tobacco leaves. Leaves were removed, and eight different regions were excised using a hole punch, placed into a 96-well microtiter plate with 100 μl of water. Each leaf punch was scanned in a 4×4 grid for yellow and red fluorescence using a plate scanner (Tecan Spark, Tecan Trading AG, Switzerland). Fluorescence data was quantified and plotted in R (ggplots2).

### Example 2: Small Molecule Testing on the Yeast H1 Platform

This example describes use of the yeast H1 platform described herein to test BC2059/Tegavivint, an exemplar small molecule.

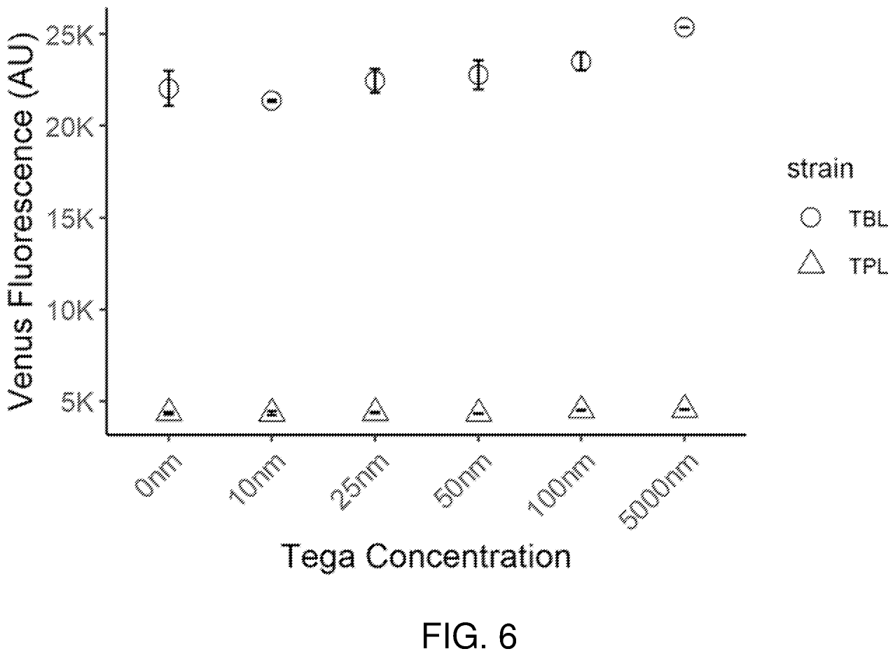

Yeast were grown overnight from a dilution of 1 cell per milliliter at 30° C. and 250 rpm. When cell density reached 200 cells per milliliter either control (DMSO) or small molecule BC2059 were added at the indicated concentration, and cultured for 4 hours before being measured for fluorescence by flow cytometry. Results are presented in FIG. 6. Each data point represents three independent time-course flow cytometry experiments of the helices indicated, all fused to IAA3. Every point represents the average fluorescence of at least 10,000 individually measured yeast cells (a.u.: arbitrary units). Error bars are standard error.

This experiment demonstrates that the ARC is amenable to addition of small molecule modifiers of activity, illustrated here using the small molecule BC2059 or Tegavivint, which is documented to bind to the H1 sequence of TBL1 and its homologs. The results show dose-dependent interference of TBL1's repressive activity on transcription, even in the absence of TBL1s binding partner Beta-catenin. This suggests that modulation of H1's transcriptional activity is detectable by small molecule perturbation. This is a method that can detect H1-specific interaction, as the TPL sequence is unaffected by the presence of the BC2059 molecule.

### Example 3: Cancer Variant Detection Using the Yeast H1 Platform

This example describes use of the yeast H1 platform described herein to test different TBL1 H1 variants, such as those found in certain cancers. It tests the responsiveness of different cancer variant mutations to the small molecule Tegavivint.

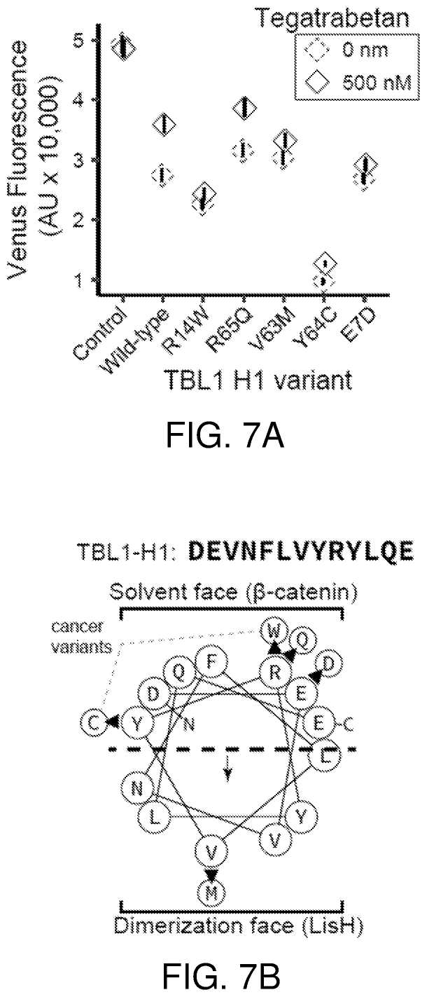

Yeast were grown overnight from a dilution of 1 cell per milliliter at 30° C. and 250 rpm. When cell density reached 200 cells per milliliter either control (DMSO) or small molecule BC2059 were added at the indicated concentration, and cultured for 4 hours before being measured for fluorescence by flow cytometry. Results are presented in FIGS. 7A-7B. Each data point represents three independent time-course flow cytometry experiments of the TBL1 or control helices indicated, all fused to IAA3. Every point represents the average fluorescence of at least 10,000 individually measured yeast cells (a.u.: arbitrary units). Error bars are standard error.

Yeast carrying either the wild type or mutant TBL1 H1 sequence were cultured in the presence or absence of 500 nM Tegatrabetan and fluorescence measured by flow cytometry. Certain cancer mutants were less sensitive to treatment. This suggests that they may lie in the binding site for Tegatrabetan, and that these variants could be used to screen for small molecules; such methods could be mutation specific. This approach is particularly relevant to personalized medicine for a given mutation in a patient.

Table 1 provides, from left to right: row number, H1 sequence, sequence identifier, protein name, and (if any) an alternate name.

TABLE 1
Characteristics of H1 Sequences

| # | Sequence | SEQ ID NO: | Protein | Alternate name |
|---|---|---|---|---|
| 1 | RELVFLILQFLDE | 8 | AtTPL | |
| 2 | RDVIKIMLQFCKE | 9 | AtSMU1 | |
| 3 | QEINRLILEYLVV | 10 | SmRANB9 | |
| 4 | SDVIRLIMQYLKE | 11 | HsSMU1 | |
| 5 | KELLLLIRNHLIS | 12 | HsDCAF1 | HsDCAF1 wt |
| 6 | KELLLLIHEHLQA | 13 | AtDCAF1 | |
| 7 | QQLYQLIYQHLIA | 14 | AmMAHJX1 | Q7PZX5_ANOGA |
| 8 | KELLQLIHDHLVS | 15 | LuDDB1 | |
| 9 | KNLLILILHYLTQ | 16 | MmKatnal | |
| 10 | NQVNYIIWRYLKE | 17 | SpHIF2 | |
| 11 | NDINLLIYHYLKE | 18 | Sc15387 | A0A1R2C3W4 |
| 12 | DEINFLIYKYLLE | 19 | DdQ54VL8 | Q54VL8 |
| 13 | EELNYLIWRYCQE | 20 | ScSIF2 | |
| 14 | DELNYLIYRYLLE | 21 | Gt158676 | L1IJL3 |
| 15 | EELNYLIYTYFLE | 22 | PtA0BHJ4 | |
| 16 | VELNFLVFRYLQE | 23 | AtHOS15 | |
| 17 | TELNYLVFRYLHE | 24 | GmK7KI15 | |
| 18 | QELNFLIFRYLHE | 25 | MpAXG93_328 | A0A176VRL0 |
| 19 | DVVNYLIYRYLQE | 26 | CrA0A2K3D4J8 | |
| 20 | DVINYLVMRYLQE | 27 | Ceg2227.t1 | A0A250WW80 |
| 21 | DEVNCLIYAYFQD | 28 | GfTBL1XR1 | |
| 22 | DEVNYLVYRYLIE | 29 | CIT1FMI0 | |
| 23 | DEVNYLVYRYLQE | 30 | TuTBL1XrR1 | |
| 24 | DEVNILVHRYLVE | 31 | NgTBL1XR1 | |
| 25 | DEVNLLVYRYLEE | 32 | GpA0A183C4K1 | A0A183C4K1 |
| 26 | EEVNFLVYRYLQE | 33 | EsD7FM70 | D7FM70 |
| 27 | DEVNFLVYRYLQE | 34 | HsTBL1X | |
| 28 | EEVNLLIYQYLIE | 35 | HdTBL1XR1 | A0A1W0X8L9 |
| 29 | KDLNKLIMNYLVI | 36 | CcPSD | A0A507F2M4 |
| 30 | SDVNSLILDYLVI | 37 | SpGID8 | |
| 31 | ADLNRLVMNYLVT | 38 | DsGID8 | DmeI |
| 32 | ADMNRLIMNYLVT | 39 | HsGID8 | GID8/TWA1 |
| 33 | EDMNKLIMDFFLT | 40 | MpGID8 | A0A2R6X9F0 |
| 34 | EDMNRLVMNFLVT | 41 | ZmGID8 | |
| 35 | EDMNTLVMNFLVT | 42 | AtGID8 | A8MQN5_ARATH |
| 36 | EDMNKLVMNFLVT | 43 | VvGID8 | W1P668 |
| 37 | EDMNRLVMNFLVV | 44 | AtGIDH | F4I7D4_ARATH |
| 38 | ADLNSIVMDYLIV | 45 | YIQ6C9J2 | |
| 39 | KDLNLVVLRYLLD | 46 | AcTBL1XR1 | A0A199VR62 |
| 40 | SDLNRIVLEYLNK | 47 | ScTAF5 | TAFII90 |
| 41 | EDLNRIVLSYLKK | 48 | KnTAF1 | |
| 42 | RLLSALICEYLDW | 49 | AtTON1B | |
| 43 | KLINQMIMEFLDW | 50 | DmCG5614 | |
| 44 | SLLNSYIYDYLIK | 51 | SpADN2 | |
| 45 | ELLNAYIYDYLLK | 52 | YlsomA | Q6C778 |
| 46 | QLLYAHIYNYLIK | 53 | ScMSS11 | |
| 47 | ESLDYYIYDYFVK | 54 | SpADN3 | |
| 48 | NALDFYIYNYMLK | 55 | PuLUGL | A0A176W7U0 |
| 49 | KMLDVYIYDYLVK | 56 | AtLUH | |
| 50 | KMLDVYIYDYFVK | 57 | ZmLUH | |
| 51 | KMLDVYIYDYLMK | 58 | MpLUH | A0A2R6X6P7 |
| 52 | KMLDVYIYDYLLK | 59 | OsLUH | |
| 53 | KMLDVYIHDYLVK | 60 | AtLUG | |
| 54 | KMLDVYIHDYLTK | 61 | SmLUH | D8RIG0 |
| 55 | EELNSALAEYLQR | 62 | BxLIS1 | |
| 56 | DELNRAIADYLRS | 63 | HsLIS1 | |
| 57 | QRVDRLIIDYLLR | 64 | SmMAEA | |
| 58 | ERIDSLIYGYLRR | 65 | CsQ20453 | |
| 59 | VRLNRLVADYMMA | 66 | SpFYV10 | |
| 60 | SELLGLVKEYLDF | 67 | MmARMC9 | |
| 61 | ELIQQLVLQFLQH | 68 | NcSSH4 | |
| 62 | TMIQKMVSSYLVH | 69 | HsRANBP9 | HsRANB9 |
| 63 | QVLNSLILDFLVK | 70 | CaFLO8 | |
| 64 | EFLNELISSFLLN | 71 | SpYC5C | |

Using the same row numbering as in Tables 1 & 3, Table 2 provides: row number, function, localization of the listed H1-containing proteins, a Uniprot-searchable name for each gene, and the species for each.

TABLE 2
Further Characteristics of H1 Sequences

| # | Function | Localization | Uniprot Searchable | Species |
|---|---|---|---|---|
| 1 | Repressor | Nuclear | TPL_ARATH | Arabidopsis thaliana |
| 2 | Other | Nuclear | SMU1_ARATH | Arabidopsis thaliana |
| 3 | Unknown | Unknown | G4VEU8_SCHMA | Schistosoma mansoni |
| 4 | Other | Nuclear | SMU1 | Homo sapiens (Human) |
| 5 | Other | Nuclear | Q9Y4B6 | Homo sapiens (Human) |
| 6 | Other | Nuclear | DCAF1_ARATH | Arabidopsis thaliana |
| 7 | Unknown | Unknown | AgaP_AGAP012134 | Anopheles merus |
| 8 | Unknown | Unknown | A0A1S3K6T0 | Lingula unguis |
| 9 | Other | Non- | KATL2_MOUSE | Mus musculus |
| | | nuclear | | |
| 10 | Repressor | Nuclear | HIF2_SCHPO | Schizosaccharomyces |
| | | | | pombe |
| 11 | Unknown | Unknown | SteCoe_15387 | Stentor coeruleus |
| 12 | Unknown | Unknown | DDB0206475_DICDI | Dictyostelium discoideum |
| 13 | Repressor | Nuclear | SIF2_YEAST | Saccharomyces cerevisiae |
| 14 | Unknown | Unknown | GUITHDRAFT_158676 | Guillardia theta |
| 15 | Unknown | Unknown | A0BHJ4 | Paramecium tetraurelia |
| 16 | Repressor | Nuclear | HOS15_ARATH | Arabidopsis thaliana |
| 17 | Unknown | Nuclear | K7KI15_GLYMA | Glycine max |
| 18 | Unknown | Unknown | AXG93_328s1070 | Marchantia polymorpha |
| | | | | subsp. ruderalis |
| 19 | Unknown | Unknown | A0A2K3D4J8_CHLRE | Chlamydomonas reinhardtii |
| 20 | Unknown | Unknown | CEUSTIGMA_g2227.t1 | Chlamydomonas eustigma |
| 21 | Unknown | Unknown | A0A1C7LUX2 | Grifola frondosa |
| 22 | Unknown | Unknown | T1FMI0_HELRO | Helobdella robusta |
| 23 | Unknown | Unknown | T1K4V1 | Tetranychus urticae |
| 24 | Unknown | Unknown | W7TZ27_9STRA | Nannochloropsis gaditana |
| 25 | Unknown | Unknown | GLOPA | Globodera pallida |
| 26 | Unknown | Unknown | ECTSI | Ectocarpus siliculosus |
| 27 | Activator/ | Nuclear | TBL1X_HUMAN | Homo sapiens (Human) |
| | Repressor | | | |
| 28 | Unknown | Unknown | RAMVA_A0A1D1V3Z7 | Hypsibius dujardini |
| 29 | Unknown | Unknown | BCR36DRAFT_350509 | Chytriomyces confervae |
| 30 | Unknown | Unknown | YDED_SCHPO | Schizosaccharomyces |
| | | | | pombe |
| 31 | Unknown | Unknown | B4IML9 | Drosophila sechellia |
| 32 | Other | Nuclear | GID8_HUMAN | Homo sapiens (Human) |
| 33 | Unknown | Unknown | MARPO_0028s0043 | Marchantia polymorpha |
| 34 | Unknown | Unknown | A0A317YIJ7_MAIZE | Zea mays |
| 35 | Other | Other | GID8_ARATH | Arabidopsis thaliana |
| 36 | Unknown | Unknown | W1P668 | Amborella trichopoda |
| 37 | Unknown | Unknown | O23690_ARATH | Arabidopsis thaliana |
| 38 | Unknown | Unknown | YALI0_D10791g | Yarrowia lipolytica |
| 39 | Unknown | Unknown | TBL1XR1_ANACO | Ananas comosus |
| 40 | Activator | Nuclear | TAF5_YEAST | Saccharomyces cerevisiae |
| 41 | Unknown | Unknown | A0A1Y1HYL3 | Klebsormidium nitens |
| 42 | Other | Other | TON1B_ARATH | Arabidopsis thaliana |
| 43 | Unknown | Unknown | Q9VF40 | Drosophila melanogaster |
| 44 | Activator | Nuclear | ADN2_SCHPO | Schizosaccharomyces |
| | | | | pombe |
| 45 | Unknown | Unknown | YALI0_E03102g | Yarrowia lipolytica |
| 46 | Activator | Nuclear | MSS11_YEAST | Saccharomyces cerevisiae |
| 47 | Activator | Unknown | ADN3_SCHPO | Schizosaccharomyces |
| | | | | pombe |
| 48 | Unknown | Unknown | AXG93_146s1420 | Pyrus ussuriensis |
| 49 | Activator/ | Nuclear | LUH_ARATH | Arabidopsis thaliana |
| | Repressor | | | |
| 50 | Unknown | Unknown | F2ELW0_HORVV | Zea mays |
| 51 | Unknown | Unknown | MARPO_0033s0155 | Marchantia polymorpha |
| 52 | Unknown | Unknown | Q0JBT9 | Oryza sativa subsp. |
| | | | | japonica |
| 53 | Activator/ | Nuclear | LUG_ARATH | Arabidopsis thaliana |
| | Repressor | | | |
| 54 | Unknown | Unknown | SELMO_94317 | Selaginella moellendorffii |
| 55 | Unknown | Unknown | A0A1I7STR7 | Bursaphelenchus xylophilus |
| 56 | Other | Non- | LIS1_HUMAN | Homo sapiens (Human) |
| | | nuclear | | |
| 57 | Unknown | Unknown | A0A0N5AP93 | Syphacia muris |
| 58 | Unknown | Unknown | Q20453_CAEEL | Caenorhabditis elegans |
| 59 | Unknown | Nuclear | FYV10_SCHPO | Schizosaccharomyces |
| | | | | pombe |
| 60 | Other | Other | ARMC9 MOUSE | Mus musculus |
| 61 | Unknown | Unknown | Q7S7G6 | Neurospora crassa |
| 62 | Other | Nuclear | RANB9_HUMAN | Homo sapiens (Human) |
| 63 | Activator | Nuclear | FLO8_CANAL | Candida albicans |
| 64 | Unknown | Unknown | YC5C_SCHPO | Schizosaccharomyces |
| | | | | pombe |

Using the same row numbering as in Tables 1 & 2, Table 3 provides: row number, other genes with an identical H1 sequence, and a representative citation for annotated localization and function for each gene (“NONE” indicates those with uncharacterized function or localization).

TABLE 3
Further Characteristics of H1 Sequences

| # | Other genes | Citation |
|---|---|---|
| 1 | TmTPL, VvTPR4, AoTPR2, CsTPL, | DOI: 10.1126/science.1151461 |
| | CcTPR1, PaTPL, CmTPR2, AcTPR2, | |
| | CnTPR3, TuTPR2, over 100 proteins | |
| 2 | GwSMU1, HhSMU1, PpSMU1, HgSMU1, | DOI: 10.1104/pp.109.141705 |
| | MdSMU1, BsSMU1X2, GaSMU1X1, | |
| | GvSMU1, over 100 proteins | |
| 3 | A0A183T2Q9, A0A068YDU5, H2KNK2, | NONE |
| | A0A183B251, G4VEU8, A0A183VRA7, | |
| | G4VEU8_SCHMA | |
| 4 | Q2TAY7 | DOI: 10.1016/j.yexcr.2005.02.017 |
| 5 | PtDCAF1, CjDCAF1, NaDACF1, | doi.org/10.1016/j.molcel.2012.09.004 |
| | SaDCAF1, PcDACF1, and over 100 | |
| | proteins | |
| 6 | A0A178UYS1, Q9M086, DCAF1_ARATH | DOI: 10.1105/tpc.108.058891 |
| 7 | AaMAHJX1, AcMAHJX1, AmMAHJ, | NONE |
| | AaMAHJX2, XP_320393.4, KFB51317.1 | |
| 8 | A0A1S3K7Q1, A0A1S3IUX2 | NONE |
| 9 | F7DX12, F1M5A4, Q9D3R6, | DOI: 10.1371/journal.pgen.1007078 |
| | KATL2_MOUSE | |
| 10 | none | DOI: 10.1016/s0960-9822(98)70304-5 |
| 11 | A0A1R2C3W4, A0A1R2CB06, | NONE |
| | A0A1R2BT11 | |
| 12 | DDB_G0280261, DICPUDRAFT_29050 | NONE |
| 13 | P38262, SIF2_YEAST | doi.org/10.1016/j.jmb.2005.06.025 |
| 14 | BoCAD5229314.1 | NONE |
| 15 | A0DLD6, A0BHJ4 | NONE |
| 16 | Q9FN19, A0A178UQT7, HOS15_ARATH, | DOI: 10.1104/pp.18.01156 |
| | F2DCR6, A0A287I129, A0A287I0S4, | |
| | A0A287I0S0 | |
| 17 | A0A199W458, K7KI15, D8T4U4, D8RCD5 | NONE |
| 18 | A0A176VRL0 | NONE |
| 19 | TsFbxw7, CHLRE_12g527500v5, | NONE |
| | CiHXX76_004694, 10 proteins | |
| 20 | A0A250WW80 | NONE |
| 21 | KAH6916628.1 | NONE |
| 22 | none | NONE |
| 23 | A0A2D4BVA7, C1FJI2, T1K4V1 | NONE |
| 24 | NSK_003728 | NONE |
| 25 | MeCAD2177457.1, MgKAF7638710.1, | NONE |
| | MeCAD2168972.1 | |
| 26 | A0A024G5P9, F0YLG7 | NONE |
| 27 | T0QLT6, D0NVN2, A7S5U0, A0A2B4S828, | DOI: 10.1210/jc.2016-2531 |
| | A0A1J1IKJ5, Q95RJ9, A0A1B0FD47, | |
| | I0YVP9, H9JDI6, B7Q2X9, A0A1S3J4R8, | |
| | A0A0L8H107, K1RDI6, V4A8I8, Q7Q371, | |
| | A0A087UPF7, A0A2A4J0P6, | |
| | A0A1S4EKS5, E9GI91, K7IPS0, | |
| | A0A158NMP9, T1HP17, E0VC07, | |
| | A0A2J7QTE6, A0A067R4E4, | |
| | A0A226E5C7, A0A1W4XAC3, D6WDR4, | |
| | A0A1D2N2Y2, T1JMU6, A0A2G8JKT3, | |
| | A0A2G8LR25, C3ZEP1, O60907, S9XQS6, | |
| | A2RUX0, A5WVU0, A0A1D5PXG9, | |
| | A5PF33, F6S036, A0A226NGR2, | |
| | Q9QXE7, G3V6G5, A0A1D5NXL1, | |
| | Q9BQ87, Q9BZK7, A0A091KAK7, | |
| | D3ZNF4, Q8BHJ5, M7B1G6, F6WUH3, | |
| | W4XYV6, F6YCJ1 | |
| 28 | RAMVA_A0A1D1V3Z7 | NONE |
| 29 | KAG4099912.1, OUM64527.1, | NONE |
| | ORY12276.1, | |
| 30 | none | NONE |
| 31 | Q8SZN4 | NONE |
| 32 | GID8_CHICK, A0A087TXU7, | DOI: 10.7554/eLife.35528 |
| | A0A1S3JEZ4, A0A226NJJ8, C3ZQT3, | |
| | B7PS79, Q9VWS1, E9QHU7, Q6PC55, | |
| | M7BF90, T1J9E3, Q9NWU2, Q9D7M1, | |
| | Q6YDN8, F7CLX0, Q5ZKQ7, V8NIM7, | |
| | F7DIR3, E7FGY2 | |
| 33 | AXG93_2931s1420 | NONE |
| 34 | DKX38_017052, PR202_ga26473, | NONE |
| | Sadunf11G0026900, ZmGIH8, | |
| | ONL95757.1, GFY91837.1, C3L33_15476, | |
| | GFZ03098.1, C2845_PM01G08500, | |
| | XP_003558414.1, 89 proteins | |
| 35 | BRARA_K00274, CAF2133834.1, | DOI: 10.1186/1471-2229-12-83 |
| | CAA7029683.1, Bca52824_086799, | |
| | HID58_043154, DY000_02041394, | |
| | BnaA03g60080D, XP_010418067.1, | |
| | EsGIDH, CsGID8, CrGID8X1, 49 proteins | |
| 36 | VvGIDH, KAG7019632.1, TEA_010235, | NONE |
| | E3N88_37174, FEM48_Zijuj01G0148200, | |
| | Ahy_B03g063295, F2P56_009768, | |
| | KAA0056956.1, KAE8716948.1, | |
| | E3N88_41952, CcGID8, CcGIDH, | |
| | CBI18750.3, 100 proteins | |
| 37 | CAE5957163.1, AXX17_AT1G11330, | NONE |
| | KAG7591256.1, AIGID81X, | |
| | KAG7653870.1, NP_001318979.1, | |
| | EFH66107.1, KAG7645900.1, | |
| | AAO63342.1, NP_001321853.1, | |
| | CAD5312385.1, 19 proteins | |
| 38 | Q6C9J2 | NONE |
| 39 | CAD1841307.1, AcHOS15 | NONE |
| 40 | P38129, A0A1E3PKV2, W1Q9X6, | DOI: 10.1016/s0092-8674(00)81220-9 |
| | A0A1D8PSS1, KOKTJ7, S6ENA4, | |
| | A0A167EJ71, C4R4L4 | |
| 41 | A0A1Y1HYL3 | NONE |
| 42 | CIPAW_02G177500, I3760_02G177300, | doi: 10.1105/tpc.107.056812 |
| | JrTON1A, 13760_02G177300, CiTON1A, | |
| | JmTON1A, Leryth_016134, | |
| | CAD5325854.1, CmTON1AX1, AITON1A, | |
| | 44 proteins | |
| 43 | AT18024p | NONE |
| 44 | NONE | DOI: 10.1128/EC.00078-09 |
| 45 | FOA43_001062, HII12_001016, | NONE |
| | BKA90DRAFT_133770, | |
| | B0I71DRAFT_126502, YALI2_E00070g, | |
| | YALI0E03102p, BRETT_003633, | |
| | CJU89_1027, 9 proteins | |
| 46 | CAD6641633.1, AJS95620.1, Samss11p, | DOI: 10.1046/j.1365- |
| | YJM1443, YJM1389, JM1399, YJM320, | 2958.2003.03247.x |
| | AJS90831.1, AJS85596.1, SpMss11, | |
| | AJS66828.1, AJS89087.1, AJS93898.1, | |
| | AJS84716.1, 54 proteins | |
| 47 | ADN3_SCHPO | DOI: 10.1371/journal.pgen.1003104 |
| 48 | MARPO_0023s0181 | NONE |
| 49 | A0A287T3L7, A0A287T3B8, A0A287T3E2, | DOI: 10.1105/tpc.19.00115, DOI: |
| | A0A287T3D2, A0A287T3C9, | 10.1186/1471-2229-14-54 |
| | A0A287T3M2, A0A287T3B7, A0A199V044, | |
| | Q10A93, K7K8T5, I1LA80, O48847, | |
| | A0A178VQH2, A0A2K3P919, LUH_ARATH | |
| 50 | A0A287L2N7, A0A287L2N6, F2ELW0, | NONE |
| | A0A287L2M9, A0A287L2Q9, 75 proteins | |
| 51 | COLO4_34959, F8388_006400, | NONE |
| | DVH24_017861, RZB52406.1, | |
| | RZB52407.1, PuLUGL, DVH24_029234, | |
| | POE58772.1, QsLUG, | |
| | C4D60_Mb07t20460, JHK85_023989, | |
| | CAG1855999.1, CsLUG1X, 100 proteins | |
| 52 | TuLUG, MQM01692.1, | NONE |
| | ZmLEGLC5167_041388, | |
| | CY35_06G035600, BDL97_06G036400, | |
| | C5167_015243, MQL71672.1, | |
| | OIW03074.1, XP_019457374.1, LaLUGL, | |
| | 100 proteins | |
| 53 | B9RPC5_RICCO, A0A2J6KP98, | DOI: 10.1073/pnas.230352397 |
| | A0A178UVW2, A0A251SRG4, | |
| | A0A251UB06, A0A0R0FPP1, | |
| | A0A0R0JDA7, A0A2K3MNX1, D8SAR0, | |
| | D8QW58, S8CL12, I1M9M2, S8DZ67, | |
| | Q9FUY2 | |
| 54 | SmLUGX1, X2, X3, X4 | NONE |
| 55 | , CAD5215296.1 | NONE |
| 56 | Q803D2, LIS1_BOVIN, Q7T394, Q9PTRZ, | doi.org/10.1091/mbc.e12-03-0210 |
| | A0A091JVU8, P43034, P63005, P63004, | |
| | F6PQP7, V8P437, Q6NZH4 | |
| 57 | VDD86692.1 | NONE |
| 58 | H2L2B2, EGT35366.1, NP_001256138.1, | NONE |
| | NP_001256137.1, NP_505524.2, | |
| | NP_872162.2, NP_001023935.1 | |
| 59 | NONE | DOI: 10.1038/nbt1222 |
| 60 | MXQ79359.1, MdARMC9, MbARMC9, | DOI: 10.1038/s41588-018-0054-7 |
| | KAF4014044.1, XP_037705437.1, | |
| | XP_037705438.1, XP_045054028.1, | |
| | XP_045054029.1, XP_022423649.1, | |
| | NaARMC9X1, PtARMC9X1, DIARMC9X2, | |
| | 100 proteins | |
| 61 | GQX73_g5821, CIB48_g11750, | NONE |
| | PODANS_2_950, KAH6631079.1, | |
| | PaRBP10, INS49_012262, SMAC_08383, | |
| | NEUTE1DRAFT_57335, NcRANB, 43 | |
| | proteins | |
| 62 | F1LVV3, P69566, M7BR83, Q96S59, | DOI: 10.1083/jcb.200801133 |
| | A0A091KE14, F7D8G8, A0A226NKE1 | |
| 63 | XP_002421174.1, W5Q_05038, | DOI: 10.1091/mbc.e05-06-0502 |
| | AAQ03244.1, MGS_04938, W5O_04937, | |
| | MG1_04938, MEU_04910, L150_04850, | |
| | MEY_04890, MGO_04880, MEW_04821, | |
| | MEM_04918, 29 proteins | |
| 64 | O94712 | NONE |

TABLE 4
Plasmids and corresponding H1 sequences for yeast; plasmids were
transformed into strain pNL4476.

| # | Yeast Plasmid | H1 Sequence | SEQ ID NO: |
|---|---|---|---|
| 3455 | c1-1_TPL_ARATH | RELVFLILQFLDE | 8 |
| 3454 | c9-2_DCAF1_ARATH | KELLLLIHEHLQA | 13 |
| 3453 | c17-3_LUH_ARATH | KMLDVYIYDYLVK | 56 |
| 3452 | c17-6_LUG_ARATH | KMLDVYIHDYLVK | 60 |
| 3451 | c14-4_HOS15_ARATH | VELNFLVFRYLQE | 23 |
| 3450 | c4-3_GID8_ARATH | EDMNTLVMNFLVT | 42 |
| 3448 | c4-8_O23690_ARATH | EDMNRLVMNFLVV | 44 |
| 3447 | c1-2_SMU1_ARATH_3 | RDVIKIMLQFCKE | 9 |
| 3446 | c20-1_TON1B_ARATH | RLLSALICEYLDW | 49 |
| 3445 | c15-9_TBL1X_HUMAN | DEVNFLVYRYLQE | 34 |
| 3444 | c18-2_LIS1_HUMAN | DELNRAIADYLRS | 63 |
| 3443 | c4-2_GID8_HUMAN | ADMNRLIMNYLVT | 39 |
| 3442 | c22-1_RANB9_HUMAN | TMIQKMVSSYLVH | 69 |
| 3441 | c10-1_KATL2_MOUSE | KNLLILILHYLTQ | 16 |
| 3440 | c1-3_SMU1_HUMAN | SDVIRLIMQYLKE | 11 |
| 3439 | c4-1_DmeI_DROME | ADLNRLVMNYLVT | 38 |
| 3438 | c19-1_MAEA_SYPMU | QRVDRLIIDYLLR | 64 |
| 3437 | c9- | QQLYQLIYQHLIA | 14 |
| | 3_AgaP_AGAP012134 | | |
| 3436 | c15-5_HELRO | DEVNYLVYRYLIE | 29 |
| 3435 | c18-1_Lis1_BURXY | EELNSALAEYLQR | 62 |
| 3434 | c9-1_DDB1_LINUN | KELLQLIHDHLVS | 15 |
| 3433 | c15-6_T1K4V1 | DEVNYLVYRYLQE | 30 |
| 3432 | c11-1_RAMVA | EEVNLLIYQYLIE | 35 |
| 3431 | c20-2_Q9VF40 | KLINQMIMEFLDW | 50 |
| 3430 | c15-7_GLOPA | DEVNLLVYRYLEE | 32 |
| 3429 | c3-1_BCR36DRAFT_ | KDLNKLIMNYLVI | 36 |
| | 350509 | | |
| 3428 | c23-1_YC5C_SCHPO | EFLNELISSFLLN | 71 |
| 3427 | c14-1_SIF2_YEAST | EELNYLIWRYCQE | 20 |
| 3426 | c5-1_FLO8_CANAL | QVLNSLILDFLVK | 70 |
| 3425 | c8-1_TAF5_YEAST | SDLNRIVLEYLNK | 47 |
| 3424 | c16-3_MSS11_YEAST | QLLYAHIYNYLIK | 53 |
| 3423 | c17-1_ADN3_SCHPO | ESLDYYIYDYFVK | 54 |
| 3422 | c12-1_HIF2_SCHPO | NQVNYIIWRYLKE | 17 |
| 3421 | c16-2_YALI0_E03102g | ELLNAYIYDYLLK | 52 |
| 3420 | c15-3_Tbl1xr1_RHVIN | DEVNCLIYAYFQD | 28 |
| 3419 | c16-1_ADN2_SCHPO | SLLNSYIYDYLIK | 51 |
| 3418 | c6-1_YALI0_D10791g | ADLNSIVMDYLIV | 45 |
| 3417 | c19-3_FYV10_SCHPO | VRLNRLVADYMMA | 66 |
| 3416 | c3-2_YDED_SCHPO | SDVNSLILDYLVI | 37 |
| 3415 | c21-1_SSH4_NUECR | ELIQQLVLQFLQH | 68 |
| 3414 | c4-6_A8HQD2_CHLRE | EDMNRLVMNFLVT | 41 |
| 3413 | c15- | DVVNYLIYRYLQE | 26 |
| | 1_A0A2K3D4J8_CHLRE | | |
| 3412 | c4-5_AMTR | EDMNKLVMNFLVT | 43 |
| 3411 | c4- | EDMNKLIMDFFLT | 40 |
| | 4_MARPO_0028s0043 | | |
| 3410 | c17-5_MARPO_ | KMLDVYIYDYLMK | 58 |
| | 0033s0155 | | |
| 3409 | c7-1_TBL1XR1_ANACO | KDLNLVVLRYLLD | 46 |
| 3408 | c17-7_SELMO_94317 | KMLDVYIHDYLTK | 61 |
| 3407 | c14-2_GUITH_158676 | DELNYLIYRYLLE | 21 |
| 3406 | c24-1_Q0JBT9 | KMLDVYIYDYLLK | 59 |
| 3405 | c17-4_F2ELWO_HORVV | KMLDVYIYDYFVK | 57 |
| 3404 | c15-4_tbl1xr1_NAGAD | DEVNILVHRYLVE | 31 |
| 3403 | c17- | QELNFLIFRYLHE | 25 |
| | 2.2_AXG93_328s1070 | | |
| 3402 | c15-2_CHEUS | DVINYLVMRYLQE | 27 |
| 3401 | c14-5_K7KI15_GLYMA | TELNYLVFRYLHE | 24 |
| 3400 | c17- | NALDFYIYNYMLK | 55 |
| | 2.1_AXG93_146s1420 | | |
| 3399 | c8-2_TAF1_KLENI | EDLNRIVLSYLKK | 48 |
| 3398 | c15-8_ECTSI | EEVNFLVYRYLQE | 33 |
| 3397 | c2-1_RANB9_SCHMA | QEINRLILEYLVV | 10 |
| 3396 | c19-2_CELE_CAEEL | ERIDSLIYGYLRR | 65 |
| 3395 | c14-3_A0BHJ4 | EELNYLIYTYFLE | 22 |
| 3394 | c13- | DEINFLIYKYLLE | 19 |
| | 1_DDB0206475_DICDI | | |
| 3393 | c12-2_SteCoe_15387 | NDINLLIYHYLKE | 18 |
| 3392 | TBL1XR1_E7D | DDVNFLVYRYLQE | 78 |
| 3391 | TBL1X_F61V | DEVNVLVYRYLQE | 79 |
| 3390 | TBL1X_V63M | DEVNFLMYRYLQE | 80 |
| 3389 | TBL1X_Y64C | DEVNFLVCRYLQE | 81 |
| 3388 | TBL1X_R65Q | DEVNFLVYQYLQE | 82 |
| 3387 | TBL1X_R14W | DEVNFLVYWYLQE | 83 |
| 3386 | HsDCAF1_wt | KELLLLIRNHLIS | 12 |
| 3385 | HsDCAF1_L851F | KELLFLIRNHLIS | 84 |
| 3384 | HsDCAF1_I853M | KELLLLMRNHLIS | 85 |
| 3383 | HsDCAF1_R854Q | KELLLLIQNHLIS | 86 |
| 3382 | HsDCAF1_H856Y | KELLLLIRNYLIS | 87 |
| 3381 | TPLH1_R6 | RELVFLILQFLDE | 8 |
| 3380 | TPLH1_R6H | HELVFLILQFLDE | 88 |
| 3379 | TPLH1_R6K | KELVFLILQFLDE | 89 |
| 3378 | TPLH1_R6D | DELVFLILQFLDE | 90 |
| 3377 | TPLH1_R6E | EELVFLILQFLDE | 91 |
| 3376 | TPLH1_R6S | SELVFLILQFLDE | 92 |
| 3375 | TPLH1_R6T | TELVFLILQFLDE | 93 |
| 3374 | TPLH1_R6N | NELVFLILQFLDE | 94 |
| 3373 | TPLH1_R6Q | QELVFLILQFLDE | 95 |
| 3372 | TPLH1_R6C | CELVFLILQFLDE | 96 |
| 3371 | TPLH1_R6G | GELVFLILQFLDE | 97 |
| 3370 | TPLH1_R6P | PELVFLILQFLDE | 98 |
| 3369 | TPLH1_R6A | AELVFLILQFLDE | 99 |
| 3368 | TPLH1_R61 | IELVFLILQFLDE | 100 |
| 3367 | TPLH1_R6L | LELVFLILQFLDE | 101 |
| 3366 | TPLH1_R6M | MELVFLILQFLDE | 102 |
| 3365 | TPLH1_R6F | FELVFLILQFLDE | 103 |
| 3364 | TPLH_R6W | WELVFLILQFLDE | 104 |
| 3363 | TPLH1_R6Y | YELVFLILQFLDE | 105 |
| 3362 | TPLH1_R6V | VELVFLILQFLDE | 106 |
| 3361 | Repressor | KEIIRLILQYLHE | 107 |
| 3360 | Transc. Regulator | EELNRLIMNYLMH | 108 |
| 3359 | Cytoskeletal | EELNRLIADYMQH | 109 |
| 3358 | Activator | NMLNVLIYDYLIH | 110 |
| 3357 | Basal | KLINQMIMEYLEW | 111 |
| 3356 | ARMC9_Mm | SELLGLVKEYLDF | 67 |
| 3355 | alphahelix | EAAAKEAAAKEAAAK | 4 |
| 3353 | TPLH1_F10H | RELVHLILQFLDE | 113 |
| 3352 | TPLH1_F10D | RELVDLILQFLDE | 114 |
| 3351 | TPLH1_F10E | RELVELILQFLDE | 115 |
| 3350 | TPLH1_F10T | RELVTLILQFLDE | 116 |
| 3349 | TPLH1_F10N | RELVNLILQFLDE | 117 |
| 3348 | TPLH1_F10Q | RELVQLILQFLDE | 118 |
| 3347 | TPLH1_F10C | RELVCLILQFLDE | 119 |
| 3346 | TPLH1_F10G | RELVGLILQFLDE | 120 |
| 3345 | TPLH1_F10A | RELVALILQFLDE | 121 |
| 3344 | TPLH1_F101 | RELVILILQFLDE | 122 |
| 3343 | TPLH1_F10L | RELVLLILQFLDE | 123 |
| 3341 | TPLH1_F10W | RELVWLILQFLDE | 124 |
| 3340 | TPLH1_F10V | RELVVLILQFLDE | 125 |
| 3339 | TPL_2xH1 | RELVFLILQFLDEGGGGSGGGGSRELV | 126 |
| | | FLILQFLDE | |
| 3338 | TPL_3xH1 | RELVFLILQFLDEGGGGSGGGGSRELV | 127 |
| | | FLILQFLDEGGGGSGGGGSRELVFLIL | |
| | | QFLDE | |
| 3337 | TPL_F10A | RELVALILQFLDE | 121 |
| 3336 | TPL_F10R | RELVRLILQFLDE | 128 |
| 3335 | TPLH1_REDE > AAAA | AALVFLILQFLAA | 129 |
| 3334 | TPL_4xH1 | RELVFLILQFLDEGGGGSGGGGSRELV | 130 |
| | | FLILQFLDEGGGGSGGGGSRELVFLIL | |
| | | QFLDEGGGGSGGGGSRELVFLILQFLD | |
| | | E | |

TABLE 5
Plasmids and corresponding H1 sequences for Agrobacterium; plasmids were
transformed into strain pICH86966.

| # | Agrobacterium Plasmid | H1 Sequence | SEQ ID NO: |
|---|---|---|---|
| 3548 | p35SLong_5U:[H1-AtTPL-F10A]-HA-IAA3- | RELVALILQFLDE | 121 |
| | 3U + Ter-AtuNos | | |
| 3547 | p35SLong_5U:[H1-AtTPL-F10G]-HA-IAA3- | RELVGLILQFLDE | 120 |
| | 3U + Ter-AtuNos | | |
| 3546 | p35SLong_5U:[H1-AtTPL-F10H]-HA-IAA3- | RELVHLILQFLDE | 113 |
| | 3U + Ter-AtuNos | | |
| 3545 | p35SLong_5U:[H1-AtTPL-R6A]-HA-IAA3- | AELVFLILQFLDE | 99 |
| | 3U + Ter-AtuNos | | |
| 3544 | p35SLong_5U:[H1-AtTPL-R6Y]-HA-IAA3- | YELVFLILQFLDE | 105 |
| | 3U + Ter-AtuNos | | |
| 3543 | p35SLong_5U:[H1-AtTPL-R6L]-HA-IAA3- | LELVFLILQFLDE | 101 |
| | 3U + Ter-AtuNos | | |
| 3542 | p35SLong_5U:[H1-alphaH]-HA-IAA3- | EAAAKEAAAKEAAAK | 4 |
| | 3U + Ter-AtuNos | | |
| 3541 | p35SLong_5U:[H1-AtHOS15]-HA-IAA3- | VELNFLVFRYLQE | 23 |
| | 3U + Ter-AtuNos | | |
| 3540 | p35SLong_5U:[H1-RANB9-schma]-HA- | QEINRLILEYLVV | 10 |
| | IAA3-3U + Ter-AtuNos | | |
| 3539 | p35SLong_5U:[H1-DmDMEI]-HA-IAA3- | ADLNRLVMNYLVT | 38 |
| | 3U + Ter-AtuNos | | |
| 3538 | p35SLong_5U:[H1-BCR36DRAFT]-HA- | KDLNKLIMNYLVI | 36 |
| | IAA3-3U + Ter-AtuNos | | |
| 3537 | p35SLong_5U:[H1-A0A2K3D4J8]-HA- | DVVNYLIYRYLQE | 26 |
| | IAA3-3U + Ter-AtuNos | | |
| 3536 | p35SLong_5U:[H1-TAF1]-HA-IAA3- | EDLNRIVLSYLKK | 48 |
| | 3U + Ter-AtuNos | | |
| 3535 | p35SLong_5U:[H1-FLO8]-HA-IAA3- | QVLNSLILDFLVK | 70 |
| | 3U + Ter-AtuNos | | |
| 3534 | p35SLong_5U:[H1-AtTPL]-HA-IAA3- | RELVFLILQFLDE | 8 |
| | 3U + Ter-AtuNos | | |
| 3533 | p35SLong_5U:[H1-DDB0206475]-HA- | DEINFLIYKYLLE | 19 |
| | IAA3-3U + Ter-AtuNos | | |
| 3532 | p35SLong_5U:[H1-AtTON1B]-HA-IAA3- | RLLSALICEYLDW | 49 |
| | 3U + Ter-AtuNos | | |
| 3531 | p35SLong_5U:[H1-ADN2]-HA-IAA3- | SLLNSYIYDYLIK | 51 |
| | 3U + Ter-AtuNos | | |
| 3530 | p35SLong_5U:[H1-HsRANBP9]-HA-IAA3- | TMIQKMVSSYLVH | 69 |
| | 3U + Ter-AtuNos | | |
| 3529 | p35SLong_5U:[H1-HsSMU1]-HA-IAA3- | SDVIRLIMQYLKE | 11 |
| | 3U + Ter-AtuNos | | |
| 3528 | p35SLong_5U:[H1-SSH4]-HA-IAA3- | ELIQQLVLQFLQH | 68 |
| | 3U + Ter-AtuNos | | |
| 3527 | p35SLong_5U:[H1-DDB1]-HA-IAA3- | KELLQLIHDHLVS | 15 |
| | 3U + Ter-AtuNos | | |
| 3526 | p35SLong_5U:[H1-AtDCAF1]-HA-IAA3- | KELLLLIHEHLQA | 13 |
| | 3U + Ter-AtuNos | | |
| 3525 | p35SLong_5U:[H1-YC5C]-HA-IAA3- | EFLNELISSFLLN | 71 |
| | 3U + Ter-AtuNos | | |

### (XIII) CLOSING PARAGRAPHS

Unless otherwise indicated, the practice of the present disclosure can employ conventional techniques of immunology, molecular biology, microbiology, cell biology and recombinant DNA. These methods are described in the following publications. See, e.g., Sambrook et al., Molecular Cloning: A Laboratory Manual, 2nd Edition (1989); Ausubel et al., eds., Current Protocols in Molecular Biology (1987); the series Methods In Enzymology (Academic Press, Inc.); MacPherson et al., PCR: A Practical Approach, IRL Press at Oxford University Press (1991); MacPherson et al., eds. PCR 2: Practical Approach (1995); Harlow and Lane, eds. Antibodies, A Laboratory Manual (1988); and Freshney, ed., Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Use of the word(s), “exemplary” or “embodiment” or “desirably” in this document does not limit the definition or language with which the word(s) is used, and is intended to further illustrate in a non-limiting fashion meaning through use of an example or particular embodiments within the scope of the definition.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, public sequence database entries, and other written text throughout this specification (generally, “referenced materials”). Each of the referenced materials is individually incorporated herein by reference in its entirety for the referenced teaching(s).

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. A genetically modified eukaryotic cell comprising, on one or more expression constructs or integrated into the genome of the cell:
a sequence encoding an auxin receptor;
a sequence encoding an auxin response factor;
a sequence encoding a reporter; and
a sequence encoding a Lis1 Homology (LisH) domain fused to an auxin-responsive protein.

2. The genetically modified cell of claim 1, wherein at least one of the encoding sequences is an element of an expression construct, and optionally the expression construct is in the form of a plasmid.

3. The genetically modified cell of claim 1, wherein one or more of:
the auxin receptor has one or more of the following characteristics:
comprises an F-box domain and a leucine-rich repeat (LRR) domain;
binds auxin (indole-3-acetic acid);
is auxin-signaling F-box 2 (AFB2);
comprises a sequence having 50% sequence identity to the sequence set forth in SEQ ID NO: 1; and/or
the Lis1 Homology domain comprises:
a cancer variant,
a developmental variant, or
a Lis1 Homology domain from TOPLESS (TPL), TOPLESS-RELATED (TPR1, TPR2, TPR3, or TPR4), LEUNIG (LUG), LEUNIG homolog (LH), High Expression of Osmotically responsive genes 15 (HOS15), silencing mediator of retinoic acid and thyroid hormone receptor (SMRT), nuclear receptor corepressor (NCoR), Tup1, Groucho (Gro), or transducing-like enhancer (TLE); and/or
the auxin-responsive protein has one or more of the following characteristics:
comprises a Phox/Bem1p (PB1) domain and binds the auxin response factor;
comprises a sequence having 40% sequence identity to the sequence as set forth in SEQ ID NO: 3; or
is indoleacetic acid-induced protein 3 (IAA3).

4. The genetically modified cell of claim 1, wherein the auxin response factor has one or more of the following characteristics:
comprises a DNA-binding domain (DBD) and a Phox/Bem1p (PB1) domain;
binds the auxin-responsive protein and an auxin response element;
is auxin response factor 19 (ARF19); or
comprises a sequence having 50% identity to the sequence set forth in SEQ ID NO: 2.

5. The genetically modified cell of claim 4, wherein the auxin response element (when present) comprises a sequence upstream of the reporter comprising:
a TGTCxx sequence motif;
a TGTCxx sequence motif comprising TGTCTC; or
a TGTCxx sequence motif comprising TGTCGG.

6-11. (canceled)

12. The genetically modified cell of claim 2, wherein one or more of:
a first expression construct further comprises or encodes a selection marker;
a second expression construct further comprises or encodes a selection marker;
both the first and the second expression construct further comprises or encodes a selection marker;
a first expression construct and a second expression construct are on different plasmids;
a first and second expression constructs are on a single plasmid; or
at least one of the first and second expression constructs is integrated into the genome of the cell.

13-18. (canceled)

19. The genetically modified cell of claim 1, within a library, wherein the library comprises genetically modified cells transformed with a library of expression constructs, wherein each expression construct each comprises a LisH domain fused to an auxin-responsive protein.

20. The genetically modified cell of claim 1, wherein the LisH Domain comprises:
the sequence of any one of SEQ ID NOs: 8-111 or 113-130; or
an alpha-helix comprising an amino acid sequence XX[I/V/L]XX[Y/I/V/L][I/V/L]XXX[L]XX, wherein “X” can be any amino acid.

21. (canceled)

22. The genetically modified eukaryotic cell of claim 1, comprising:
(a) a first expression construct encoding the auxin receptor, the auxin response factor, and the reporter; and a second expression construct encoding the Lis1 Homology (LisH) domain fused to the auxin-responsive protein;
(b) a first expression construct encoding at least one of the auxin receptor, the auxin response factor, and/or the reporter; and a second expression construct encoding the Lis1 Homology (LisH) domain fused to the auxin-responsive protein;
(c) a first expression construct encoding at least one of the auxin receptor, the auxin response factor, and/or the reporter; and the sequence encoding the Lis1 Homology (LisH) domain fused to the auxin-responsive protein is integrated into the genome of the cell; or
(d) at least one of the sequence encoding the auxin receptor, the auxin response factor, and/or the reporter integrated into the genome of the cell; and an expression construct encoding the Lis1 Homology (LisH) domain fused to the auxin-responsive protein.

23. A method of determining repression activity comprising:
identifying or selecting a Lis1 Homology domain (LisH) sequence of interest;
synthesizing a plasmid wherein the plasmid comprises the LisH sequences of interest fused to an auxin-responsive protein;
transforming a eukaryotic cell with the plasmid to create a genetically modified cell; and
determining repression activity within the cell.

24. The method of claim 23, wherein one or more of:
the LisH sequence of interest comprises:
a cancer variant;
a developmental mutation variant; or
the Lis1 Homology domain of TOPLESS (TPL), TOPLESS-RELATED (TPR1, TPR2, TPR3, or TPR4), LEUNIG (LUG), LEUNIG homolog (LH), High Expression of Osmotically responsive genes 15 (HOS15), silencing mediator of retinoic acid and thyroid hormone receptor (SMRT), nuclear receptor corepressor (NCoR), Tup1, Groucho (Gro), or transducing-like enhancer (TLE); and/or
the auxin-responsive protein has one or more of the following characteristics:
comprises a Phox/Bem1p (PB1) domain and binds auxin response factor;
comprises a sequence having 40% sequence identity to the sequence set forth in SEQ ID NO: 3; or
is indoleacetic acid-induced protein 3 (IAA3); and/or
synthesizing a plasmid comprises a versatile genetic assembly system.

25-26. (canceled)

27. The method of claim 23, wherein the cell expresses an auxin receptor, an auxin response factor, and a reporter.

28. The method of claim 27, wherein:
the auxin receptor has one or more of the following characteristics:
comprises an F-box domain and a leucine-rich repeat (LRR) domain:
the auxin receptor binds auxin (indole-3-acetic acid);
is auxin-signaling F-box 2 (AFB2); or
comprises a sequence having 50% sequence identity to the sequence set forth in SEQ ID NO: 1; and/or
the auxin response factor has one or more of the following characteristics:
comprises a DNA-binding domain (DBD) and a Phox/Bem1p (PB1) domain;
binds the auxin-responsive protein and an auxin response element;
is auxin response factor 19 (ARF19); or
comprises a sequence having 50% identity to the sequence set forth in SEQ ID NO: 2.

29. (canceled)

30. The method of claim 28, wherein the auxin response element (when present) comprises a sequence upstream of the reporter comprising:
a TGTCxx sequence motif;
a TGTCxx sequence motif comprising TGTCTC; or
a TGTCxx sequence motif comprising TGTCGG.

31-33. (canceled)

34. The method of claim 23, wherein the plasmid further comprises: an auxin receptor, an auxin response factor, and a reporter, such that the genetically modified cell expresses the auxin receptor, the auxin response factor, and the reporter.

35. The method of claim 34, wherein one or more of:
the auxin receptor has one or more of the following characteristics:
comprises an F-box domain and a leucine-rich repeat (LRR) domain;
binds auxin (indole-3-acetic acid);
is auxin-signaling F-box 2 (AFB2); or
comprises a sequence having 50% sequence identity to the sequence set forth in SEQ ID NO: 1; and/or
the auxin response factor has one or more of the following characteristics:
comprises a DNA-binding domain (DBD) and a Phox/Bem1p (PB1) domain;
binds the auxin-responsive protein and an auxin response element;
is auxin response factor 19 (ARF19); or
comprises a sequence having 50% identity to the sequence set forth in SEQ ID NO: 2.

36. (canceled)

37. The method of claim 35, wherein the auxin response element (when present) comprises a sequence upstream of the reporter comprising:
a TGTCxx sequence motif;
a TGTCxx sequence motif comprising TGTCTC; or
a TGTCxx sequence motif comprising TGTCGG.

38-43. (canceled)

44. The method of claim 23, further comprising screening a bioactive molecule, wherein the screening comprises contacting the transformed cell with the bioactive molecule and determining repression activity.

45. The method of claim 44, wherein;
the bioactive molecule comprises one or more of:
a small molecule;
a peptide or protein;
a natural product;
a synthetic bioactive compound;
an anti-cancer drug; or
the anti-cancer drug BC 2059 (Tegavivint); and/or
determining repression activity comprises performing one or more of: a transcription-based assay; flow cytometry; a Western blot assay, microscopy, a fluorescence assay, or a luminescence assay.

46-51. (canceled)

52. The method of claim 23, wherein one or more of:
the cell has been transiently or stably transformed with the plasmid to produce the genetically modified cell;
the yeast cell is from a haploid strain or diploid strain;
the LisH sequence of interest comprises a library of LisH variants; and/or
the plasmid is a plasmid library of the library of LisH variants; and a plurality of cells are transformed with the plasmid library;
the LisH Domain comprises the sequence of any one of SEQ ID NOs: 8-111 or 113-130; and/or
the LisH Domain comprises an alpha-helix comprising an amino acid sequence XX[I/V/L]XX[Y/I/V/L][I/V/L]XXX[L]XX, wherein “X” can be any amino acid.

53-61. (canceled)
